(19) United States
(12) Patent Application Publication
(10) Pub. No.: US 2023/0378459 A1
(43) Pub. Date: Nov. 23, 2023

(54) SECONDARY BATTERY, ELECTRONIC DEVICE, AND VEHICLE

(71) Applicant: SEMICONDUCTOR ENERGY LABORATORY CO., LTD., Atsugi-shi, Kanagawa-ken (JP)

(72) Inventors: Kazutaka KURIKI, Ebina, Kanagawa (JP); Yumiko YONEDA, Isehara, Kanagawa (JP); Shiori SAGA, Atsugi, Kanagawa (JP); Yoshiharu ASADA, Odawara, Kanagawa (JP); Shunpei YAMAZAKI, Setagaya, Tokyo (JP)

(21) Appl. No.: 18/248,103

(22) PCT Filed: Oct. 13, 2021
(86) PCT No.: PCT/IB2021/059381

§ 371 Date: Apr. 6, 2023

(30) Foreign Application Priority Data
Oct. 26, 2020 (JP) 2020-179133

(51) Int. Cl. H01M 4/525; H01M 10/44; H01M 4/583; H01M 10/0569; H01M 4/133; C07D 233/58

(52) U.S. Cl. CPC H01M 4/525; H01M 10/44; H01M 4/583; H01M 10/0569; H01M 4/133; C07D 233/58; H01M 2004/028

(57) ABSTRACT

A lithium-ion secondary battery having high capacity and excellent charge and discharge cycle performance is provided. A secondary battery having high capacity is provided. A secondary battery with excellent charge and discharge characteristics is provided. A secondary battery in which a reduction in capacity is suppressed even when a state being charged with a high voltage is held for a long time is provided. In the secondary battery, after constant current charging is performed in an environment at 60° C. with a current value of 0.5 C until a voltage reaches 4.5 V, a charging process of performing constant voltage charging until a current value reaches 0.2 C and a discharging process of performing constant current discharging with a current value of 0.5 C until a voltage reaches 3 V are alternately repeated 150 or more times, and then discharging is performed, lithium cobalt oxide that is a surface portion of the positive electrode active material particle has an O3 structure, and an electrolyte includes an imidazolium cation.

### TECHNICAL FIELD

One embodiment of the present invention relates to an object, a method, or a manufacturing method. One embodiment of the present invention relates to a process, a machine, manufacture, or a composition of matter. One embodiment of the present invention relates to a semiconductor device, a display device, a light-emitting device, a power storage device, a lighting device, an electronic device, or a manufacturing method thereof. In particular, one embodiment of the present invention relates to a positive electrode active material that can be used for a secondary battery, a secondary battery, an electronic device including a secondary battery, and a vehicle including a secondary battery.

Another embodiment of the present invention relates to a power storage system including a secondary battery and a battery control circuit. Another embodiment of the present invention relates to an electronic device and a vehicle each including a power storage system.

Note that in this specification, a power storage device refers to every element and device having a function of storing power. Examples of the power storage device include a storage battery (also referred to as a secondary battery) such as a lithium-ion secondary battery, a lithium-ion capacitor, and an electric double layer capacitor.

In addition, electronic devices in this specification mean all devices including power storage devices, and electro-optical devices including power storage devices, information terminal devices including power storage devices, and the like are all electronic devices.

### BACKGROUND ART

In recent years, a variety of power storage devices such as lithium-ion secondary batteries, lithium-ion capacitors, and air batteries have been actively developed. In particular, demand for lithium-ion secondary batteries with high output and high energy density has rapidly grown with the development of the semiconductor industry, for portable information terminals such as mobile phones, smartphones, tablets, and laptop computers, portable music players, digital cameras, medical equipment, and next-generation clean energy vehicles (e.g., hybrid vehicles (HVs), electric vehicles (EVs), and plug-in hybrid vehicles (PHVs)), for example. The lithium-ion secondary batteries are essential as rechargeable energy supply sources for today's information society.

The performances required for lithium-ion secondary batteries are much higher energy density, improved cycle performance, safety under a variety of operation environments, and improved long-term reliability, for example.

In view of the above, improvement of positive electrode active materials has been studied to improve the cycle performance and increase the capacity of lithium-ion secondary batteries (Patent Document 1 and Patent Document 2). In addition, crystal structures of positive electrode active materials have been studied (Non-Patent Document 1 to Non-Patent Document 3).

Non-Patent Document 4 discloses the physical properties of metal fluorides.

X-ray diffraction (XRD) is one of methods used for analysis of a crystal structure of a positive electrode active material. With the use of the ICSD (Inorganic Crystal Structure Database) described in Non-Patent Document 5, XRD data can be analyzed.

### REFERENCES

### Patent Documents

[Patent Document 1] Japanese Published Patent Application No. 2002-216760
[Patent Document 2] Japanese Published Patent Application No. 2006-261132

### Non-Patent Documents

[Non-Patent Document 1] Toyoki Okumura et al., “Correlation of lithium ion distribution and X-ray absorption near-edge structure in O3- and O2-lithium cobalt oxides from first-principle calculation”, Journal of Materials Chemistry, 2012, 22, pp. 17340-17348.
[Non-Patent Document 2] Motohashi, T. et al., “Electronic phase diagram of the layered cobalt oxide system LiₓCoO₂ (0.0≤x≤1.0)”, Physical Review B, 80 (16); 165114.
[Non-Patent Document 3] Zhaohui Chen et al., “Staging Phase Transitions in LiₓCoO₂”, Journal of The Electrochemical Society, 2002, 149 (12) A1604-A1609.
[Non-Patent Document 4] W. E. Counts et al., “Fluoride Model Systems: II, The Binary Systems CaF₂—BeF₂, MgF₂—BeF₂, and LiF—MgF₂”, Journal of the American Ceramic Society (1953), 36 [1], 12-17. FIG. 01471.
[Non-Patent Document 5] Belsky, A. et al., “New developments in the Inorganic Crystal Structure Database (ICSD): accessibility in support of materials research and design”, Acta Cryst. (2002), B58, 364-369.

### SUMMARY OF THE INVENTION

### Problems to be Solved by the Invention

An object of one embodiment of the present invention is to provide a lithium-ion secondary battery having high capacity and excellent charge and discharge cycle performance, and a manufacturing method thereof. Another object of one embodiment of the present invention is to provide a secondary battery that can be rapidly charged, and a manufacturing method thereof. Another object of one embodiment of the present invention is to provide a high-capacity secondary battery, and a manufacturing method thereof. Another object of one embodiment of the present invention is to provide a secondary battery having excellent charge and discharge characteristics, and a manufacturing method thereof. Another object of one embodiment of the present invention is to provide a secondary battery in which a reduction in capacity is suppressed even when a state being charged with a high voltage is held for a long time, and a manufacturing method thereof. Another object of one embodiment of the present invention is to provide a highly safe or reliable secondary battery, and a manufacturing method thereof. Another object of one embodiment of the present invention is to provide a secondary battery in which a reduction in capacity is suppressed even at high temperatures, and a manufacturing method thereof. Another object of one embodiment of the present invention is to provide a long-life secondary battery, and a manufacturing method thereof.

An object of one embodiment of the present invention is to provide a safe, long-life, and extremely excellent secondary battery that can be rapidly charged, can be used at high temperatures, and can have a high energy density due to increased charge voltage.

An object of one embodiment of the present invention is to provide a positive electrode active material that has high capacity and excellent charge and discharge cycle performance for a lithium-ion secondary battery, and a manufacturing method thereof. Another object of one embodiment of the present invention is to provide a method for manufacturing a positive electrode active material with high productivity. Another object of one embodiment of the present invention is to provide a positive electrode active material that suppresses a reduction in capacity in charge and discharge cycles when used for a lithium-ion secondary battery. Another object of one embodiment of the present invention is to provide a positive electrode active material in which elution of a transition metal such as cobalt is suppressed even when a state being charged with a high voltage is held for a long time.

Another object of one embodiment of the present invention is to provide a novel material, novel active material particles, a novel power storage device, or a manufacturing method thereof.

Note that the description of these objects does not preclude the existence of other objects. One embodiment of the present invention does not have to achieve all these objects. Note that other objects can be taken from the description of the specification, the drawings, and the claims.

### Means for Solving the Problems

One embodiment of the present invention is a secondary battery including a positive electrode active material particle and an electrolyte. In the secondary battery, after constant current charging is performed in an environment at 60° C. with a current value of 0.5 C (note that 1 C=210 mA/g is satisfied) until a voltage reaches 4.5 V, a charging process of performing constant voltage charging until a current value reaches 0.2 C and a discharging process of performing constant current discharging with a current value of 0.5 C until a voltage reaches 3 V are alternately repeated 150 or more times, and then discharging is performed, lithium cobalt oxide that is a surface portion of the positive electrode active material particle has an O3 structure, and the electrolyte includes an imidazolium cation. In the above structure, it is preferable that a negative electrode be included and the negative electrode include graphite. In the above structure, it is preferable that the negative electrode include a current collector and a negative electrode active material layer over the current collector and a proportion of the graphite to total weight of the negative electrode active material layer be 50 weight % or more, 70 weight % or more, or 80 weight % or more.

Another embodiment of the present invention is a secondary battery including a positive electrode active material particle and an electrolyte. In the secondary battery, after constant current charging is performed in an environment at 20° C. or higher and 60° C. or lower, e.g., 25° C., 45° C., or 60° C. with a current value of 0.5 C (note that 1 C=210 mA/g is satisfied) until a voltage reaches 4.55 V or higher and 4.7 V or lower, e.g., 4.6 V with reference to lithium metal, a charging process of performing constant voltage charging until a current value reaches 0.2 C and a discharging process of performing constant current discharging with a current value of 0.5 C until a voltage reaches 2.5 V or higher and 3.2 V or lower, e.g., 3 V with reference to lithium metal are alternately repeated 10 or more times, preferably 50 or more times, further preferably 100 or more times, and then discharging is performed, lithium cobalt oxide that is a surface portion of the positive electrode active material particle has an O3 structure, and the electrolyte includes an imidazolium cation.

One embodiment of the present invention is a secondary battery including a positive electrode active material and an electrolyte. In the secondary battery, the positive electrode active material is lithium cobalt oxide that has an O3 structure after charging and discharging are repeated and the electrolyte includes a compound represented by General Formula (G1). In General Formula (G1) below, R¹ represents an alkyl group having 1 to 4 carbon atoms, R², R³, and R⁴ each independently represent a hydrogen atom or an alkyl group having 1 to 4 carbon atoms, and R⁵ represents an alkyl group or a main chain composed of two or more selected from C, O, Si, N, S, and P atoms. Moreover, A⁻ represents an amide-based anion represented by (CₙF₂ₙ₊₁SO₂)₂N⁻ (n is greater than or equal to 0 and less than or equal to 3).

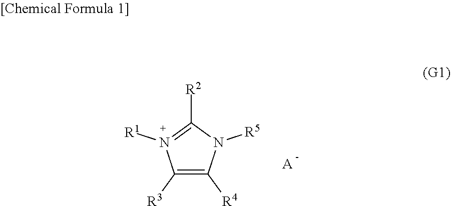

In the above structure, it is preferable that in General Formula (G1), R¹ represent one selected from a methyl group, an ethyl group, and a propyl group; one of R², R³, and R⁴ represent a hydrogen atom or a methyl group and the other two represent hydrogen atoms; R⁵ represent an alkyl group or a main chain composed of two or more selected from C, O, Si, N, S, and P atoms; and A⁻ represent any of (FSO₂)₂N⁻ and (CF₃SO₂)₂N⁻ or a mixture thereof.

In the above structure, it is preferable that in General Formula (G1), the sum of the number of carbon atoms of R¹, the number of carbon atoms of R⁵, and the number of oxygen atoms of R⁵ be 7 or less.

In the above structure, it is preferable that in General Formula (G1), R¹ represent a methyl group, R² represent a hydrogen atom, and the sum of the numbers of carbon atoms and oxygen atoms of R⁵ be 6 or less.

In the above structure, it is preferable that the electrolyte include one or more selected from a 1-butyl-3-propylimidazolium cation, a 1-ethyl-3-methylimidazolium cation, a 1-butyl-3-methylimidazolium cation, a 1-hexyl-3-methylimidazolium cation, and a 1-methyl-3-(2-propoxyethyl)imidazolium cation.

In the above structure, it is preferable that the electrolyte include a 1-ethyl-3-methylimidazolium cation.

Another embodiment of the present invention is an electronic device including the secondary battery described in any of the above, a display portion, and a sensor.

Another embodiment of the present invention is a vehicle including the secondary battery described in any of the above, an electric motor, and a control device, and the control device has a function of supplying electric power from the secondary battery to the electric motor.

### Effect of the Invention

According to one embodiment of the present invention, a lithium-ion secondary battery having high capacity and excellent charge and discharge cycle performance, and a manufacturing method thereof can be provided. According to another embodiment of the present invention, a secondary battery that can be rapidly charged, and a manufacturing method thereof can be provided. According to another embodiment of the present invention, a secondary battery having high capacity, and a manufacturing method thereof can be provided. According to another embodiment of the present invention, a secondary battery with excellent charge and discharge characteristics, and a manufacturing method thereof can be provided. According to another embodiment of the present invention, a secondary battery in which a reduction in capacity is suppressed even when a state being charged with a high voltage is held for a long time, and a manufacturing method thereof can be provided. According to another embodiment of the present invention, a highly safe or reliable secondary battery, and a manufacturing method thereof can be provided. According to another embodiment of the present invention, a secondary battery in which a reduction in capacity is suppressed even at high temperatures, and a manufacturing method thereof can be provided. According to another embodiment of the present invention, a long-life secondary battery, and a manufacturing method thereof can be provided.

According to one embodiment of the present invention, a safe, long-life, and extremely excellent secondary battery that can be rapidly charged, can be used at high temperatures, and can have a high energy density due to increased charge voltage can be provided.

According to one embodiment of the present invention, a positive electrode active material that has high capacity and excellent charge and discharge cycle performance for a lithium-ion secondary battery, and a manufacturing method thereof can be provided. A method for manufacturing a positive electrode active material with high productivity can be provided. According to one embodiment of the present invention, a positive electrode active material that suppresses a reduction in capacity in charge and discharge cycles when used for a lithium-ion secondary battery can be provided. According to one embodiment of the present invention, a positive electrode active material in which elution of a transition metal such as cobalt is suppressed even when a state being charged with a high voltage is held for a long time can be provided.

One embodiment of the present invention can provide a novel material, novel active material particles, a novel power storage device, or a manufacturing method thereof.

Note that the description of these effects does not preclude the existence of other effects. One embodiment of the present invention does not have to have all these effects. Note that effects other than these will be apparent from the description of the specification, the drawings, the claims, and the like and effects other than these can be taken from the description of the specification, the drawings, the claims, and the like.

### BRIEF DESCRIPTION OF THE DRAWINGS

FIG. 1 is a diagram illustrating crystal structures of a positive electrode active material.

FIG. 2 is a diagram illustrating crystal structures of a positive electrode active material.

FIG. 3 is a cross-sectional schematic view of a positive electrode active material particle.

FIG. 4A and FIG. 4B are diagrams illustrating examples of a method for forming a positive electrode active material of one embodiment of the present invention.

FIG. 5A to FIG. 5C are diagrams illustrating examples of a method for forming a positive electrode active material of one embodiment of the present invention.

FIG. 6 is a diagram illustrating an example of a method for forming a positive electrode active material of one embodiment of the present invention.

FIG. 7A to FIG. 7C are diagrams illustrating examples of a method for forming a positive electrode active material of one embodiment of the present invention.

FIG. 8A, FIG. 8B, FIG. 8C, and FIG. 8D are cross-sectional schematic views of negative electrode active material particles.

FIG. 9A, FIG. 9B, FIG. 9C, and FIG. 9D show an example of a cross-sectional view of a secondary battery.

FIG. 10A and FIG. 10B are diagrams showing examples of the appearances of secondary batteries.

FIG. 11A and FIG. 11B are diagrams illustrating a method for manufacturing a secondary battery.

FIG. 12A and FIG. 12B are diagrams illustrating a method for manufacturing a secondary battery.

FIG. 13 is a diagram showing an example of the appearance of a secondary battery.

FIG. 14 is a top view showing an example of a manufacturing apparatus for a secondary battery.

FIG. 15 is a cross-sectional view showing an example of a method for manufacturing a secondary battery.

FIG. 16A to FIG. 16C are perspective views showing an example of a method for manufacturing a secondary battery. FIG. 16D is a cross-sectional view corresponding to FIG. 16C.

FIG. 17A to FIG. 17F are perspective views showing an example of a method for manufacturing a secondary battery.

FIG. 18 is a cross-sectional view showing an example of a secondary battery.

FIG. 19A is a diagram showing an example of a secondary battery. FIG. 19B and FIG. 19C are diagrams showing an example of a method for fabricating a stack.

FIG. 20A to FIG. 20C are diagrams showing an example of a method for manufacturing a secondary battery.

FIG. 21A and FIG. 21B are cross-sectional views showing examples of stacks. FIG. 21C is a cross-sectional view showing an example of a secondary battery.

FIG. 22A and FIG. 22B are diagrams showing examples of secondary batteries. FIG. 22C is a diagram illustrating the internal state of a secondary battery.

FIG. 23A to FIG. 23C are diagrams showing an example of a secondary battery.

FIG. 24A is a perspective view showing an example of a battery pack. FIG. 24B is a block diagram showing an example of a battery pack. FIG. 24C is a block diagram showing an example of a vehicle including a motor.

FIG. 25A to FIG. 25E are diagrams showing examples of transport vehicles.

FIG. 26A is a diagram illustrating an electric bicycle, FIG. 26B is a diagram illustrating a secondary battery of the electric bicycle, and FIG. 26C is a diagram illustrating an electric motorcycle.

FIG. 27A and FIG. 27B are diagrams showing examples of power storage devices.

FIG. 28A to FIG. 28E are diagrams showing examples of electronic devices.

FIG. 29A to FIG. 29H are diagrams showing examples of electronic devices.

FIG. 30A to FIG. 30C are diagrams showing an example of an electronic device.

FIG. 31 is a diagram showing examples of electronic devices.

FIG. 32A to FIG. 32C are diagrams showing examples of electronic devices.

FIG. 33A to FIG. 33C are diagrams showing examples of electronic devices.

FIG. 34A and FIG. 34B are diagrams showing cycle performance of secondary batteries.

FIG. 35A and FIG. 35B are diagrams showing cycle performance of secondary batteries.

FIG. 36 is a diagram showing cycle performance of secondary batteries.

FIG. 37A to FIG. 37E show cross-sectional SEM images of a negative electrode.

FIG. 38A to FIG. 38E show cross-sectional SEM images of a negative electrode.

FIG. 39 shows the measurement results of the concentrations of cobalt and the thicknesses of a coating film of the negative electrode, which are obtained by EDX analysis.

FIG. 40A to FIG. 40D show SEM images of positive electrodes.

### MODE FOR CARRYING OUT THE INVENTION

Embodiments of the present invention will be described in detail below with reference to the drawings. Note that the present invention is not limited to the following description, and it is readily understood by those skilled in the art that modes and details of the present invention can be modified in various ways. In addition, the present invention should not be construed as being limited to the description of the embodiments below.

In this specification and the like, crystal planes and orientations are indicated by the Miller index. In the crystallography, a bar is placed over a number in the expression of crystal planes and orientations; however, in this specification and the like, because of application format limitations, crystal planes and orientations may be expressed by placing − (a minus sign) at the front of a number instead of placing a bar over the number. Furthermore, an individual direction that shows an orientation in a crystal is denoted by “[ ]”, a set direction that shows all of the equivalent orientations is denoted by “< >”, an individual plane that shows a crystal plane is denoted by “( )”, and a set plane having equivalent symmetry is denoted by “{ }”.

In this specification and the like, segregation refers to a phenomenon in which in a solid made of a plurality of elements (e.g., A, B, and C), a certain element (e.g., B) is spatially non-uniformly distributed.

In this specification and the like, a surface portion of a particle of an active material or the like refers to a region from a surface to a depth of approximately 10 nm. A plane generated by a crack may also be referred to as a surface. In addition, a region whose position is deeper than that of the surface portion is referred to as an inner portion.

In this specification and the like, a layered rock-salt crystal structure of a composite oxide containing lithium and a transition metal refers to a crystal structure in which a rock-salt ion arrangement where cations and anions are alternately arranged is included and the transition metal and lithium are regularly arranged to form a two-dimensional plane, so that lithium can be two-dimensionally diffused. Note that a defect such as a cation or anion vacancy may exist. Moreover, in the layered rock-salt crystal structure, strictly, a lattice of a rock-salt crystal is distorted in some cases.

In this specification and the like, a rock-salt crystal structure refers to a structure in which cations and anions are alternately arranged. Note that a cation or anion vacancy may exist.

In this specification and the like, an O3′ type crystal structure (also referred to as a pseudo-spinel crystal structure) of a composite oxide containing lithium and a transition metal refers to a crystal structure with a space group R-3m, which is not a spinel crystal structure but a crystal structure where oxygen is hexacoordinated to ions of cobalt, magnesium, or the like and the cation arrangement has symmetry similar to that of the spinel crystal structure. Note that in the O3′ type crystal structure, oxygen is tetracoordinated to a light element such as lithium in some cases. Also in that case, the ion arrangement has symmetry similar to that of the spinel crystal structure.

The O3′ type crystal structure can also be regarded as a crystal structure that contains Li between layers at random but is similar to a CdCl₂ type crystal structure. The crystal structure similar to the CdCl₂ type crystal structure is close to a crystal structure of lithium nickel oxide when charged up to a charge depth of 0.94 (Li_{0.06}NiO₂); however, pure lithium cobalt oxide or a layered rock-salt positive electrode active material containing a large amount of cobalt is known not to have this crystal structure in general.

Anions of a layered rock-salt crystal and anions of a rock-salt crystal have a cubic close-packed structure (face-centered cubic lattice structure). Anions of an O3′ type crystal are also presumed to have a cubic close-packed structure. When the O3′ type crystal is in contact with the layered rock-salt crystal and the rock-salt crystal, there is a crystal plane at which orientations of cubic close-packed structures composed of anions are aligned. Note that a space group of the layered rock-salt crystal and the O3′ type crystal is R-3m, which is different from a space group Fm-3m of a rock-salt crystal (a space group of a general rock-salt crystal) and a space group Fd-3m of a rock-salt crystal (a space group of a rock-salt crystal having the simplest symmetry); thus, the Miller index of the crystal plane satisfying the above conditions in the layered rock-salt crystal and the O3′ type crystal is different from that in the rock-salt crystal. In this specification, a state where the orientations of the cubic close-packed structures composed of anions in the layered rock-salt crystal, the O3′ type crystal, and the rock-salt crystal are aligned is sometimes referred to as a state where crystal orientations are substantially aligned.

A secondary battery includes a positive electrode and a negative electrode, for example. A positive electrode active material is a material included in the positive electrode. The positive electrode active material is a substance that performs a reaction contributing to the charge and discharge capacity, for example. Note that the positive electrode active material may partly contain a substance that does not contribute to the charge and discharge capacity.

In this specification and the like, the positive electrode active material of one embodiment of the present invention is expressed as a positive electrode material, a secondary battery positive electrode material, or the like in some cases. In this specification and the like, the positive electrode active material of one embodiment of the present invention preferably contains a compound. In this specification and the like, the positive electrode active material of one embodiment of the present invention preferably contains a composition. In this specification and the like, the positive electrode active material of one embodiment of the present invention preferably contains a composite.

### Embodiment 1

In this embodiment, an example of a secondary battery of one embodiment of the present invention is described.

As described in Example below, it was found that the secondary battery of one embodiment of the present invention has extremely stable characteristics even when charged with a high voltage. In addition, the secondary battery of one embodiment of the present invention can operate stably in a wide temperature range. According to one embodiment of one embodiment of the present invention, a secondary battery having significantly excellent characteristics can be achieved.

A positive electrode active material of one embodiment of the present invention is an oxide containing a metal serving as a carrier ion (hereinafter an element A) and a metal whose valence number changes due to charging and discharging of a secondary battery (hereinafter a metal M).

As the element A, one or more selected from alkali metals such as lithium, sodium, and potassium and Group 2 elements such as calcium, beryllium, and magnesium can be used, for example. The element A is preferably an element that functions as a metal serving as a carrier ion.

As the metal M, for example, a transition metal can be used. The positive electrode active material of one embodiment of the present invention contains one or more of cobalt, nickel, and manganese, particularly cobalt, as the metal M, for example. The positive electrode active material of one embodiment of the present invention may contain, as the metal M, an element that has no valence number change and can have the same valence number as the metal M, such as aluminum, specifically, a trivalent representative element, for example.

The positive electrode active material of one embodiment of the present invention may be represented by the chemical formula AM_{y}O_{z} (y>0 and z>0). Lithium cobalt oxide may be represented by LiCoO₂. Lithium nickel oxide may be represented by LiNiO₂.

The positive electrode active material of one embodiment of the present invention preferably contains an element X. An element such as magnesium, calcium, zirconium, lanthanum, barium, titanium, or yttrium can be used as the element X. An element such as nickel, aluminum, cobalt, manganese, vanadium, iron, chromium, or niobium can be used as the element X. An element such as copper, potassium, sodium, zinc, chlorine, fluorine, hafnium, silicon, sulfur, phosphorus, boron, or arsenic can be used as the element X. Two or more of the elements described above as the element X may be used in combination.

Part of the element X may substitute at the element A position, for example. Alternatively, part of the element X may substitute at the metal M position, for example.

The positive electrode active material of one embodiment of the present invention may be represented by the chemical formula A_{1-w}X_{w}M_{y}O_{z} (y>0, z>0, and 0<w<1). The positive electrode active material of one embodiment of the present invention may be represented by the chemical formula AM_{y-j}XⱼO_{z} (y>0, z>0, and 0<j<y). The positive electrode active material of one embodiment of the present invention may be represented by the chemical formula A_{1-w}X_{w}M_{y-j}XⱼO_{z} (y>0, z>0, 0<w<1, and 0<j<y).

Furthermore, the positive electrode active material of one embodiment of the present invention preferably contains halogen in addition to the element X. The positive electrode active material preferably contains halogen such as fluorine or chlorine. When the positive electrode active material of one embodiment of the present invention contains the halogen, substitution of the element X at the element A position is promoted in some cases.

As charge voltage of a secondary battery increases, the crystal structure of a positive electrode active material might become unstable and the characteristics of the secondary battery might be reduced. As an example, the case is described where a material having a layered crystal structure in which a metal A is extracted from a space between layers due to a charge reaction is used as a positive electrode active material. The increase in charge voltage of such a positive electrode active material can increase charge capacity and discharge capacity. Meanwhile, as charge voltage is increased, a larger amount of the metal A may be extracted from the positive electrode active material and a change in the crystal structure such as a change in the interlayer distance or generation of displacement of a layer may noticeably occur. In the case where a change in the crystal structure due to insertion and extraction of the metal A is irreversible, the crystal structure may be gradually broken along with repetitive charging and discharging and a noticeable reduction in capacity due to charge and discharge cycles may occur.

An increase in charge voltage may facilitate elution of the metal M contained in the positive electrode active material into an electrolyte. Elution of the metal M from the positive electrode active material into the electrolyte might decrease the amount of the metal M of the positive electrode active material and might decrease the capacity of a positive electrode.

In the positive electrode active material of one embodiment of the present invention, the metal M is mainly bonded to oxygen. Release of oxygen from the positive electrode active material might cause noticeable elution of the metal M.

As the electrolyte, a salt of a metal serving as a carrier ion and the following solvents such as carbonate are used. For example, an aprotic solvent such as ethylene carbonate (EC), propylene carbonate (PC), butylene carbonate, chloroethylene carbonate, vinylene carbonate, γ-butyrolactone, γ-valerolactone, dimethyl carbonate (DMC), diethyl carbonate (DEC), ethyl methyl carbonate (EMC), methyl formate, methyl acetate, ethyl acetate, methyl propionate, ethyl propionate, propyl propionate, methyl butyrate, 1,3-dioxane, 1,4-dioxane, dimethoxyethane (DME), dimethyl sulfoxide, diethyl ether, methyl diglyme, acetonitrile, benzonitrile, tetrahydrofuran, sulfolane, or sultone is used.

When the oxidation number of the metal M contained in the positive electrode active material becomes large in charging, the positive electrode active material has high reactivity and is brought into a state where a reaction with an organic solvent, specifically, carbonate with high polarity or the like likely occurs. For example, oxygen in the positive electrode active material is released and the organic solvent is oxidized. When oxygen is released, elution of the metal M easily occurs.

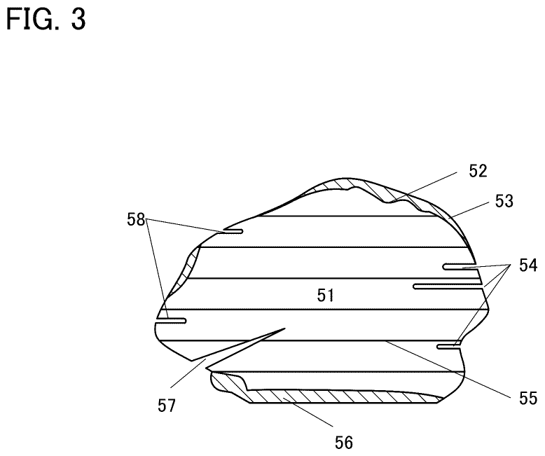

When charging and discharging are performed under high-voltage conditions at 4.5 V or higher or at a high temperature (45° C. or higher), a progressive defect (also referred to as a pit) might be generated in a positive electrode active material particle. In addition, a defect such as a crevice (also referred to as a crack) might be generated by expansion and contraction of a positive electrode active material particle due to charging and discharging. FIG. 3 shows a schematic cross-sectional view of a positive electrode active material particle 51. Although pits of the positive electrode active material particle 51 are illustrated as holes denoted by a pit 54 and a pit 58 in FIG. 3, their opening shapes are not circular but have depths, and a crack is illustrated as a crack 57 in FIG. 3. Moreover, FIG. 3 illustrates a crystal plane 55, a depression 52, and barrier films 53 and 56 as a crystal plane, a depression, and barrier films, respectively.

A positive electrode active material particle has a defect and the defect might change before and after charging and discharging. When used in a secondary battery, a positive electrode active material particle might undergo a phenomenon such as chemical or electrochemical erosion or degradation due to environmental substances (e.g., an electrolyte solution) surrounding the positive electrode active material particle. This degradation does not occur uniformly in the surface of the positive electrode active material particle but occurs locally in a concentrated manner, and a defect is formed deeply from the surface toward the inner portion, for example, by repetitive charging and discharging of the secondary battery.

Progress of a defect in a positive electrode active material particle to form a hole can be referred to as pitting corrosion, and the hole generated by this phenomenon is also referred to as a pit in this specification.

In this specification, a crack and a pit are different from each other. Immediately after formation of a positive electrode active material particle, a crack can exist but a pit does not exist. In lithium cobalt oxide, for example, a pit can also be regarded as a hole formed by extraction of some layers of cobalt and oxygen due to charging and discharging under high-voltage conditions at 4.5 V or higher or at a high temperature (45° C. or higher), i.e., a portion from which cobalt has been eluted. A crack refers to a surface newly generated by application of physical pressure or a crevice generated owing to a grain boundary. A crack might be caused by expansion and contraction of a particle due to charging and discharging. A pit might be generated from a crack or a void inside a particle.

Cobalt is eluted in lithium cobalt oxide due to charging and discharging with a high voltage or at a high temperature, whereby a crystal phase that is different from the lithium cobalt oxide may be formed in a surface portion. For example, one or more of Co₃O₄ having a spinel structure, LiCo₂O₄ having a spinel structure, and CoO having a rock-salt structure may be formed. These materials are materials having lower discharge capacity than lithium cobalt oxide or not contributing to charging and discharging, for example. Thus, formation of these materials in the surface portion might decrease the discharge capacity of the secondary battery. Furthermore, deterioration of output characteristics and deterioration of low-temperature characteristics might be caused in the secondary battery. These materials are formed in the vicinity of a pit in some cases.

The metal M is eluted from the positive electrode active material, the electrolyte transfers an ion of the metal M, and the metal M may be precipitated at the surface of a negative electrode. In addition, at the surface of the negative electrode, a coating film may be formed from the metal M and a decomposition product of the electrolyte. The formation of the coating film makes insertion and extraction of carrier ions into/from a negative electrode active material difficult, which might lead to deterioration of the rate characteristics, low-temperature characteristics, or the like of the secondary battery.

Since the positive electrode active material of one embodiment of the present invention can have an O3′ structure described later in charging, charging can be performed to a large charge depth. The increase in charge depth can increase the capacity of the positive electrode, so that the energy density of the secondary battery can be increased. Even in the case of using an extremely high charge voltage, charging and discharging can be repeated.

Note that in the case where charging is performed at a higher charge voltage, the metal M has a larger oxidation number. In such a state, elution of the metal M easily occurs as described above.

In the secondary battery of one embodiment of the present invention, elution of the metal M easily occurs due to an extremely high charge voltage, but the electrolyte containing a desired ionic liquid can suppress elution of the metal M. Thus, both a high charge voltage and suppression of elution of the metal M can be achieved. Moreover, charging and discharging at a high rate can be achieved. Furthermore, excellent charge and discharge characteristics at low temperatures can be achieved.

When a positive electrode active material layer is formed on a current collector and then pressing is performed, steps may be observed on the particle surface which is in the perpendicular direction (the c-axis direction) with respect to the lattice fringes observed in a cross-sectional STEM image or the like. In addition, a trace of deformation along the lattice fringe direction (the a-b plane direction) may be observed. A stripe pattern observed on the surface of the particle due to the steps on the surface of the particle where displacement occurs due to the pressing is referred to as a slip. A crystal structure is unstable at such a slip of the particle, which might decrease the characteristics of the secondary battery. Thus, it is desirable to reduce the number of slips of the particle or prevent generation of a slip.

The present inventors found that a secondary battery having extremely excellent characteristics can be achieved by using the positive electrode active material of one embodiment of the present invention and a desired ionic liquid having characteristics suitable for the secondary battery of one embodiment of the present invention.

The present inventors also found that in the secondary battery of one embodiment of the present invention, generation of a pit is suppressed in the positive electrode active material after repetitive charging and discharging. It was also found that in the secondary battery of one embodiment of the present invention, a heterogeneous phase does not exist or a heterogeneous phase is not substantially included in the surface portion of the positive electrode active material. Specifically, for example, it was found that in the case where the positive electrode active material is lithium cobalt oxide, Co₃O₄ having a spinel structure, LiCo₂O₄ having a spinel structure, and CoO having a rock-salt structure do not exist or are not substantially included in the surface portion of the positive electrode active material. It was also found that in the secondary battery of one embodiment of the present invention, a heterogeneous phase does not exist or a heterogeneous phase is not substantially included in the vicinity of a pit of the positive electrode active material. Specifically, for example, it was found that in the case where the positive electrode active material is lithium cobalt oxide, Co₃O₄ having a spinel structure, LiCo₂O₄ having a spinel structure, and CoO having a rock-salt structure do not exist or are not substantially included in the vicinity of a pit of the positive electrode active material. For the expression “not substantially included”, dust or the like attached to the surface is not taken into consideration, for example.

The present inventors also found that in the secondary battery of one embodiment of the present invention, after repetitive charging and discharging, a coating film on the surface of a negative electrode active material is thin and the amount of the metal M detected on the surface of the negative electrode active material or in the coating film formed on the surface of the negative electrode active material is extremely small.

It is suggested that in the secondary battery of one embodiment of the present invention, the amount of the metal M detected on the surface of the negative electrode active material or in the coating film formed on the surface of the negative electrode active material is extremely small and the coating film is thin. For this reason, it is possible to achieve a secondary battery that includes a negative electrode active material into and from which carrier ions are easily inserted and extracted, has high output characteristics, and is easily charged and discharged even at low temperatures, for example.

In the secondary battery of one embodiment of the present invention, elution of the metal M can be suppressed; thus, a reduction in capacity is suppressed and the break of a crystal structure can also be suppressed. Thus, it is possible to achieve an excellent secondary battery in which a reduction in capacity is suppressed even when the secondary battery is charged and discharged repeatedly, retained in a charged state, or retained at high temperatures.

Furthermore, in the secondary battery of one embodiment of the present invention, a heterogeneous phase is not substantially formed on the surface of the positive electrode, so that a reduction in capacity is suppressed and carrier ions are easily inserted and extracted into/from the positive electrode active material. Thus, a secondary battery in which a reduction in capacity is suppressed can be achieved. Moreover, a secondary battery that has high output characteristics and is easily charged and discharged even at low temperatures can be achieved.

An ionic liquid has low volatility and low inflammability, and is stable in a wide temperature range. An ionic liquid is not easily volatilized even at high temperatures, so that expansion of a secondary battery due to gas generated from an electrolyte solution can be suppressed. Therefore, the secondary battery stably operates even at high temperatures. Furthermore, an ionic liquid has low inflammability and is less likely to burn.

For example, the above-described organic solvent has a boiling point lower than 150° C. and has high volatility; therefore, gas might be generated when a secondary battery is used at high temperatures and an exterior body of the secondary battery might be expanded. In addition, an organic solvent has a flash point lower than or equal to 50° C. in some cases. In contrast, an ionic liquid has low volatility, and is extremely stable at up to a temperature lower than a temperature at which a reaction such as decomposition occurs, e.g., up to approximately 300° C.

Therefore, with use of an ionic liquid, a highly safe secondary battery that can be used at high temperatures can be achieved. For example, with use of an ionic liquid, a secondary battery that has stable characteristics even at 50° C. or higher, 60° C. or higher, or 80° C. or higher can be achieved.

In other words, the secondary battery of one embodiment of the present invention can favorably operate in a wide temperature range from a low temperature to a high temperature.

The secondary battery of one embodiment of the present invention can have a high charge voltage when including a positive electrode active material in which an irreversible change in a crystal structure is suppressed at a high charge voltage, so that a secondary battery with high energy density can be achieved. Moreover, in the secondary battery of one embodiment of the present invention using an ionic liquid for an electrolyte, elution of the metal M from the positive electrode active material can be suppressed; thus, a reduction in capacity due to charge and discharge cycles can be suppressed even when charging and discharging are repeated with a high charge voltage.

Here, a surface portion is preferably a region that is less than or equal to 50 nm, preferably less than or equal to 35 nm, further preferably less than or equal to 20 nm from the surface, for example. In addition, a region in a deeper position than a surface portion is referred to as an inner portion.

An ionic liquid is a salt formed by a combination of a cation and an anion. An ionic liquid is referred to as a room temperature molten salt in some cases.

By using the positive electrode active material of one embodiment of the present invention in combination with an ionic liquid, elution of the metal M from the positive electrode active material can be suppressed in the state of a large charge depth. The positive electrode active material of one embodiment of the present invention contains the element X. The element X in the positive electrode active material of one embodiment of the present invention preferably has a concentration gradient. The concentration of the element X preferably has a gradient that increases from the inner portion toward the surface. The gradient of the concentration of the element X can be evaluated using energy dispersive X-ray spectroscopy (EDX).

As described above, an ionic liquid is stable even at high temperatures. However, when other components of a secondary battery such as a positive electrode active material, a negative electrode active material, and an exterior body change at high temperatures, particularly irreversibly change, a significant decrease in the capacity of the secondary battery might occur.

For example, when the crystal structure of a material included in a positive electrode active material irreversibly changes due to charging at high temperatures, a secondary battery significantly deteriorates. For example, a significant reduction in capacity due to charge and discharge cycles might occur. The crystal structure of a positive electrode might become more unstable at higher temperatures and at a higher charge voltage.

When a positive electrode active material whose crystal structure is extremely stable at a high charge voltage and at high temperatures is used for the secondary battery of one embodiment of the present invention, excellent characteristics can be achieved even at high temperatures and at a high charge voltage, so that an ionic liquid can sufficiently exert its effect. In other words, a significant improvement in characteristics achieved by employing the structure of the secondary battery of one embodiment of the present invention is found when the structure is combined with the positive electrode active material of one embodiment of the present invention.

The positive electrode active material of one embodiment of the present invention preferably contains the element X as described later, and preferably contains halogen in addition to the element X It is suggested that when the positive electrode active material of one embodiment of the present invention contains the element X or contains halogen in addition to the element X, a reaction with an ionic liquid on the surface of the positive electrode active material is suppressed. As described above, an ionic liquid is extremely stable even at high temperatures. Meanwhile, in the secondary battery of one embodiment of the present invention, the range of reaction potential is extremely wide. In such a wide reaction potential range, a reaction with an ionic liquid on the surface of the active material is concerned in some cases. When the positive electrode active material of one embodiment of the present invention is used, a reaction with an ionic liquid is suppressed and it is suggested that a more stable secondary battery is provided.

By employing the structure of the secondary battery of one embodiment of the present invention, for example, it is possible to achieve a secondary battery that can be repeatedly charged with a high charge voltage and even at a high temperature of 42° C. or higher. For example, it is possible to achieve a secondary battery that can be repeatedly charged at an environmental temperature of 42° C. or higher with use of graphite for a negative electrode while the upper limit voltage of the charging is preferably 4.37 or higher, further preferably 4.40 V or higher, still further preferably 4.42 or higher, still further preferably 4.44 V or higher, e.g., approximately 4.45 V.

Furthermore, an excellent secondary battery can be achieved even at higher temperatures. For example, it is sometimes possible to provide a secondary battery that stably operates at higher than or equal to 42° C. and lower than or equal to 200° C., higher than or equal to 42° C. and lower than or equal to 180° C., higher than or equal to 42° C. and lower than or equal to 150° C., higher than or equal to 42° C. and lower than or equal to 120° C., higher than or equal to 42° C. and lower than or equal to 100° C., or higher than or equal to 42° C. and lower than or equal to 90° C.

The secondary battery of one embodiment of the present invention has a discharge capacity higher than or equal to 160 mAh/g after the accumulated amount of electric charge of 57000 mAh/g is discharged. Here, the discharge capacity is preferably measured at 0.2 C, for example. The accumulated amount of electric charge and discharge capacity are preferably calculated per weight of the positive electrode active material.

The secondary battery of one embodiment of the present invention that includes graphite for a negative electrode has a discharge capacity higher than or equal to 160 mAh/g after charging is performed 300 times at 25° C. and at a charge voltage of 4.5 V. Here, the discharge capacity is preferably measured at 0.2 C, for example. The accumulated amount of electric charge and discharge capacity are preferably calculated per weight of the positive electrode active material.

The secondary battery of one embodiment of the present invention is preferably used in combination with a battery control circuit. The battery control circuit preferably has a function of controlling charging, for example. Controlling charging refers to, for example, monitoring a parameter of a secondary battery and changing charge conditions in accordance with a state. Examples of a parameter to be monitored of a secondary battery include the voltage, current, temperature, amount of electric charge, and impedance of the secondary battery.

The secondary battery of one embodiment of the present invention is preferably used in combination with a sensor. The sensor preferably has a function of measuring, for example, one or more of displacement, position, speed, acceleration, angular velocity, rotational frequency, distance, light, liquid, magnetism, temperature, chemical substance, sound, time, hardness, electric field, current, voltage, electric power, radiation, flow rate, humidity, gradient, oscillation, odor, and infrared rays.

Charging of the secondary battery of one embodiment of the present invention is preferably controlled in accordance with a value measured by the sensor. An example of control of the secondary battery using a temperature sensor will be described later.

### [Positive Electrode Active Material]

A positive electrode active material that is preferably used for the secondary battery of one embodiment of the present invention will be described below.

### <Structure of Positive Electrode Active Material>

The positive electrode active material preferably contains the element A. As the element A, one or more selected from alkali metals such as lithium, sodium, and potassium and Group 2 elements such as calcium, beryllium, and magnesium can be used, for example.

In the positive electrode active material, carrier ions are extracted from the positive electrode active material due to charging. A larger amount of the extracted element A means a larger amount of ions contributing to the capacity of a secondary battery, increasing the capacity. Meanwhile, a large amount of the extracted element A easily causes the break of the crystal structure of a compound contained in the positive electrode active material. The broken crystal structure of the positive electrode active material may lead to a decrease in the discharge capacity due to charge and discharge cycles. The positive electrode active material of one embodiment of the present invention contains the element X, whereby the break of a crystal structure that would occur when carrier ions are extracted in charging of a secondary battery may be suppressed. Part of the element X substitutes at the element A position, for example. An element such as magnesium, calcium, zirconium, lanthanum, or barium can be used as the element X As another example, an element such as copper, potassium, sodium, or zinc can be used as the element X Two or more of the elements described above as the element X may be used in combination.

Furthermore, the positive electrode active material of one embodiment of the present invention preferably contains halogen in addition to the element X. The positive electrode active material preferably contains halogen such as fluorine or chlorine. When the positive electrode active material of one embodiment of the present invention contains the halogen, substitution of the element X at the element A position is promoted in some cases.

In the case where the positive electrode active material of one embodiment of the present invention contains the element X or contains halogen in addition to the element X, electric conductivity on the surface of the positive electrode active material is sometimes suppressed.

The positive electrode active material of one embodiment of the present invention contains the metal M. The metal M is a transition metal, for example. The positive electrode active material of one embodiment of the present invention contains one or more of cobalt, nickel, and manganese, particularly cobalt, as the metal M, for example. The positive electrode active material may contain, at the metal M position, an element that has no valence number change and can have the same valence number as the metal M, such as aluminum, specifically, a trivalent representative element, for example. The above-described element X may substitute at the metal M position, for example. In the case where the positive electrode active material of one embodiment of the present invention is an oxide, the element X may substitute at the oxygen position.

As the positive electrode active material of one embodiment of the present invention, a lithium composite oxide having a layered rock-salt crystal structure is preferably used, for example. Specifically, as the lithium composite oxide having a layered rock-salt crystal structure, lithium cobalt oxide, lithium nickel oxide, a lithium composite oxide containing nickel, manganese, and cobalt, or a lithium composite oxide containing nickel, cobalt, and aluminum can be used, for example. Moreover, such a positive electrode active material is preferably represented by a space group R-3m.

In the positive electrode active material having a layered rock-salt crystal structure, increasing the charge depth may cause the break of a crystal structure. Here, the break of a crystal structure refers to displacement of a layer, for example. In the case where the break of a crystal structure is irreversible, the capacity of a secondary battery might be decreased by repetitive charging and discharging.

The positive electrode active material of one embodiment of the present invention includes the element X, whereby the displacement of a layer can be suppressed even when the charge depth is increased, for example. By suppressing the displacement, a change in volume due to charging and discharging can be small. Accordingly, the positive electrode active material of one embodiment of the present invention can achieve excellent cycle performance. In addition, the positive electrode active material of one embodiment of the present invention can have a stable crystal structure in a high voltage charged state. Thus, in the positive electrode active material of one embodiment of the present invention, a short circuit is unlikely to occur while the high voltage charged state is maintained, in some cases. This is preferable because the safety is further improved.

The positive electrode active material of one embodiment of the present invention has a small crystal-structure change and a small volume difference per the same number of atoms of the transition metal between a sufficiently discharged state and a high-voltage charged state.

The positive electrode active material of one embodiment of the present invention may be represented by the chemical formula AM_{y}O_{z} (y>0 and z>0). For example, lithium cobalt oxide may be represented by LiCoO₂. As another example, lithium nickel oxide may be represented by LiNiO₂.

When the charge depth is greater than or equal to 0.8, the positive electrode active material of one embodiment of the present invention, which contains the element X, may have a structure that is represented by the space group R-3m and is not a spinel crystal structure but is a structure where oxygen is hexacoordinated to ions of the metal M (e.g., cobalt), the element X (e.g., magnesium), and the like and the cation arrangement has symmetry similar to that of the spinel crystal structure. This structure is referred to as the O3′ type crystal structure in this specification and the like. Note that in the O3′ type crystal structure, oxygen is tetracoordinated to a light element such as lithium in some cases. Also in that case, the ion arrangement has symmetry similar to that of the spinel crystal structure.

Extraction of carrier ions due to charging makes the structure of a positive electrode active material unstable. The O3′ type crystal structure is said to be a structure that can maintain high stability in spite of extraction of carrier ions.

The O3′ type crystal structure can also be regarded as a crystal structure that contains Li between layers at random but is similar to a CdCl₂ type crystal structure. The crystal structure similar to the CdCl₂ type crystal structure is close to a crystal structure of lithium nickel oxide when charged up to a charge depth of 0.94 (Li_{0.06}NiO₂); however, pure lithium cobalt oxide or a layered rock-salt positive electrode active material containing a large amount of cobalt is known not to have this crystal structure in general.

Anions of a layered rock-salt crystal and anions of a rock-salt crystal have a cubic close-packed structure (face-centered cubic lattice structure). Anions of an O3′ type crystal are also presumed to have a cubic close-packed structure. When the O3′ type crystal is in contact with the layered rock-salt crystal and the rock-salt crystal, there is a crystal plane at which orientations of cubic close-packed structures composed of anions are aligned. Note that a space group of the layered rock-salt crystal and the O3′ type crystal is R-3m, which is different from the space group Fm-3m of a rock-salt crystal (a space group of a general rock-salt crystal) and the space group Fd-3m of a rock-salt crystal (a space group of a rock-salt crystal having the simplest symmetry); thus, the Miller index of the crystal plane satisfying the above conditions in the layered rock-salt crystal and the O3′ type crystal is different from that in the rock-salt crystal. In this specification, a state where the orientations of the cubic close-packed structures composed of anions in the layered rock-salt crystal, the O3′ type crystal, and the rock-salt crystal are aligned is sometimes referred to as a state where crystal orientations are substantially aligned.

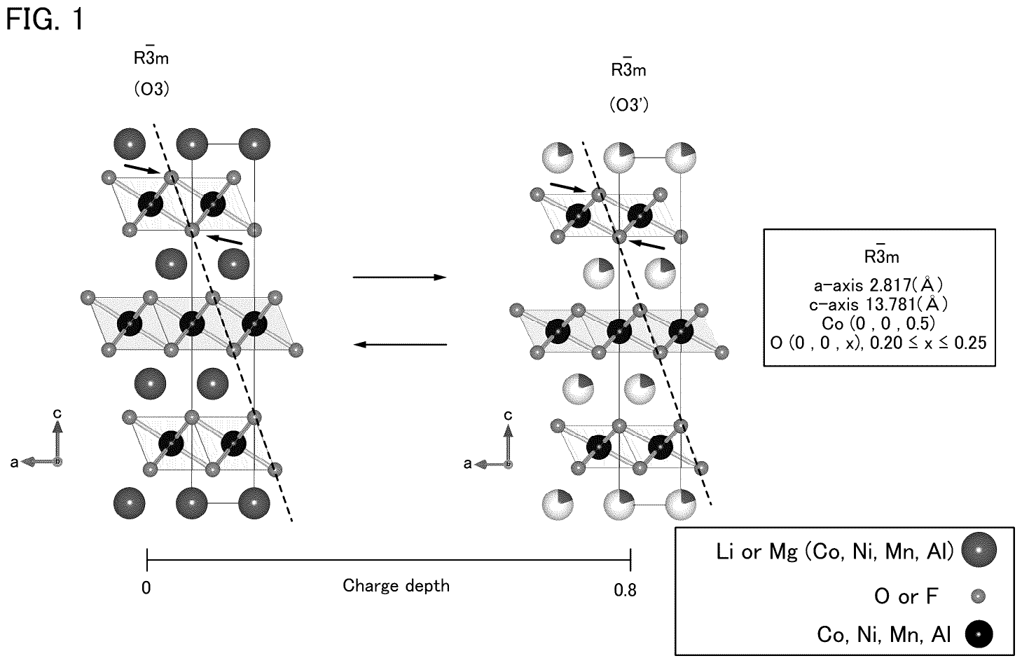

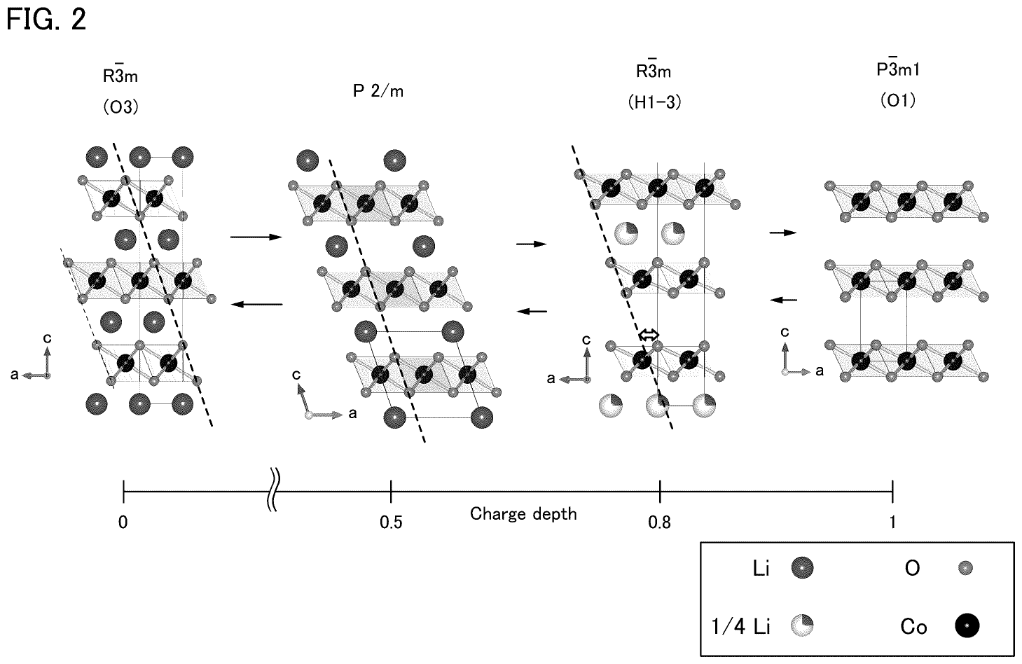

The crystal structure with a charge depth of 0 (in the discharged state) in FIG. 1 is R-3m (O3) as in FIG. 2. Meanwhile, the positive electrode active material of one embodiment of the present invention illustrated in FIG. 1 with a charge depth in a sufficiently charged state includes a crystal whose structure is different from the H1-3 type crystal structure illustrated in FIG. 2 (the space group R-3m). This structure belongs to the space group R-3m and is not the spinel crystal structure but has symmetry in cation arrangement similar to that of the spinel structure because an ion of cobalt, magnesium, or the like occupies a site coordinated to six oxygen atoms. Furthermore, the symmetry of CoO₂ layers of this structure is the same as that in the O3 type crystal structure. Accordingly, this structure is referred to as an O3′ type crystal structure or a pseudo-spinel crystal structure in this specification and the like. Note that although lithium exists in any of lithium sites at an approximately 20% probability in the diagram of the O3′ type crystal structure illustrated in FIG. 1, the structure is not limited thereto. Lithium may exist in only some certain lithium sites. In addition, in both the O3 type crystal structure and the O3′ type crystal structure, a slight amount of magnesium preferably exists between the CoO₂ layers, i.e., in lithium sites. In addition, a slight amount of halogen such as fluorine preferably exists at random in oxygen sites.

Note that in the O3′ type crystal structure, oxygen is tetracoordinated to a light element such as lithium in some cases. Also in that case, the ion arrangement has symmetry similar to that of the spinel crystal structure.

The O3′ type crystal structure can also be regarded as a crystal structure that contains Li between layers at random but is similar to a CdCl₂ type crystal structure. The crystal structure similar to the CdCl₂ type crystal structure is close to a crystal structure of lithium nickel oxide when charged up to a charge depth of 0.94 (Li_{0.06}NiO₂); however, pure lithium cobalt oxide or a layered rock-salt positive electrode active material containing a large amount of cobalt is known not to have this crystal structure in general.

Anions of a layered rock-salt crystal and anions of a rock-salt crystal have a cubic close-packed structure (face-centered cubic lattice structure). Anions of an O3′ type crystal are also presumed to have a cubic close-packed structure. When the O3′ type crystal is in contact with the layered rock-salt crystal and the rock-salt crystal, there is a crystal plane at which orientations of cubic close-packed structures composed of anions are aligned. Note that a space group of the layered rock-salt crystal and the O3′ type crystal is R-3m, which is different from the space group Fm-3m of a rock-salt crystal (a space group of a general rock-salt crystal) and the space group Fd-3m of a rock-salt crystal (a space group of a rock-salt crystal having the simplest symmetry); thus, the Miller index of the crystal plane satisfying the above conditions in the layered rock-salt crystal and the O3′ type crystal is different from that in the rock-salt crystal. In this specification, a state where the orientations of the cubic close-packed structures composed of anions in the layered rock-salt crystal, the O3′ type crystal, and the rock-salt crystal are aligned is sometimes referred to as a state where crystal orientations are substantially aligned.

In the positive electrode active material of one embodiment of the present invention, a change in the crystal structure when the positive electrode active material is charged with a high voltage and a large amount of lithium is extracted is inhibited as compared with a comparative example described later. As shown by dotted lines in FIG. 1, for example, CoO₂ layers hardly deviate in the crystal structures.

More specifically, the structure of the positive electrode active material of one embodiment of the present invention is highly stable even when charge voltage is high. For example, an H1-3 type crystal structure is formed at a voltage of approximately 4.6 V with the potential of a lithium metal as the reference in the positive electrode active material illustrated in FIG. 2 as an example; however, the positive electrode active material of one embodiment of the present invention can maintain the crystal structure of R-3m (O3) even at the charge voltage of approximately 4.6 V. Even at higher charge voltages, e.g., a voltage of approximately 4.65 V to 4.7 V with the potential of a lithium metal as the reference, the positive electrode active material of one embodiment of the present invention can have the O3′ type crystal structure. At charge voltage increased to be higher than 4.7 V, an H1-3 type crystal may be finally observed in the positive electrode active material of one embodiment of the present invention. In addition, the positive electrode active material of one embodiment of the present invention can have the O3′ type crystal structure even at a lower charge voltage (e.g., a charge voltage of higher than or equal to 4.5 V and lower than 4.6 V with the potential of a lithium metal as the reference).

Note that in the case where graphite is used as a negative electrode active material in a secondary battery, for example, the voltage of the secondary battery is lower than the above-mentioned voltages by the potential of graphite. The potential of graphite is approximately 0.05 V to 0.2 V with the potential of a lithium metal as the reference. Thus, even in a secondary battery which includes graphite as a negative electrode active material and which has a voltage of higher than or equal to 4.3 V and lower than or equal to 4.5 V, for example, the positive electrode active material of one embodiment of the present invention can maintain the crystal structure belonging to R-3m (O3) and moreover, can have the O3′ type structure at higher voltages, e.g., a voltage of the secondary battery of higher than 4.5 V and lower than or equal to 4.6 V. In addition, the positive electrode active material of one embodiment of the present invention can have the O3′ structure at lower charge voltages, e.g., at a voltage of the secondary battery of higher than or equal to 4.2 V and lower than 4.3 V, in some cases.

Thus, in the positive electrode active material of one embodiment of the present invention, the crystal structure is less likely to be broken even when charging and discharging are repeated at high voltage.

In addition, in the positive electrode active material of one embodiment of the present invention, a difference in the volume per unit cell between the O3 type crystal structure with a charge depth of 0 and the O3′ type crystal structure with a charge depth of 0.8 is less than or equal to 2.5%, specifically, less than or equal to 2.2%.

Note that in the unit cell of the O3′ type crystal structure, the coordinates of cobalt and oxygen can be represented by Co (0, 0, 0.5) and O (0, 0, x) within the range of 0.20×0.25.

A slight amount of magnesium existing between the CoO₂ layers, i.e., in lithium sites at random, has an effect of inhibiting displacement of the CoO₂ layers in high-voltage charging. Thus, magnesium between the CoO₂ layers makes it easier to obtain the O3′ type crystal structure.

However, cation mixing occurs when the heat treatment temperature is excessively high; thus, magnesium is highly likely to enter cobalt sites. Magnesium in the cobalt sites is less effective in maintaining the R-3m structure in high-voltage charging in some cases. Furthermore, heat treatment at an excessively high temperature might have an adverse effect; for example, cobalt might be reduced to have a valence of two or lithium might be evaporated.

In view of the above, a halogen compound such as a fluorine compound is preferably added to lithium cobalt oxide before the heat treatment for distributing magnesium throughout the particle. The addition of the halogen compound decreases the melting point of lithium cobalt oxide. The decreased melting point makes it easier to distribute magnesium throughout the particle at a temperature at which the cation mixing is unlikely to occur. Furthermore, the fluorine compound probably increases corrosion resistance to hydrofluoric acid generated by decomposition of an electrolyte solution.

When the magnesium concentration is higher than or equal to a desired value, the effect of stabilizing a crystal structure becomes small in some cases. This is probably because magnesium enters the cobalt sites in addition to the lithium sites. The number of magnesium atoms in the positive electrode active material formed by one embodiment of the present invention is preferably 0.001 times or more and 0.1 times or less, further preferably more than 0.01 times and less than 0.04 times, still further preferably approximately 0.02 as large as the number of cobalt atoms. The magnesium concentration described here may be a value obtained by element analysis on the whole particles of the positive electrode active material using ICP-MS or the like, or may be a value based on the ratio of the raw materials mixed in the process of forming the positive electrode active material, for example.

The number of nickel atoms in the positive electrode active material of one embodiment of the present invention is preferably 7.5% or lower, preferably 0.05% or higher and 4% or lower, further preferably 0.1% or higher and 2% or lower of the number of cobalt atoms. The nickel concentration described here may be a value obtained by element analysis on the whole particles of the positive electrode active material using ICP-MS or the like, or may be a value based on the ratio of the raw materials mixed in the process of forming the positive electrode active material, for example.

### <Particle Diameter>

When the particle diameter of the positive electrode active material of one embodiment of the present invention is too large, there are problems such as difficulty in lithium diffusion and too much surface roughness of an active material layer at the time when the material is applied to a current collector. By contrast, when the particle diameter is too small, there are problems such as difficulty in loading of the active material layer at the time when the material is applied to the current collector and overreaction with the electrolyte solution. Therefore, an average particle diameter (D50, also referred to as median diameter) is preferably greater than or equal to 1 μm and less than or equal to 100 μm, further preferably greater than or equal to 2 μm and less than or equal to 40 μm, still further preferably greater than or equal to 5 μm and less than or equal to 30 μm.

### <Analysis Method>

Whether or not a positive electrode active material has the O3′ type crystal structure when charged with a high voltage can be determined by analyzing a high-voltage charged positive electrode using XRD, electron diffraction, neutron diffraction, electron spin resonance (ESR), nuclear magnetic resonance (NMR), or the like. XRD is particularly preferable because the symmetry of a transition metal such as cobalt contained in the positive electrode active material can be analyzed with high resolution, the degrees of crystallinity and the crystal orientations can be compared, the distortion of lattice periodicity and the crystallite size can be analyzed, and a positive electrode itself obtained by disassembling a secondary battery can be measured with sufficient accuracy, for example.

As described above, the positive electrode active material of one embodiment of the present invention features a small change in the crystal structure between a high-voltage charged state and a discharged state. A material 50 wt % or more of which has the crystal structure that largely changes between a high voltage charged state and a discharged state is not preferable because the material cannot withstand charging and discharging with a high voltage. In addition, it should be noted that an objective crystal structure is not obtained in some cases only by addition of impurity elements. For example, although the positive electrode active material that is lithium cobalt oxide containing magnesium and fluorine is a commonality, the positive electrode active material has the O3′ type crystal structure at 60 wt % or more in some cases, and has the H1-3 type crystal structure at 50 wt % or more in other cases, when charged with a high voltage. In some cases, lithium cobalt oxide containing magnesium and fluorine may have the O3′ type crystal structure at almost 100 wt % at a predetermined voltage, and increasing the voltage to be higher than the predetermined voltage may cause the H1-3 type crystal structure. Thus, the crystal structure of the positive electrode active material of one embodiment of the present invention is preferably analyzed by XRD or the like. The combination with XRD measurement or the like enables more detailed analysis.

However, the crystal structure of a positive electrode active material in a high voltage charged state or a discharged state may be changed with exposure to the air. For example, the O3′ type crystal structure changes into the H1-3 type crystal structure in some cases. Thus, all samples are preferably handled in an inert atmosphere such as an atmosphere containing argon.

### COMPARATIVE EXAMPLE

A positive electrode active material illustrated in FIG. 2 is lithium cobalt oxide (LiCoO₂) to which either halogen or magnesium is not added in a formation method described later. The crystal structure of the lithium cobalt oxide illustrated in FIG. 2 is changed depending on a charge depth.

As illustrated in FIG. 2, lithium cobalt oxide with a charge depth of 0 (in the discharged state) includes a region having a crystal structure belonging to the space group R-3m, and includes three CoO₂ layers in a unit cell. Thus, this crystal structure is referred to as an O3 type crystal structure in some cases. Note that the CoO₂ layer has a structure in which an octahedral structure with cobalt coordinated to six oxygen atoms continues on a plane in an edge-shared state.

Lithium cobalt oxide with a charge depth of 1 has the crystal structure belonging to the space group P-3m1 and includes one CoO₂ layer in a unit cell. Hence, this crystal structure is referred to as an 01 type crystal structure in some cases.

Lithium cobalt oxide with a charge depth of approximately 0.8 has the crystal structure belonging to the space group R-3m. This structure can also be regarded as a structure in which CoO₂ structures such as a structure belonging to P-3m1 (O1) and LiCoO₂ structures such as a structure belonging to R-3m (O3) are alternately stacked. Thus, this crystal structure is referred to as an H1-3 type crystal structure in some cases. Note that the number of cobalt atoms per unit cell in the actual H1-3 type crystal structure is twice that in other structures. However, in this specification including FIG. 2, the c-axis of the H1-3 type crystal structure is half that of the unit cell for easy comparison with the other structures.

For the H1-3 type crystal structure, the coordinates of cobalt and oxygen in the unit cell can be expressed as follows, for example: Co (0, 0, 0.42150±0.00016), O₁ (0, 0, 0.27671±0.00045), and O₂ (0, 0, 0.11535±0.00045). O₁ and O₂ are each an oxygen atom. In this manner, the H1-3 type crystal structure is represented by a unit cell containing one cobalt and two oxygen. Meanwhile, the O3′ type crystal structure of one embodiment of the present invention is preferably represented by a unit cell containing one cobalt and one oxygen. This means that the symmetry of cobalt and oxygen differs between the O3′ type crystal structure and the H1-3 type structure, and the amount of change from the O3 structure is smaller in the O3′ type crystal structure than in the H1-3 type structure. A preferred unit cell for representing a crystal structure in a positive electrode active material can be selected such that the value of GOF (good of fitness) is smaller in Rietveld analysis of XRD, for example.

When charging at a high voltage of 4.6 V or higher based on the redox potential of a lithium metal or charging at a large charge depth of 0.8 or more and discharging are repeated, a change in the crystal structure of lithium cobalt oxide between the R-3m (O3) structure in a discharged state and the H1-3 type crystal structure (i.e., an unbalanced phase change) occurs repeatedly.

However, there is a large shift in the CoO₂ layers between these two crystal structures. As indicated by dotted lines and an arrow in FIG. 2, the CoO₂ layer in the H1-3 type crystal structure greatly shifts from that in R-3m (O3). Such a dynamic structural change can adversely affect the stability of the crystal structure.

A difference in volume is also large. The O3 type crystal structure in a discharged state and the H1-3 type crystal structure that contain the same number of cobalt atoms have a difference in volume of 3.0% or more.

In addition, a structure in which CoO₂ layers are arranged continuously, such as P-3m1 (O1), included in the H1-3 type crystal structure is highly likely to be unstable.

Thus, the repeated high-voltage charging and discharging breaks the crystal structure of lithium cobalt oxide. The broken crystal structure triggers deterioration of the cycle performance. This is because the broken crystal structure has a smaller number of sites where lithium can exist stably and makes it difficult to insert and extract lithium.

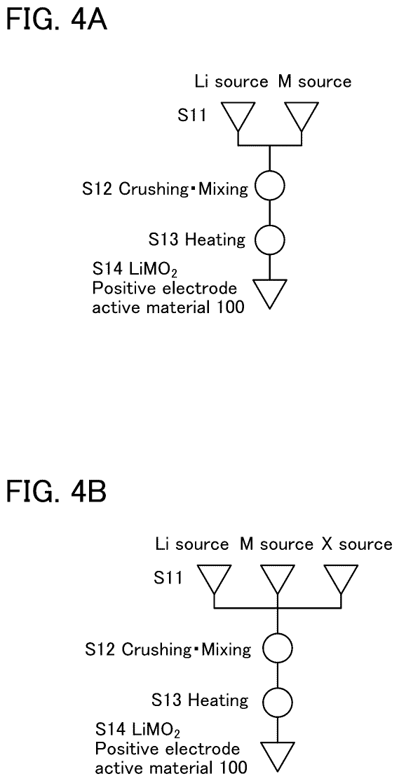

Examples of a method for forming the positive electrode active material of one embodiment of the present invention is described with reference to FIG. 4 to FIG. 7. Here, as an example, a method for forming a positive electrode active material containing lithium, a transition metal, and the element X will be described.

### [Formation Method 1 of Positive Electrode Active Material]

### <Step S11>

In Step S11 in FIG. 4A, a lithium source and a transition metal source are prepared as materials for lithium and a transition metal. Note that the transition metal source is shown as an M source in the drawing.

As the lithium source, lithium carbonate or lithium fluoride can be used, for example.

For example, at least one of manganese, cobalt, and nickel can be used as the transition metal source. As the transition metal source, cobalt alone; nickel alone; two elements of cobalt and manganese; two elements of cobalt and nickel; or three elements of cobalt, manganese, and nickel may be used, for example.

As the transition metal source used in synthesis, a high-purity material is preferably used. Specifically, the purity of the material is higher than or equal to 3N (99.9%), preferably higher than or equal to 4N (99.99%), further preferably higher than or equal to 4N5 (99.995%), still further preferably higher than or equal to 5N (99.999%). The use of a high-purity material can increase the capacity of a secondary battery and/or increase the reliability of a secondary battery.

In addition, it is preferred that the transition metal source here have high crystallinity. For example, the transition metal source preferably includes single crystal particles. The crystallinity of the transition metal source can be evaluated from a TEM (transmission electron microscopy) image, a STEM (scanning transmission electron microscopy) image, a HAADF-STEM (high-angle annular dark-field scanning transmission electron microscopy) image, an ABF-STEM (annular bright-field scanning transmission electron microscopy) image, or the like. For evaluation of the crystallinity of the transition metal source, XRD, electron diffraction, neutron diffraction, and the like can also be used. Note that the above evaluation of crystallinity can also be employed to evaluate the crystallinity of a primary particle or a secondary particle other than the transition metal source.

When metals that can form a layered rock-salt composite oxide are used, cobalt, manganese, and nickel are preferably mixed at the ratio at which the composite oxide can have a layered rock-salt crystal structure. In addition, an additive element X may be added to these transition metals as long as the composite oxide can have a layered rock-salt crystal structure. FIG. 4B shows an example of a step of adding the additive element X The lithium source, the transition metal source, and an additive element X source are prepared in Step S11, and then Step S12 is performed.

As the additive element X, one or more selected from magnesium, calcium, zirconium, lanthanum, barium, titanium, yttrium, nickel, aluminum, cobalt, manganese, vanadium, iron, chromium, niobium, copper, potassium, sodium, zinc, chlorine, fluorine, hafnium, silicon, sulfur, phosphorus, boron, and arsenic can be used. In addition to the above elements, bromine and beryllium may be used as the additive elements X Note that the additive elements Xgiven earlier are more suitable because bromine and beryllium are elements having toxicity to living things.

As the transition metal source, an oxide or a hydroxide of the metal described as an example of the transition metal, or the like can be used. As a cobalt source, cobalt oxide, cobalt hydroxide, or the like can be used.

As a manganese source, manganese oxide, manganese hydroxide, or the like can be used. As a nickel source, nickel oxide, nickel hydroxide, or the like can be used. As an aluminum source, aluminum oxide, aluminum hydroxide, or the like can be used.

### <Step S12>

Next, in Step S12, the lithium source, the transition metal source, and the additive element X source are crushed and mixed. The crushing and mixing can be performed by a dry method or a wet method. Specifically, it is preferable to use super dehydrated acetone whose moisture content is less than or equal to 10 ppm and whose purity is greater than or equal to 99.5% for crushing. Note that in this specification and the like, the term crushing can be rephrased as grinding. For the mixing, a ball mill, a bead mill, or the like can be used, for example. When a ball mill is used, zirconia balls are preferably used as media, for example. When a ball mill, a bead mill, or the like is used, the peripheral speed is preferably greater than or equal to 100 mm/s and less than or equal to 2000 mm/s in order to inhibit contamination from the media or the material. For example, mixing may be performed at a peripheral speed of 838 mm/s (the number of rotations: 400 rpm, the ball mill diameter: 40 mm). By using the above-described dehydrated acetone for the crushing and mixing, impurities that might enter the material can be reduced.

### <Step S13>

Next, in Step S13, the materials mixed in the above manner are heated. The heating in this step is preferably performed at higher than or equal to 800° C. and lower than 1100° C., further preferably at higher than or equal to 900° C. and lower than or equal to 1000° C., still further preferably at approximately 950° C. An excessively low temperature might lead to insufficient decomposition and melting of the lithium source and the transition metal source. An excessively high temperature might lead to a defect due to evaporation of lithium from the lithium source and/or excessive reduction of the metal used as the transition metal source, for example. The use of cobalt as the transition metal, for example, may lead to a defect in which cobalt has divalence.

For example, the heating time can be longer than or equal to 1 hour and shorter than or equal to 100 hours, and is preferably longer than or equal to 2 hours and shorter than or equal to hours. The heating is preferably performed in an atmosphere with little water, such as dry air (e.g., the dew point is lower than or equal to −50° C., and the dew point is further preferably lower than or equal to −80° C.). For example, the heat treatment may be performed in an atmosphere with a dew point of −93° C. Furthermore, it is suitable to perform the heating in an atmosphere where the concentrations of impurities, CH₄, CO, CO₂, and Hz, are each less than or equal to 5 ppb (parts per billion), in which case impurities can be inhibited from entering the materials.

For example, in the case where the heating is performed at 1000° C. for 10 hours, it is preferable that the temperature rising rate be 200° C./h and the flow rate of dry air be 10 L/min. After that, the heated materials can be cooled to room temperature. The temperature decreasing time from the specified temperature to room temperature is preferably longer than or equal to 10 hours and shorter than or equal to 50 hours, for example. Note that the cooling to room temperature in Step S13 is not essential.

Note that a crucible used in the heating in Step S13 is suitably made of a material into which impurities do not enter. For example, a crucible made of alumina with a purity of 99.9% may be used.

It is suitable to collect the materials subjected to the heating in Step S13 after the materials are transferred from the crucible to a mortar because impurities are prevented from entering the materials. The mortar is suitably made of a material into which impurities do not enter. Specifically, it is suitable to use a mortar made of alumina with a purity of 90% or higher, preferably 99% or higher. Note that conditions equivalent to those in Step S13 can be employed in an after-mentioned heating step other than Step S13.

### <Step S14>

Through the above steps, the positive electrode active material 100 of one embodiment of the present invention can be formed (Step S14). The positive electrode active material 100 is sometimes referred to as a composite oxide containing lithium, the transition metal, and oxygen (LiMO₂). Note that the positive electrode active material of one embodiment of the present invention only needs to have a crystal structure of a lithium composite oxide represented by LiMO₂, and the composition is not strictly limited to Li:M:O=1:1:2.

A positive electrode active material is formed using a high-purity material for the transition metal source used in synthesis and using a process which hardly allows entry of impurities in the synthesis, whereby a material that has a low impurity concentration, in other words, is highly purified can be obtained. Moreover, the positive electrode active material obtained by such a method for forming a positive electrode active material is a material having high crystallinity. With the positive electrode active material obtained by the method for forming the positive electrode active material of one embodiment of the present invention, the capacity of a secondary battery can be increased and/or the reliability of a secondary battery can be increased.

### [Formation Method 2 of Positive Electrode Active Material]

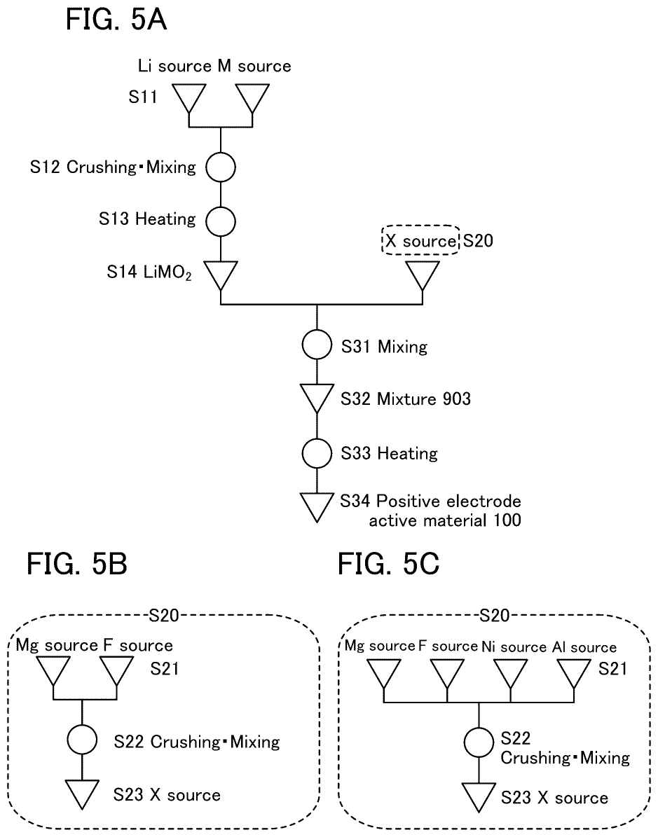

Next, another example of a method for forming a positive electrode active material of one embodiment of the present invention will be described with reference to FIG. 5A, FIG. 5B, and FIG. 5C.

In FIG. 5A, Steps S11 to S14 are performed as in FIG. 4A to prepare a composite oxide containing lithium, a transition metal, and oxygen (LiMO₂).

Note that a pre-synthesized composite oxide may be used in Step S14. In that case, Step S11 to Step S13 can be omitted. In the case where a pre-synthesized composite oxide is prepared, a high-purity material is preferably used. The purity of the material is higher than or equal to 99.5%, preferably higher than or equal to 99.9%, further preferably higher than or equal to 99.99%.

Note that a step of performing heating may be provided between Step S14 and the following Step S20. The heating can make a surface of the composite oxide smooth, for example. For the heating, the conditions that are the same as the atmosphere and temperature for Step S33 described later are used and the treatment time is shorter than that for Step S33. Having a smooth surface refers to a state where the composite oxide has little unevenness and is rounded as a whole and its corner portion is rounded. A smooth surface also refers to a surface to which few foreign matters are attached. Foreign matters are deemed to cause unevenness and are preferably not attached to a surface.

### <Step S20>

In Step S20 in FIG. 5A, an additive element X source is prepared. As the additive element X source, the above-described material can be used. A plurality of elements may be used as the additive elements X The case where a plurality of elements are used as the additive elements X is described with reference to FIG. 5B and FIG. 5C. For the addition of the additive element X, a solid phase method, a liquid phase method such as a sol-gel method, a sputtering method, an evaporation method, a CVD (chemical vapor deposition) method, a PLD (pulsed laser deposition) method, and the like can be used.

### <Step S21>

In Step S21 in FIG. 5B, a magnesium source (Mg source) and a fluorine source (F source) are prepared. In addition, a lithium source may be prepared together with the magnesium source and the fluorine source.

As the magnesium source, for example, magnesium fluoride, magnesium oxide, magnesium hydroxide, or magnesium carbonate can be used.

As the fluorine source, for example, lithium fluoride (LiF), magnesium fluoride (MgF₂), aluminum fluoride (AlF₃), titanium fluoride (TiF₄), cobalt fluoride (CoF₂ and CoF₃), nickel fluoride (NiF₂), zirconium fluoride (ZrF₄), vanadium fluoride (VF₅), manganese fluoride, iron fluoride, chromium fluoride, niobium fluoride, zinc fluoride (ZnF₂), calcium fluoride (CaF₂), sodium fluoride (NaF), potassium fluoride (KF), barium fluoride (BaF₂), cerium fluoride (CeF₂), lanthanum fluoride (LaF₃), or sodium aluminum hexafluoride (Na₃AlF₆) can be used. The fluorine source is not limited to a solid, and for example, fluorine (F₂), carbon fluoride, sulfur fluoride, oxygen fluoride (OF₂, O₂F₂, O₃F₂, O₄F₂, or O₂F), or the like may be used and mixed in the atmosphere in a heating step described later. A plurality of fluorine sources may be mixed to be used. Among them, lithium fluoride, which has a relatively low melting point of 848° C., is preferable because it is easily melted in a heating step described later.

As the lithium source, for example, lithium fluoride or lithium carbonate can be used. That is, lithium fluoride can be used as both the lithium source and the fluorine source. In addition, magnesium fluoride can be used as both the fluorine source and the magnesium source.

In this embodiment, lithium fluoride LiF is prepared as the fluorine source, and magnesium fluoride MgF₂ is prepared as the fluorine source and the magnesium source. When lithium fluoride LiF and magnesium fluoride MgF₂ are mixed at approximately LiF:MgF₂=65:35 (molar ratio), the effect of reducing the melting point becomes the highest (Non-Patent Document 4). On the other hand, when the amount of lithium fluoride increases, cycle performance might deteriorate because of too large an amount of lithium. Therefore, the molar ratio of lithium fluoride LiF to magnesium fluoride MgF₂ is preferably LiF:MgF₂=x:1 (0≤x≤1.9), further preferably LiF:MgF₂=x:1 (0.1×0.5), still further preferably LiF:MgF₂=x:1 (x=0.33 and the neighborhood thereof). Note that in this specification and the like, the vicinity means a value greater than 0.9 times and less than 1.1 times a certain value.

In the case where the following mixing and crushing step is performed by a wet method, a solvent is prepared. As the solvent, it is preferable to use a protic solvent that hardly reacts with lithium, e.g., ketone such as acetone, alcohol such as ethanol or isopropanol, ether, dioxane, acetonitrile, or N-methyl-2-pyrrolidone (NMP).

### <Step S22>

Next, in Step S22 in FIG. 5B, the above-described materials are mixed and crushed. Although the mixing can be performed by a dry method or a wet method, a wet method is preferable because the materials can be crushed to a smaller size. For example, a ball mill, a bead mill, or the like can be used for the mixing. When a ball mill is used, zirconia balls are preferably used as media, for example. Conditions of the ball mill or the bead mill may be similar to those in Step S12.

### <Step S23>

Next, in Step S23, the crushed and mixed materials are collected to obtain the additive element X source. Note that the additive element X source shown in Step S23 is formed using a plurality of materials and can be referred to as a mixture.

For example, D50 (median diameter) of the mixture is preferably greater than or equal to 600 nm and less than or equal to 20 μm, further preferably greater than or equal to 1 μm and less than or equal to 10 μm. When mixed with a composite oxide containing lithium, the transition metal, and oxygen in the later step, the mixture pulverized to such a small size is easily attached to surfaces of composite oxide particles uniformly. The mixture is preferably attached to the surfaces of the composite oxide particles uniformly because both halogen and magnesium are easily distributed to the vicinity of the surface of the composite oxide particle after heating. When there is a region containing neither halogen nor magnesium in the vicinity of the surface, the positive electrode active material might be less likely to have an O3′ type crystal structure, which is described later, in the charged state.

Note that a method in which two kinds of materials are mixed in Step S21 is shown in FIG. 5B, but one embodiment of the present invention is not limited thereto. For example, as shown in FIG. 5C, four kinds of materials (a magnesium source (Mg source), a fluorine source (F source), a nickel source (Ni source), and an aluminum source (Al source)) may be mixed to prepare the additive element X source. Alternatively, a single material, that is, one kind of material may be used to prepare the additive element X source. Note that as a nickel source, nickel oxide, nickel hydroxide, or the like can be used. As an aluminum source, aluminum oxide, aluminum hydroxide, or the like can be used.

### <Step S31>

Next, in Step S31 in FIG. 5A, LiMO₂ obtained in Step S14 and the additive element X source are mixed. The ratio of the number M of the transition metal atoms in the composite oxide containing lithium, the transition metal, and oxygen to the number Mg of magnesium atoms contained in the additive element X source is preferably M:Mg=100:y (0.1≤y≤6), further preferably M:Mg=100:y (0.3≤y≤3).

The conditions of the mixing in Step S31 are preferably milder than those of the mixing in Step S12 in order not to damage the particles of the composite oxide. For example, conditions with a lower rotation frequency or shorter time than the mixing in Step S12 are preferable. In addition, it can be said that the dry method has a milder condition than the wet method. For example, a ball mill, a bead mill, or the like can be used for the mixing. When a ball mill is used, zirconia balls are preferably used as media, for example.

In this embodiment, the mixing is performed with a ball mill using zirconia balls with a diameter of 1 mm by a dry method at 150 rpm for 1 hour. The mixing is performed in a dry room the dew point of which is higher than or equal to −100° C. and lower than or equal to −10° C.

### <Step S32>

Next, in Step S32 in FIG. 5A, the materials mixed in the above manner are collected, whereby a mixture 903 is obtained.

Note that this embodiment describes a method for adding the mixture of lithium fluoride and magnesium fluoride to lithium cobalt oxide with few impurities; however, one embodiment of the present invention is not limited thereto. A mixture obtained through heating after addition of a magnesium source, a fluorine source, and the like to the starting material of lithium cobalt oxide may be used instead of the mixture 903 in Step S32. In that case, there is no need to separate steps of Step S11 to Step S14 and steps of Step S21 to Step S23, which is simple and productive.

Alternatively, lithium cobalt oxide to which magnesium and fluorine are added in advance may be used. When lithium cobalt oxide to which magnesium and fluorine are added is used, the process can be simpler because the steps up to Step S32 can be omitted.

Alternatively, a magnesium source and a fluorine source may be further added to the lithium cobalt oxide to which magnesium and fluorine are added in advance.

### <Step S33>

Next, in Step S33, the mixture 903 is heated in an oxygen-containing atmosphere. The heating is preferably performed to prevent particles of the mixture 903 from adhering to one another.

The additive is preferably added to the entire surface of the particle not unevenly but uniformly. However, when particles of the mixture 903 adhere to one another during the heating, the additive might be unevenly added to part of the surface. A surface of the particle, which is preferably smooth and even, might become uneven due to adhered particles and have more defects such as a split and/or a crack. This is probably because the adhesion of the particles of the mixture 903 reduces the contact area with oxygen in the atmosphere and blocks a path through which the additives diffuse.

As the heating in Step S33, heating by a rotary kiln may be performed. Heating by a rotary kiln can be performed while stirring is performed in either case of a sequential rotary kiln or a batch-type rotary kiln. As the heating in Step S33, heating by a roller hearth kiln may be performed.

The heating temperature in Step S33 needs to be higher than or equal to the temperature at which a reaction between LiMO₂ and the additive element X source proceeds. Here, the temperature at which the reaction proceeds is a temperature at which interdiffusion between elements contained in LiMO₂ and the additive element X source occurs. Thus, the heating temperature can be lower than the melting temperatures of these materials in some cases. For example, in an oxide, solid-phase diffusion occurs at a temperature that is 0.757 times (Tamman temperature T_{d}) or more the melting temperature Tₘ. Accordingly, the heating temperature in Step S33 is higher than or equal to 500° C., for example.

Note that a temperature higher than or equal to the temperature at which at least part of the mixture 903 is melted is preferable because the reaction proceeds more easily. For example, in the case where LiF and MgF₂ are included as the additive element X source, the eutectic point of LiF and MgF₂ is around 742° C., and the heating temperature in Step S33 is preferably higher than or equal to 742° C.

The mixture 903 obtained by mixing such that LiCoO₂:LiF:MgF₂=100:0.33:1 (molar ratio) exhibits an endothermic peak at around 830° C. in differential scanning calorimetry measurement (DSC measurement). Thus, the heating temperature is further preferably higher than or equal to 830° C.

A higher heating temperature is preferable because it facilitates the reaction, shortens the heating time, and enables high productivity.

Note that the heating temperature needs to be lower than a decomposition temperature of LiMO₂ (1130° C. in the case of LiCoO₂). At around the decomposition temperature, a slight amount of LiMO₂ might be decomposed. Thus, the heating temperature in Step S33 is preferably lower than 1130° C., further preferably lower than or equal to 1000° C., further preferably lower than or equal to 950° C., further preferably lower than or equal to 900° C.

Therefore, the temperature of the heating in Step S33 is preferably higher than or equal to 500° C. and lower than 1130° C., further preferably higher than or equal to 500° C. and lower than or equal to 1000° C., still further preferably higher than or equal to 500° C. and lower than or equal to 950° C., yet still further preferably higher than or equal to 500° C. and lower than or equal to 900° C. Furthermore, the temperature is preferably higher than or equal to 742° C. and lower than 1130° C., further preferably higher than or equal to 742° C. and lower than or equal to 1000° C., still further preferably higher than or equal to 742° C. and lower than or equal to 950° C., yet still further preferably higher than or equal to 742° C. and lower than or equal to 900° C. Furthermore, the temperature is preferably higher than or equal to 830° C. and lower than 1130° C., further preferably higher than or equal to 830° C. and lower than or equal to 1000° C., still further preferably higher than or equal to 830° C. and lower than or equal to 950° C., yet still further preferably higher than or equal to 830° C. and lower than or equal to 900° C.

In addition, at the time of heating the mixture 903, the partial pressure of fluorine or a fluoride in the atmosphere is preferably controlled to be within an appropriate range.

In the formation method described in this embodiment, some of the materials, e.g., LiF as the fluorine source, function as a flux in some cases. Owing to this function, the heating temperature can be lower than or equal to the decomposition temperature of LiMO₂, e.g., a temperature higher than or equal to 742° C. and lower than or equal to 950° C., which allows distribution of the additive such as magnesium in the vicinity of the surface and formation of the positive electrode active material having favorable characteristics.

However, since LiF in a gas phase has a specific gravity less than that of oxygen, heating might volatilize LiF and in that case, LiF in the mixture 903 decreases. As a result, the function of a flux deteriorates. Thus, heating needs to be performed while volatilization of LiF is inhibited. Note that even when LiF is not used as the fluorine source or the like, there is a possibility in that Li and F at a surface of LiMO₂ react with each other to generate LiF and volatilize. Therefore, the volatilization needs to be inhibited also when a fluoride having a higher melting point than LiF is used.

In view of this, the mixture 903 is preferably heated in an atmosphere containing LiF, i.e., the mixture 903 is preferably heated in a state where the partial pressure of LiF in a heating furnace is high. Such heating can inhibit volatilization of LiF in the mixture 903.

In the case of using a rotary kiln for the heating, the flow rate of an oxygen-containing atmosphere in the kiln is preferably controlled while the mixture 903 is heated. For example, the flow rate of an oxygen-containing atmosphere is preferably set low, or no flowing of an oxygen gas is preferably performed after an atmosphere is purged first and an oxygen gas is introduced into the kiln.

In the case of using a roller hearth kiln for the heating, the mixture 903 can be heated in an atmosphere containing LiF with the container containing the mixture 903 covered with a lid, for example.

The heating is preferably performed for an appropriate time. The appropriate heating time is changed depending on conditions, such as the heating temperature, and the particle size and composition of LiMO₂ in Step S14. In the case where the particle size is small, the annealing is preferably performed at a lower temperature or for a shorter time than annealing in the case where the particle size is large, in some cases.

When the average particle diameter (D50) of the particles of the composite oxide in Step S14 in FIG. 5A is approximately 12 μm, for example, the heating temperature is preferably higher than or equal to 600° C. and lower than or equal to 950° C., for example. The heating time is preferably longer than or equal to 3 hours, further preferably longer than or equal to 10 hours, still further preferably longer than or equal to 60 hours, for example.

On the other hand, when the average particle diameter (D50) of the particles of the composite oxide in Step S14 is approximately 5 μm, the heating temperature is preferably higher than or equal to 600° C. and lower than or equal to 950° C., for example. The heating time is preferably longer than or equal to 1 hour and shorter than or equal to 10 hours, further preferably approximately 2 hours, for example. The temperature decreasing time after the heating is, for example, preferably longer than or equal to 10 hours and shorter than or equal to 50 hours.

### <Step S34>

Then, the heated materials are collected, whereby the positive electrode active material 100 is formed. Here, the collected particles are preferably made to pass through a sieve. Through the above steps, the positive electrode active material 100 of one embodiment of the present invention can be formed (Step S34).

### [Formation Method 3 of Positive Electrode Active Material]

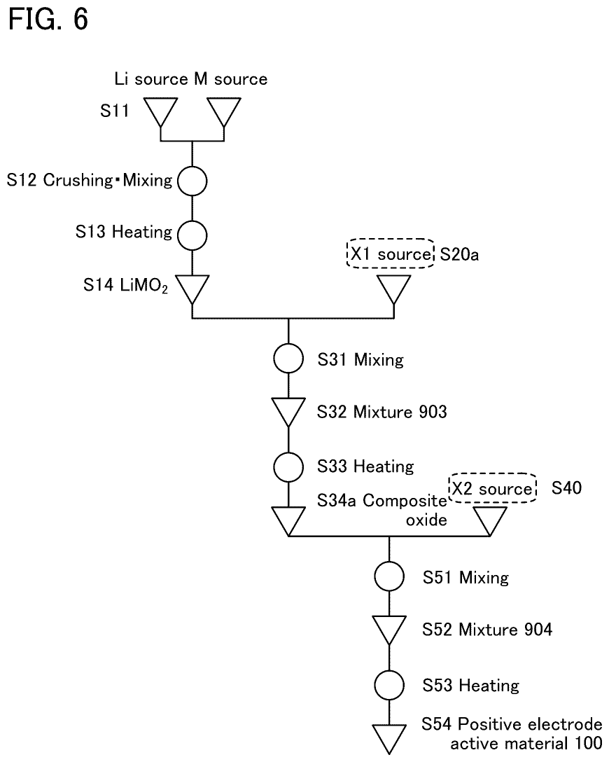

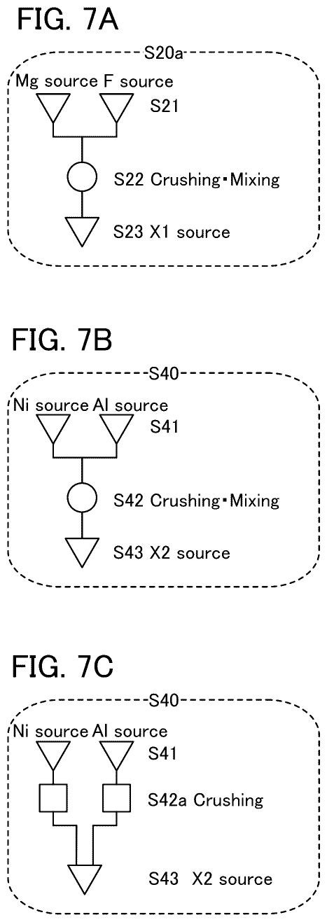

Next, another example of a method for forming a positive electrode active material of one embodiment of the present invention will be described with reference to FIG. 6, FIG. 7A, FIG. 7B, and FIG. 7C.

In FIG. 6, Steps S11 to S14 are performed as in FIG. 4A to prepare a composite oxide containing lithium, a transition metal, and oxygen (LiMO₂).

Note that a pre-synthesized composite oxide containing lithium, the transition metal, and oxygen may be used in Step S14. In that case, Step S11 to Step S13 can be omitted.

Note that a step of performing heating may be provided between Step S14 and the following Step S20 as described with reference to FIG. 5. For the heating, the conditions that are the same as the atmosphere and temperature for Step S33 described later are used and the treatment time is shorter than that for Step S33.

### <Step S20a>

In Step S20a in FIG. 6, an additive element X1 source is prepared. For the additive element X1 source, any of the above-described additive elements X can be selected to be used. For example, one or more selected from magnesium, fluorine, and calcium can be suitably used as the additive element X1. In this embodiment, an example in which magnesium and fluorine are used as the additive elements X1 is shown with reference to FIG. 7A. Step S21 and Step S22 included in Step S20a shown in FIG. 7A can be performed in a manner similar to that in Step S21 and Step S22 shown in FIG. 5B. For the addition of the additive element X1, a solid phase method, a liquid phase method such as a sol-gel method, a sputtering method, an evaporation method, a CVD (chemical vapor deposition) method, a PLD (pulsed laser deposition) method, and the like can be used.

Step S23 shown in FIG. 7A is a step in which the materials ground and mixed in Step S22 shown in FIG. 7A are collected to obtain the additive element X1 source.

Steps S31 to S33 shown in FIG. 6 can be performed in a manner similar to that of Steps S31 to S33 shown in FIG. 5.

### <Step S34a>

Next, the material heated in Step S33 is collected to form a composite oxide.

### <Step S40>

Then, in Step S40 in FIG. 6, an additive element X2 source is prepared. For the additive element X2 source, any of the above-described additive elements X can be selected to be used. For example, one or more selected from nickel, titanium, boron, zirconium, and aluminum can be suitably used as the additive element X2. In this embodiment, FIG. 7B shows an example of using nickel and aluminum as the additive elements X2. Step S41 and Step S42 included in Step S40 shown in FIG. 7B can be performed in a manner similar to that in Step S21 and Step S22 shown in FIG. 5B. For the addition of the additive element X2, a solid phase method, a liquid phase method such as a sol-gel method, a sputtering method, an evaporation method, a CVD (chemical vapor deposition) method, a PLD (pulsed laser deposition) method, and the like can be used.

Step S43 shown in FIG. 7B is a step in which the materials ground and mixed in Step S42 shown in FIG. 7B are collected to obtain the additive element X2 source.

Step S40 shown in FIG. 7C is a modification example of Step S40 shown in FIG. 7B. In FIG. 7C, a nickel source and an aluminum source are prepared (Step S41) and subjected to crushing (Step S42a) independently, whereby a plurality of additive element X2 sources are prepared (Step S43).

In the case of employing the sol-gel method for addition of the additive element X2, a solvent used for the sol-gel method is prepared as well as the additive element X2 source. For the sol-gel method, a metal alkoxide can be used as the metal source, for example, and alcohol can be used as the solvent, for example. In the case of performing addition of aluminum, aluminum isopropoxide can be used as the metal source and isopropanol (2-propanol) can be used as the solvent, for example. In the case of performing addition of zirconium, zirconium(IV) tetrapropoxide can be used as the metal source and isopropanol can be used as the solvent, for example.

### <Step S51 to Step S53>

Next, Step S51 in FIG. 6 is a step of mixing the composite oxide formed in Step S34a and the additive element X2 source formed in Step S40. Note that Step S51 in FIG. 6 can be performed in a manner similar to that in Step S31 shown in FIG. 5A. In addition, Step S52 in FIG. 6 can be performed in a manner similar to that in Step S32 shown in FIG. 5A. Note that a material formed in Step S52 in FIG. 6 is a mixture 904. The mixture 904 is a material containing, in addition to the material of the mixture 903, the additive element X2 added in Step S40. Step S53 in FIG. 6 can be performed in a manner similar to that in Step S33 shown in FIG. 5A.

### <Step S54>

Then, the heated materials are collected, whereby the positive electrode active material 100 is formed. Here, the collected particles are preferably made to pass through a sieve. Through the above steps, the positive electrode active material 100 of one embodiment of the present invention can be formed (Step S54).

When the step of introducing the transition metal, the step of introducing the additive element X1, and the step of introducing the additive element X2 are separately performed as shown in FIG. 6 and FIG. 7A to FIG. 7C, the element concentration profiles in the depth direction can be made different from each other in some cases. For example, the concentration of an additive can be made higher in the region in the vicinity of the surface than in the inner portion of the particle. Furthermore, with the number of atoms of the transition metal as a reference, the ratio of the number of atoms of the additive element with respect to the reference can be higher in the vicinity of the surface than in the inner portion.

The formation method in which a high-purity material is used for the transition metal source used in synthesis; a process which hardly allows entry of impurities in the synthesis is employed; entry of impurities in the synthesis is thoroughly prevented; and desired additive elements (the additive element X, the additive element X1, or the additive element X2) are controlled to be introduced into the positive electrode active material can provide a positive electrode active material in which a region with a low impurity concentration and a region where the additive elements are introduced are controlled. In addition, the positive electrode active material having high crystallinity can be obtained. Furthermore, the positive electrode active material obtained by the method for forming a positive electrode active material, which is one embodiment of the present invention, can increase the capacity of a secondary battery and/or increase the reliability of the secondary battery.

### [Positive Electrode Active Material 2]

The positive electrode active material of one embodiment of the present invention is not limited to the materials described above. A mixture of the above-described material and another material may be used as the positive electrode active material of one embodiment of the present invention.

As the positive electrode active material, a composite oxide with a spinel crystal structure can be used, for example. Alternatively, a polyanionic material can be used as the positive electrode active material, for example. Examples of the polyanionic material include a material with an olivine crystal structure and a material with a NASICON structure. Alternatively, a material containing sulfur can be used as the positive electrode active material, for example.

As the material with a spinel crystal structure, for example, a composite oxide represented by LiM₂O₄ can be used. It is preferable to contain Mn as the metal M. For example, LiMn₂O₄ can be used. It is preferable to contain Ni in addition to Mn as the metal M because the discharge voltage and the energy density of the secondary battery are increased in some cases. It is preferable to add a small amount of lithium nickel oxide (LiNiO₂ or LiNi₁₋ₓMₓO₂ (M=Co, Al, or the like)) to a lithium-containing material with a spinel crystal structure which contains manganese, such as LiMn₂O₄, because the characteristics of the secondary battery can be improved.

As a polyanionic material, for example, a composite oxide containing oxygen, the metal A, the metal M, and the element X can be used. The metal A is one or more of Li, Na, and Mg; the metal M is one or more of Fe, Mn, Co, Ni, Ti, V, and Nb; and the element X is one or more of S, P, Mo, W, As, and Si.

As the material with an olivine crystal structure, for example, a composite material (the general formula LiMPO₄ (M is one or more of Fe(II), Mn(II), Co(II), and Ni(II)) can be used. Typical examples of the general formula LiMPO₄ include lithium compounds such as LiFePO₄, LiNiPO₄, LiCoPO₄, LiMnPO₄, LiFeₐNi_{b}PO₄, LiFeₐCo_{b}PO₄, LiFeₐMn_{b}PO₄, LiNiₐCo_{b}PO₄, LiNiₐMn_{b}PO₄ (a+b≤1, 0<a<1, and 0<b<1), LiFe_{c}Ni_{d}CoₑPO₄, LiFe_{c}Ni_{d}MnₑPO₄, LiNi_{c}Co_{d}MnₑPO₄ (c+d+e≤1, 0<c<1, 0<d<1, and 0<e<1), and LiFe_{f}Ni_{g}CoₕMnᵢPO₄ (f+g+h+i≤1, 0<f<1, 0<g<1, 0<h<1, and 0<i<1).

Alternatively, a composite material such as a general formula Li₍₂₋ⱼ₎MSiO₄ (M is one or more of Fe(II), Mn(II), Co(II), and Ni(II); 0≤j≤2) can be used. Typical examples of the general formula Li₍₂₋ⱼ₎MSiO₄ include lithium compounds such as Li₍₂₋ⱼ₎FeSiO₄, Li₍₂₋ⱼ₎CoSiO₄, Li₍₂₋ⱼ₎MnSiO₄, Li₍₂₋ⱼ₎FeₖNiₗSiO₄, Li₍₂₋ⱼ₎FeₖCoₗSiO₄, Li₍₂₋ⱼ₎FeₖMnₗSiO₄, Li₍₂₋ⱼ₎NiₖCoₗSiO₄, Li₍₂₋ⱼ₎NiₖMnₗSiO₄ (k+l≤1, 0<k<1, and 0<l<1), Li₍₂₋ⱼ₎FeₘNiₙCo_{q}SiO₄, Li₍₂₋ⱼ₎FeₘNiₙMn_{q}SiO₄, Li₍₂₋ⱼ₎NiₘCoₙMn_{q}SiO₄ (m+n+q≤1, 0<m<1, 0<n<1, and 0<q<1), and Li₍₂₋ⱼ₎FeᵣNiₛCoₜMnᵤSiO₄ (r+s+t+u≤1, 0<r<1, 0<s<1, 0<t<1, and 0<u<1).

Still alternatively, a NASICON compound represented by a general formula AₓM₂(XO₄)₃ (A=Li, Na, or Mg, M=Fe, Mn, Ti, V, or Nb, X=S, P, Mo, W, As, or Si) can be used. Examples of the NASICON compound include Fe₂(MnO₄)₃, Fe₂(SO₄)₃, and Li₃Fe₂(PO₄)₃. Further alternatively, a compound represented by a general formula Li₂MPO₄F, Li₂MP₂O₇, or Li₅MO₄ (M=Fe or Mn) can be used as the positive electrode active material.

Further alternatively, a perovskite fluoride such as NaFeF₃ and FeF₃, a metal chalcogenide (a sulfide, a selenide, or a telluride) such as TiS₂ and MoS₂, an oxide with an inverse spinel crystal structure such as LiMVO₄, a vanadium oxide (V₂O₅, V₆O₁₃, LiV₃O₈, or the like), a manganese oxide, an organic sulfur compound, or the like may be used as the positive electrode active material.

Alternatively, a borate-based material represented by a general formula LiMBO₃ (M is Fe(II), Mn(II), or Co(II)) may be used as the positive electrode active material.

As a material containing sodium, for example, an oxide containing sodium such as NaFeO₂, Na_{2/3}[Fe_{1/2}Mn_{1/2}]O₂, Na_{2/3}[Ni_{1/3}Mn_{2/3}]O₂, Na₂Fe₂(SO₄)₃, Na₃V₂(PO₄)₃, Na₂FePO₄F, NaVPO₄F, NaMPO₄ (M is Fe(II), Mn(II), Co(II), or Ni(II)), Na₂FePO₄F, or Na₄Co₃(PO₄)₂P₂O₇ may be used as the positive electrode active material.

As the positive electrode active material, a lithium-containing metal sulfide may be used. Examples of the lithium-containing metal sulfide are Li₂TiS₃ and Li₃NbS₄.

### [Electrolyte]

The secondary battery of one embodiment of the present invention preferably includes an electrolyte solution. The electrolyte solution included in the secondary battery of one embodiment of the present invention preferably contains an ionic liquid and a salt containing a metal serving as a carrier ion.

In the case where the metal serving as a carrier ion is lithium, as the salt containing the metal serving as a carrier ion, one of lithium salts such as LiN(FSO₂)₂, LiN(CF₃SO₂)₂, LiN(C₄F₉SO₂) (CF₃SO₂), LiN(C₂F₅SO₂)₂, LiC(FSO₂)₃, LiC(CF₃SO₂)₃, LiC(C₂F₅SO₂)₃, LiCF₃SO₃, LiC₄F₉SO₃, LiAsF₆, LiBF₄, LiAlCl₄, LiSCN, LiBr, LiI, Li₂SO₄, Li₂B₁₀Cl₁₀, Li₂B₁₂Cl₁₂, LiPF₆, and LiClO₄ can be used, or two or more of them can be used in an appropriate combination in an appropriate ratio.

In particular, a metal salt of a fluorosulfonate anion and a metal salt of a fluoroalkylsulfonate anion are preferable: among them, a metal salt of an amide-based anion represented by (CₙF₂ₙ₊₁SO₂)₂N⁻ (n is greater than or equal to 0 and less than or equal to 3) is preferable because of its high stability at high temperatures and high resistance to oxidation reduction.

An ionic liquid contains a cation and an anion, specifically, an organic cation and an anion. Examples of the organic cation used for the electrolyte solution include aromatic cations such as an imidazolium cation and a pyridinium cation, and aliphatic onium cations such as a quaternary ammonium cation, a tertiary sulfonium cation, and a quaternary phosphonium cation. Examples of the anion used for the electrolyte solution include a monovalent amide-based anion, a monovalent methide-based anion, a fluorosulfonate anion, a perfluoroalkylsulfonate anion, a tetrafluoroborate anion, a perfluoroalkylborate anion, a hexafluorophosphate anion, and a perfluoroalkylphosphate anion.

The electrolyte solution may contain, in addition to an ionic liquid, an aprotic solvent. For example, one of ethylene carbonate (EC), propylene carbonate (PC), butylene carbonate, chloroethylene carbonate, vinylene carbonate, γ-butyrolactone, γ-valerolactone, dimethyl carbonate (DMC), diethyl carbonate (DEC), ethyl methyl carbonate (EMC), methyl formate, methyl acetate, ethyl acetate, methyl propionate, ethyl propionate, propyl propionate, methyl butyrate, 1,3-dioxane, 1,4-dioxane, dimethoxyethane (DME), dimethyl sulfoxide, diethyl ether, methyl diglyme, acetonitrile, benzonitrile, tetrahydrofuran, sulfolane, and sultone may be contained, or two or more of these solvents may be contained in an appropriate combination in an appropriate ratio.

Furthermore, an additive such as vinylene carbonate (VC); propane sultone (PS); tert-butylbenzene (TBB); fluoroethylene carbonate (FEC); lithium bis(oxalate)borate (LiBOB); a dinitrile compound such as succinonitrile or adiponitrile; fluorobenzene; cyclohexylbenzene; or biphenyl may be added to the electrolyte solution. The concentration of the material to be added in the whole solvent is, for example, higher than or equal to 0.1 wt % and lower than or equal to 5 wt %.

As an ionic liquid containing imidazolium cations, an ionic liquid represented by General Formula (G1) below can be used, for example. In General Formula (G1), R¹ represents an alkyl group having 1 to 6 carbon atoms or a substituted or unsubstituted aryl group having 6 to 13 carbon atoms and preferably represents an alkyl group having 1 to 4 carbon atoms, R² to R⁴ each independently represent a hydrogen atom or an alkyl group having 1 to 6 carbon atoms or a substituted or unsubstituted aryl group having 6 to 13 carbon atoms and preferably represent an alkyl group having 1 to 4 carbon atoms, and R⁵ represents an alkyl group or a main chain composed of two or more selected from C, O, Si, N, S, and P atoms. A substituent may be introduced into the main chain represented by R⁵. Examples of the substituent to be introduced include an alkyl group and an alkoxy group. The main chain represented by R⁵ may have a carboxy group. The main chain represented by R⁵ may have a carbonyl group.

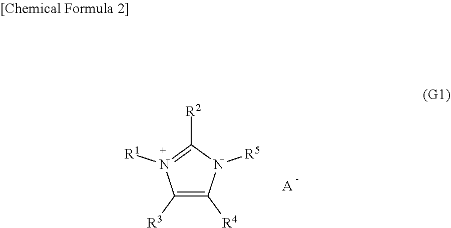

As an ionic liquid containing pyridinium cations, an ionic liquid represented by General Formula (G2) below may be used, for example. In General Formula (G2), R⁶ represents an alkyl group or a main chain composed of two or more selected from C, O, Si, N, S, and P atoms, and R⁷ to R¹¹ each independently represent a hydrogen atom or an alkyl group having 1 to 4 carbon atoms. A substituent may be introduced into the main chain represented by R⁶. Examples of the substituent to be introduced include an alkyl group and an alkoxy group.

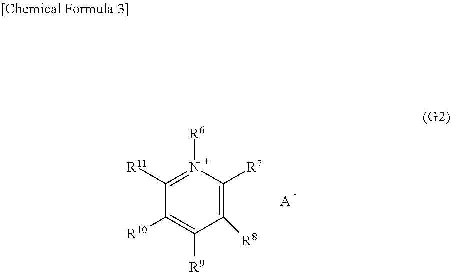

As an ionic liquid containing quaternary ammonium cations, an ionic liquid represented by General Formula (G3), (G4), (G5), or (G6) below can be used, for example.

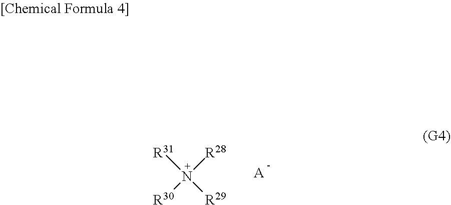

In General Formula (G3), R²⁸ to R³¹ each independently represent an alkyl group having 1 to 20 carbon atoms, a methoxy group, a methoxymethyl group, a methoxyethyl group, or a hydrogen atom.

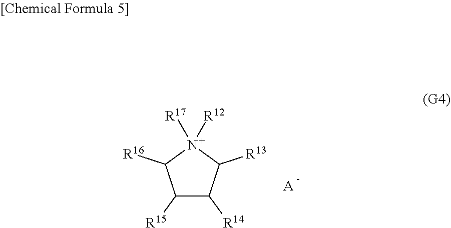

In General Formula (G4), R¹² to R¹⁷ each independently represent an alkyl group having 1 to 20 carbon atoms, a methoxy group, a methoxymethyl group, a methoxyethyl group, or a hydrogen atom.

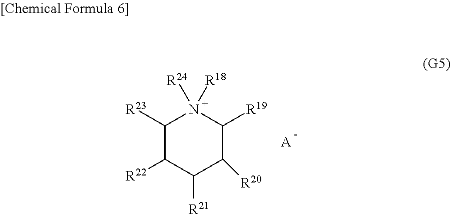

In General Formula (G5), R¹⁸ to R²⁴ each independently represent an alkyl group having 1 to 20 carbon atoms, a methoxy group, a methoxymethyl group, a methoxyethyl group, or a hydrogen atom.

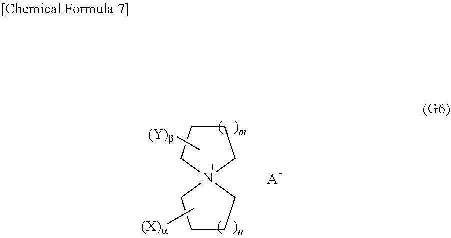

In General Formula (G6), n and m are greater than or equal to 1 and less than or equal to 3. Assume that α is greater than or equal to 0 and less than or equal to 6. When n is 1, α is greater than or equal to 0 and less than or equal to 4. When n is 2, α is greater than or equal to 0 and less than or equal to 5. When n is 3, α is greater than or equal to 0 and less than or equal to 6. Assume that β is greater than or equal to 0 and less than or equal to 6. When m is 1, β is greater than or equal to 0 and less than or equal to 4. When m is 2, β is greater than or equal to 0 and less than or equal to 5. When m is 3, β is greater than or equal to 0 and less than or equal to 6. Note that “α or β is 0” means “unsubstituted”. The case where both α and β are 0 is excluded. X or Y represents a substituent such as a straight-chain or side-chain alkyl group having 1 to 4 carbon atoms, a straight-chain or side-chain alkoxy group having 1 to 4 carbon atoms, or a straight-chain or side-chain alkoxyalkyl group having 1 to 4 carbon atoms.

As an ionic liquid containing tertiary sulfonium cations, an ionic liquid represented by General Formula (G7) below can be used, for example. In General Formula (G7), R²⁵ to R²⁷ each independently represent a hydrogen atom, an alkyl group having 1 to 4 carbon atoms, or a phenyl group. Alternatively, as R²⁵ to R²⁷, a main chain composed of two or more selected from C, O, Si, N, S, and P atoms may be used.

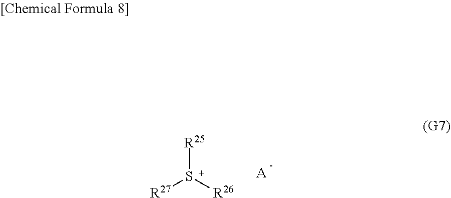

As an ionic liquid containing quaternary phosphonium cations, an ionic liquid represented by General Formula (G8) below can be used, for example. In General Formula (G8), R³² to R³⁵ each independently represent a hydrogen atom, an alkyl group having 1 to 4 carbon atoms, or a phenyl group. Alternatively, as R³² to R³⁵, a main chain composed of two or more selected from C, O, Si, N, S, and P atoms may be used.

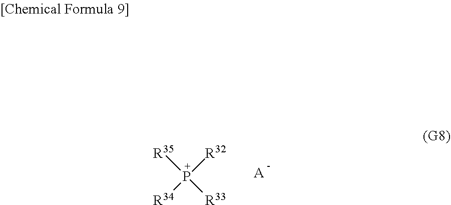

As A⁻ shown in General Formulae (G1) to (G8), one or more of a monovalent amide-based anion, a monovalent methide-based anion, a fluorosulfonate anion, a perfluoroalkylsulfonate anion, a tetrafluoroborate anion, a perfluoroalkylborate anion, a hexafluorophosphate anion, and a perfluoroalkylphosphate anion can be used.

As a monovalent amide-based anion, (CₙF₂ₙ₊₁SO₂)₂N⁻ (n=0 to 3) can be used, and as a monovalent cyclic amide-based anion, (CF₂SO₂)₂N⁻ or the like can be used. As a monovalent methide-based anion, (CₙF₂ₙ₊₁SO₂)₃C⁻ (n=0 to 3) can be used, and as a monovalent cyclic methide-based anion, (CF₂SO₂)₂C⁻ (CF₃SO₂) or the like can be used. As a fluoroalkyl sulfonic acid anion, (CₘF₂ₘ₊₁SO₃)⁻ (m=0 to 4) or the like is given. As a fluoroalkylborate anion, {BFₙ(CₘHₖF₂ₘ₊₁₋ₖ)₄₋ₙ}⁻ (n=0 to 3, m=1 to 4, and k=0 to 2m) or the like is given. As a fluoroalkylphosphate anion, {PFₙ(CₘHₖF₂ₘ₊₁₋ₖ)₆₋ₙ}⁻ (n=0 to 5, m=1 to 4, and k=0 to 2m) or the like is given.

As a monovalent amide-based anion, one or more of a bis(fluorosulfonyl)amide anion and a bis(trifluoromethanesulfonyl)amide anion can be used, for example.

An ionic liquid may contain one or more of a hexafluorophosphate anion and a tetrafluoroborate anion.

Hereinafter, an anion represented by (FSO₂)₂N⁻ is sometimes represented by an FSA anion, and an anion represented by (CF₃SO₂)₂N⁻ is sometimes represented by a TFSA anion.

Specific examples of the cation represented by General Formula (G1) above include Structural Formula (111) to Structural Formula (174).

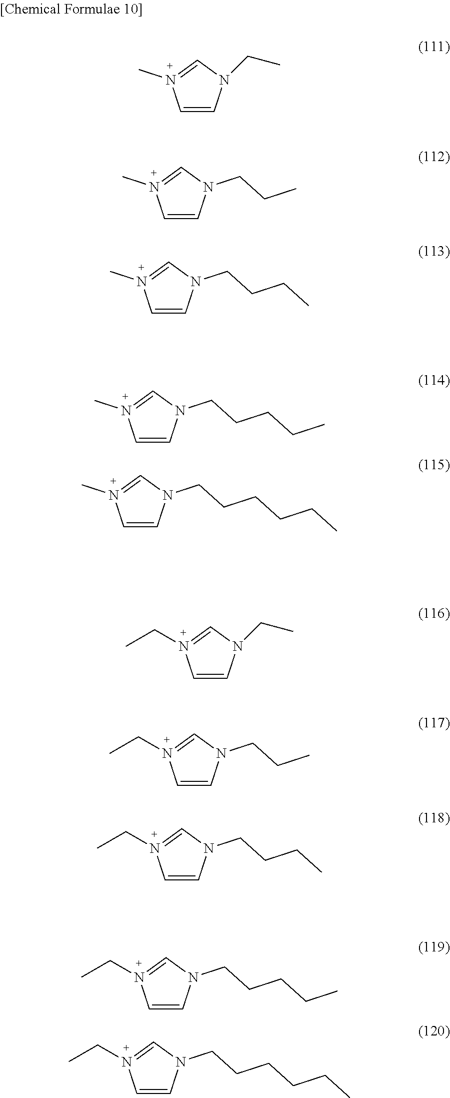

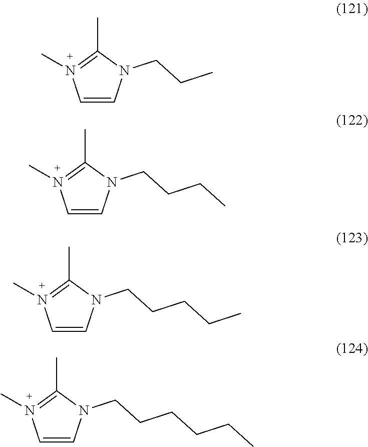

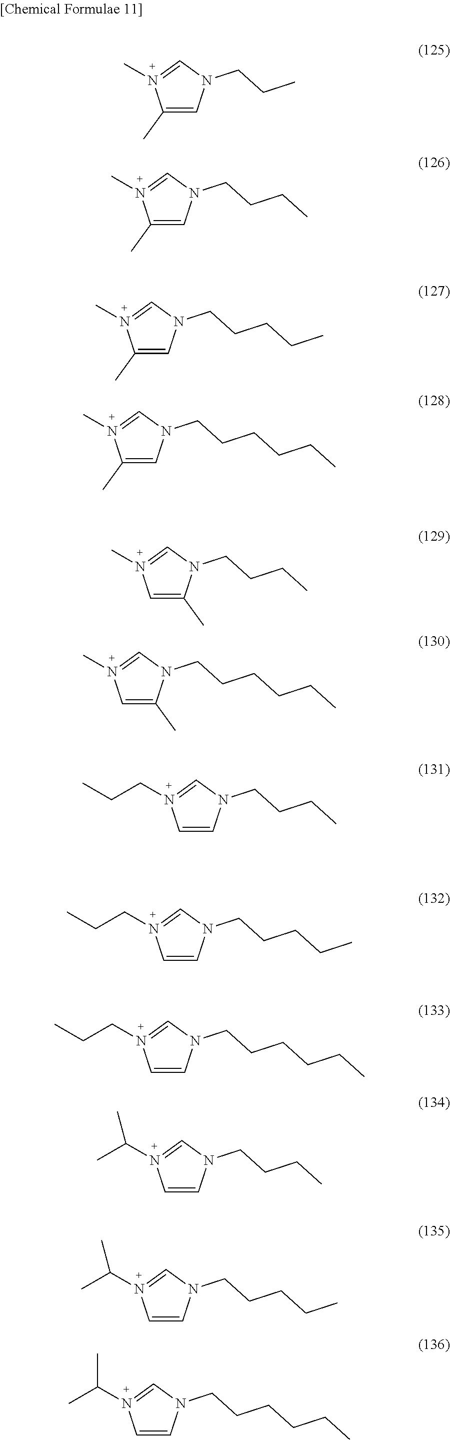

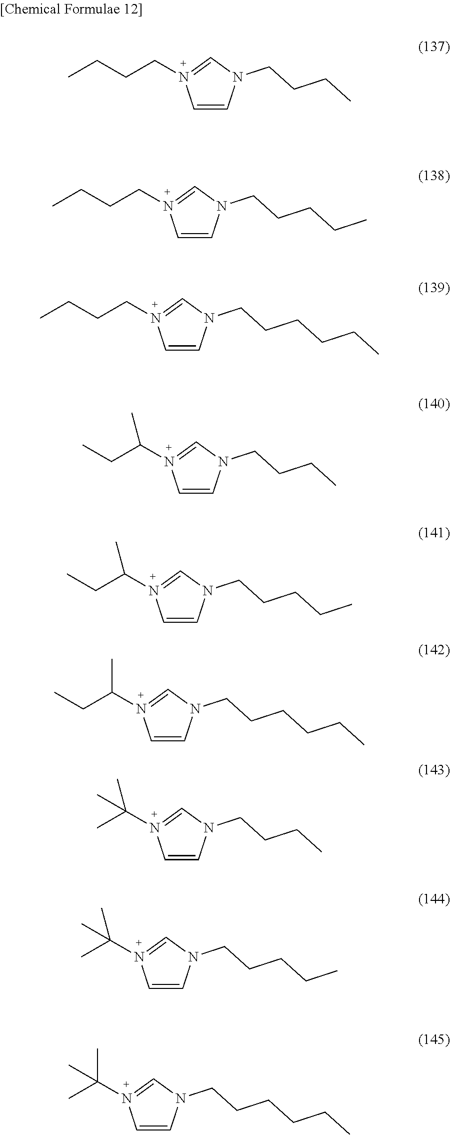

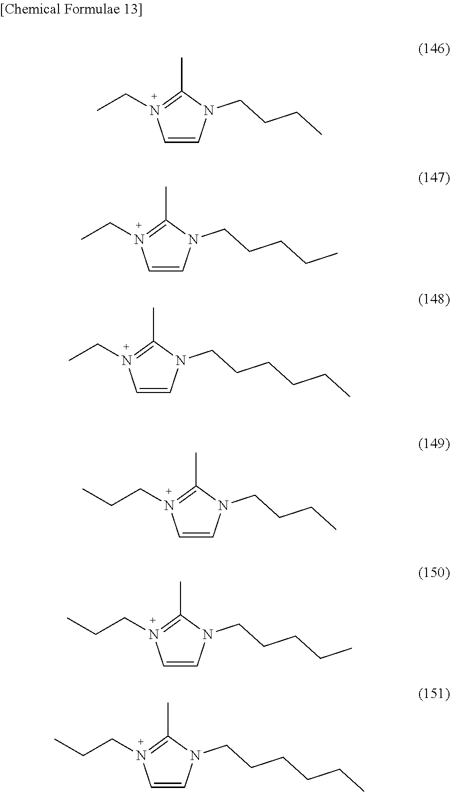

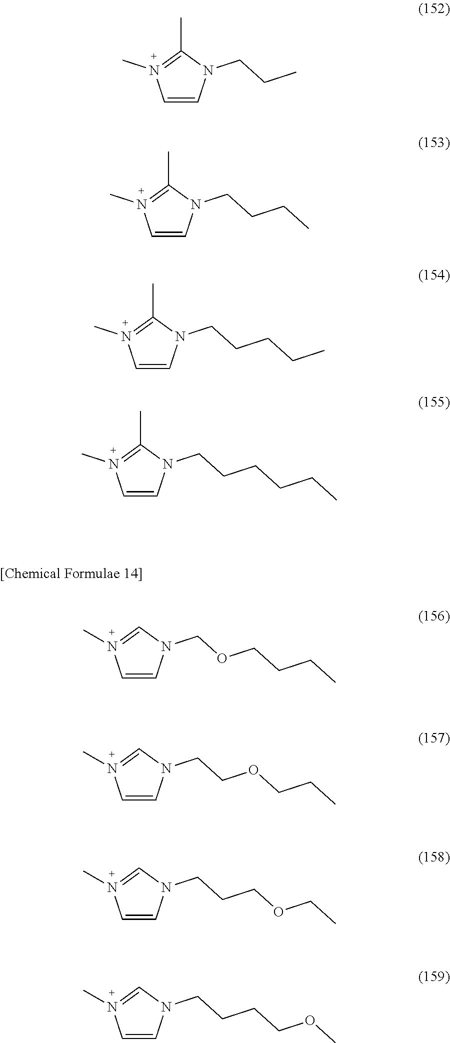

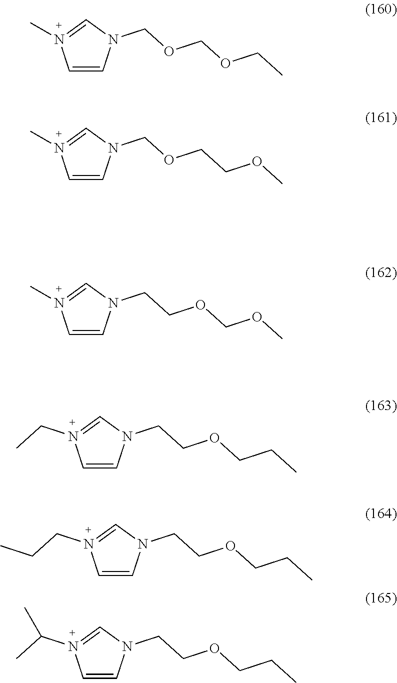

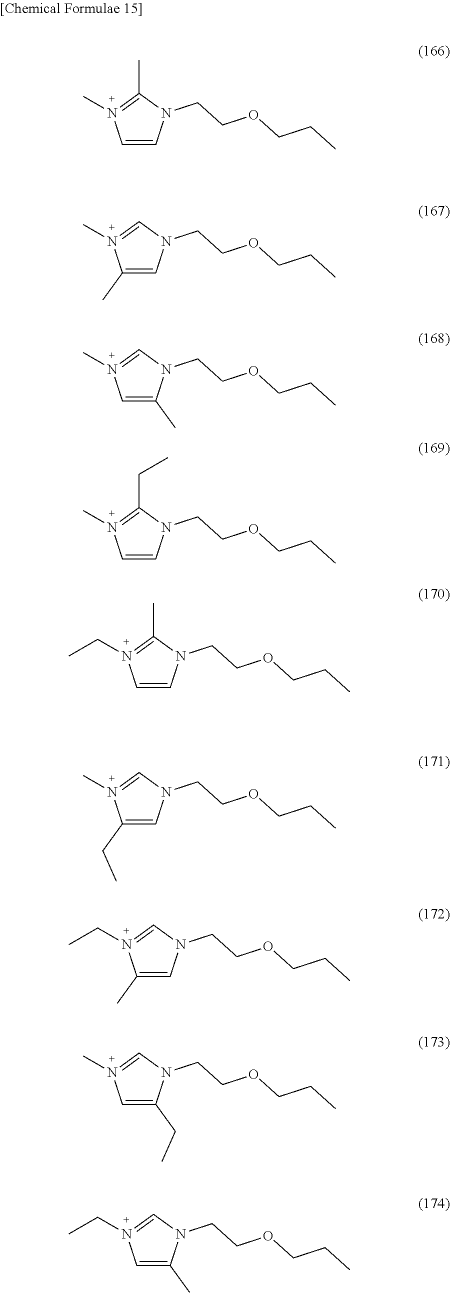

The ionic liquid shown in General Formula (G1) contains an imidazolium cation and an anion represented by A⁻. An ionic liquid containing an imidazolium cation has low viscosity and can be used in a wide temperature range. Moreover, an ionic liquid containing an imidazolium cation has high stability and a wide potential window and thus can be suitably used as an electrolyte of a secondary battery.

A mixture of the ionic liquid shown in General Formula (G1) and a salt such as a lithium salt can be used as an electrolyte of a secondary battery. The imidazolium cation shown in General Formula (G1) has high resistance to oxidation, high resistance to reduction, and a wide potential window and thus is suitable as a solvent used for an electrolyte. Here, the range of potentials in which the electrolysis of an electrolyte does not occur is referred to as a potential window. In particular, the secondary battery of one embodiment of the present invention includes a positive electrode active material that has excellent characteristics even at a high charge voltage and charge voltage can be increased. Thus, the use of an ionic liquid having a wide potential window and significantly high resistance to, in particular, oxidation can achieve an excellent secondary battery.

In particular, in General Formula (G1), when R¹ represents a methyl group, an ethyl group, or a propyl group; one of R², R³, and R⁴ represents a hydrogen atom or a methyl group and the other two represent hydrogen atoms; and either an anion represented by (FSO₂)₂N⁻ (an FSA anion) or an anion represented by (CF₃SO₂)₂N⁻ (a TFSA anion) or a mixture thereof is used as the anion A⁻, it is possible to achieve an electrolyte that has a wide potential window, has excellent resistance to oxidation, and can be used in a wide temperature range without being solidified even at a temperature at which viscosity lowers.

In particular, a metal salt of a fluorosulfonate anion and a metal salt of a fluoroalkylsulfonate anion are preferable as a salt used for an electrolyte: among them, a metal salt of an amide-based anion represented by (CₙF₂ₙ₊₁SO₂)₂N⁻ (n is greater than or equal to 0 and less than or equal to 3) is preferable because of its high stability at high temperatures and high resistance to oxidation reduction. In particular, by using either LiN(FSO₂)₂ or LiN(CF₃SO₂)₂ or a mixture thereof, a secondary battery that is highly stable and can operate in a wide temperature range can be achieved.

As examples of the cation in General Formula (G1) in which R¹ represents a methyl group, an ethyl group, or a propyl group and one of R², R³, and R⁴ represents a hydrogen atom or a methyl group and the other two represent hydrogen atoms, cations represented by Structural Formulae (111) to (124) above, Structural Formulae (131) to (136) above, Structural Formulae (146) to (155) above, and Structural Formulae (156) to (166) and (170) above are given. One selected from those cations is preferably used. Alternatively, a plurality of cations selected from those cations may be used in combination.

Furthermore, when the sum of carbon atoms and oxygen atoms contained in R¹ and R⁵ is less than or equal to 7 in General Formula (G1), the viscosity of an ionic liquid is lowered and a secondary battery with excellent output characteristics can be achieved. For example, among the above-described cations, a 1-butyl-3-propylimidazolium (BPI) cation represented by Structural Formula (131) above is preferably used.

For example, it is preferable to use a cation in General Formula (G1) in which R¹ represents a methyl group, R² represents a hydrogen atom, and the sum of carbon atoms and oxygen atoms contained in R⁵ is less than or equal to 6. An electrolyte of a secondary battery preferably contains one or more selected from the cations represented by Structural Formulae (111) to (115) and Structural Formulae (156) to (162) above. It is particularly preferable that an electrolyte of a secondary battery contain one or more selected from a 1-ethyl-3-methylimidazolium (EMI) cation represented by Structural Formula (111) above, a 1-butyl-3-methylimidazolium (BMI) cation represented by Structural Formula (113) above, a 1-hexyl-3-methylimidazolium (HMI) cation represented by Structural Formula (115) above, and a 1-methyl-3-(2-propoxyethyl)imidazolium (poEMI) cation represented by Structural Formula (157) above. In particular, an ionic liquid containing the EMI cation is suitable because of its low viscosity and extremely high stability.

By using a mixture of the EMI cation and the BMI cation, for example, an ionic liquid having low viscosity and high stability can be achieved. In the case where a mixture of the EMI cation and the BMI cation is used, for example, the EMI cation: the BMI cation is e:b (molar ratio) where e>b is satisfied; alternatively, e>2b may be satisfied.

A mixture of the ionic liquid shown in General Formula (G1) and one or more selected from ionic liquids shown in General Formulae (G2) to (G8) has low viscosity and can be used in a wide temperature range. Therefore, an ionic liquid having particularly high resistance to oxidation and extremely high stability can be achieved. In that case, for example, it is preferable that the volume of the ionic liquid shown in General Formula (G1) be larger than the volume of one or more selected from the ionic liquids shown in General Formulae (G2) to (G8), and it is further preferable that the volume of the ionic liquid shown in General Formula (G1) be larger than twice the volume of one or more selected from the ionic liquids shown in General Formulae (G2) to (G8).

Specific examples of the cation represented by General Formula (G2) above include Structural Formula (701) to Structural Formula (719).

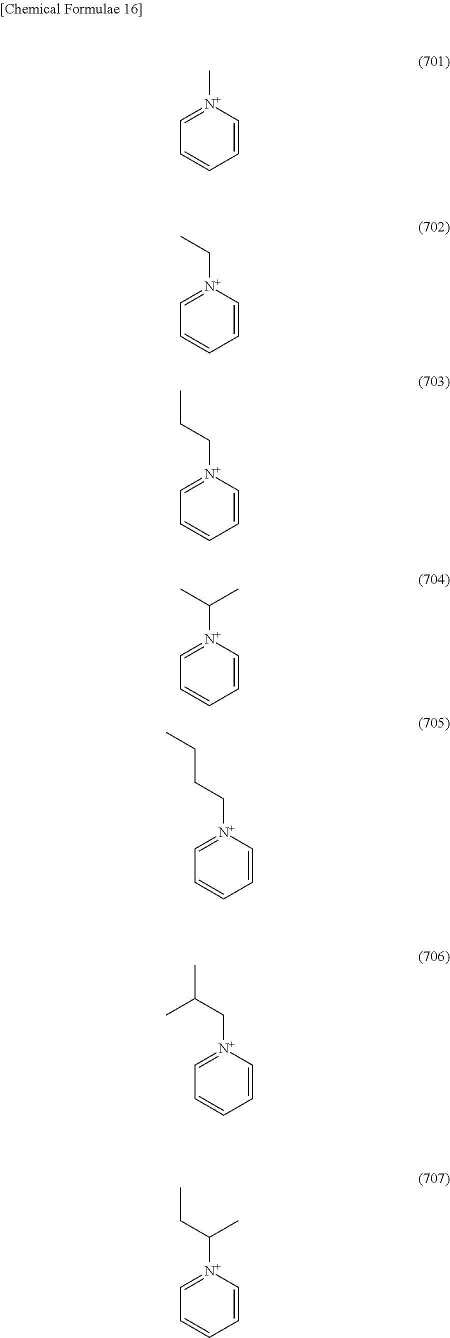

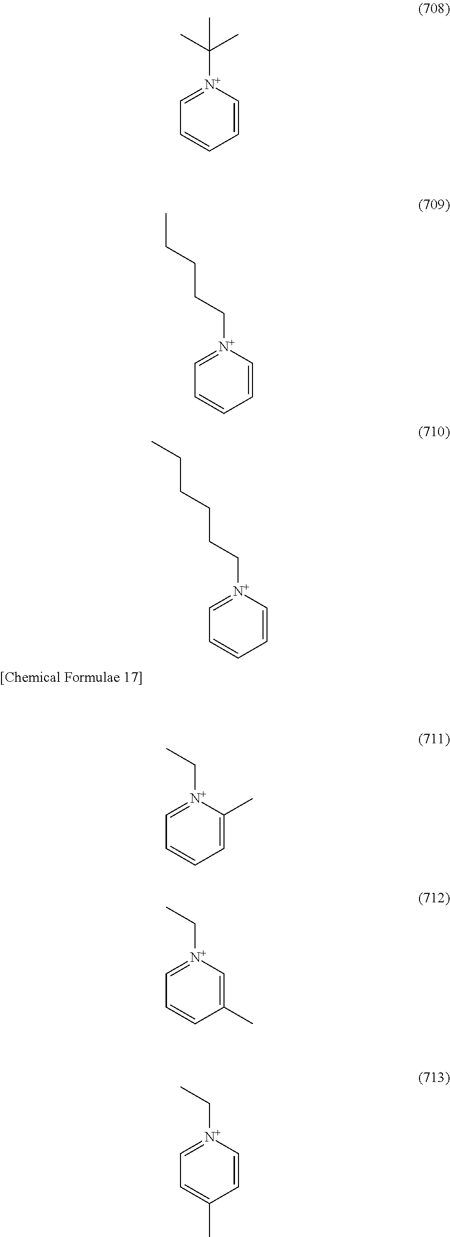

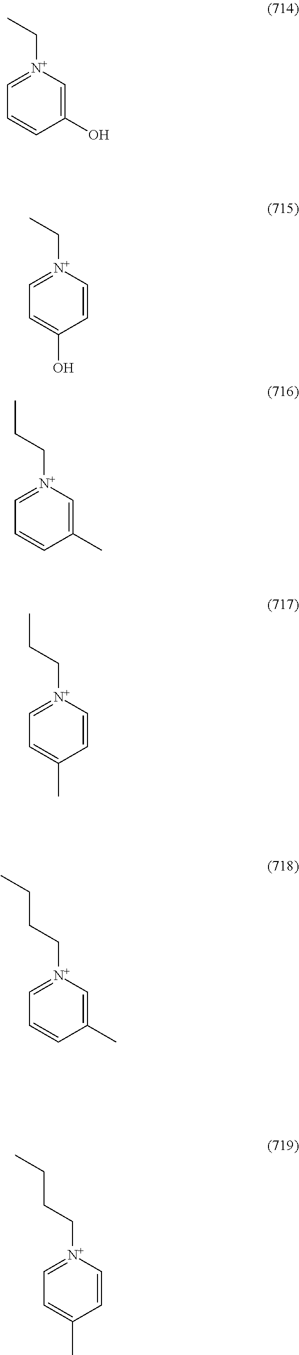

Specific examples of the cation represented by General Formula (G4) above include Structural Formula (501) to Structural Formula (520).

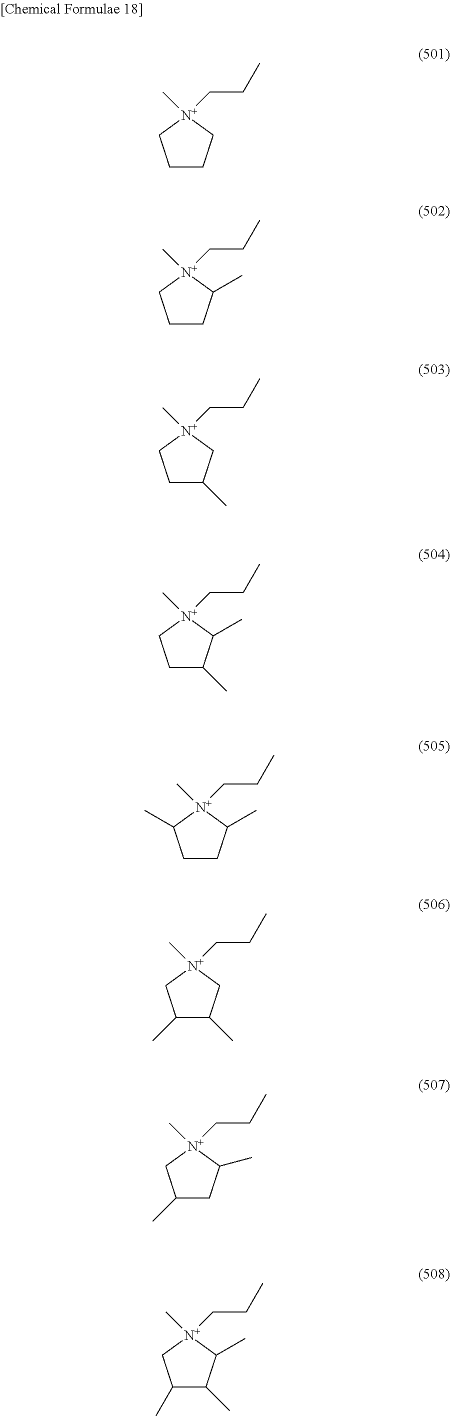

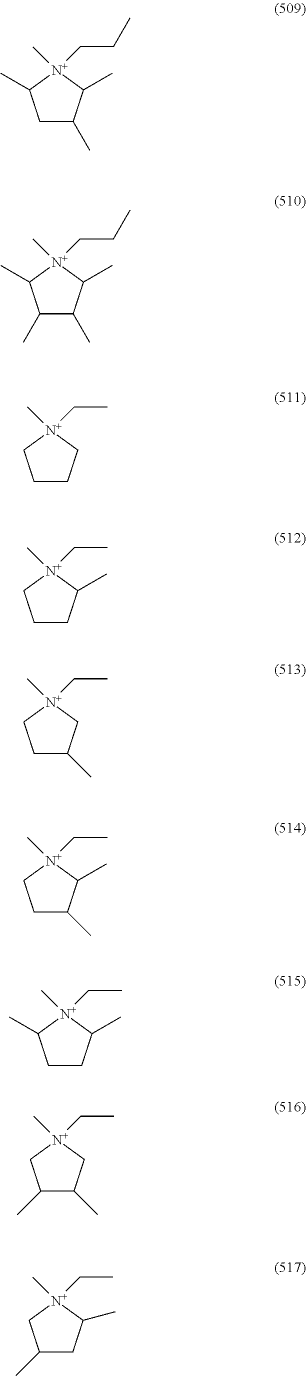

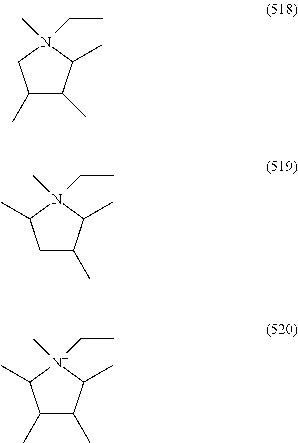

Specific examples of the cation represented by General Formula (G5) above include Structural Formula (601) to Structural Formula (630).

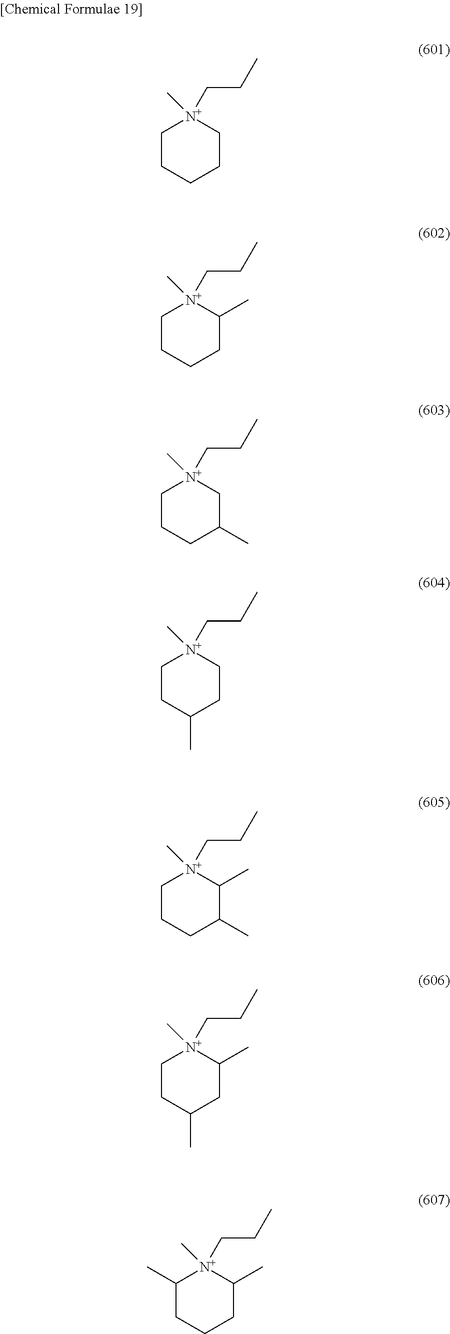

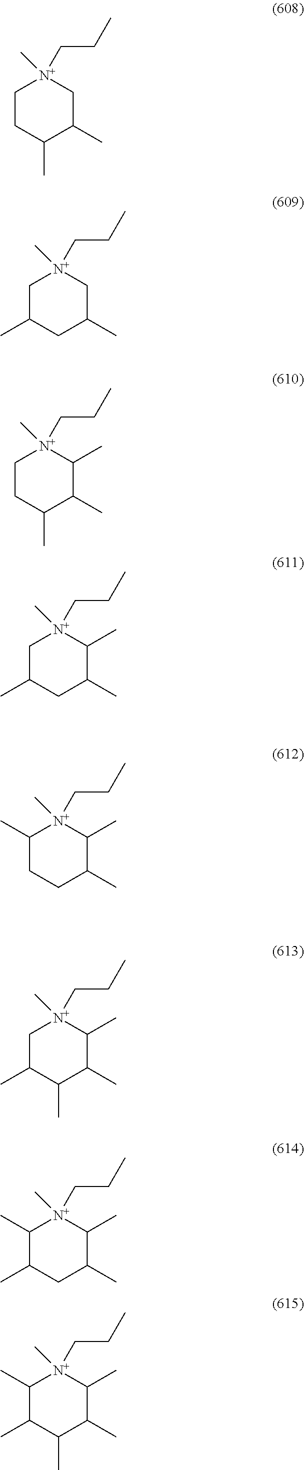

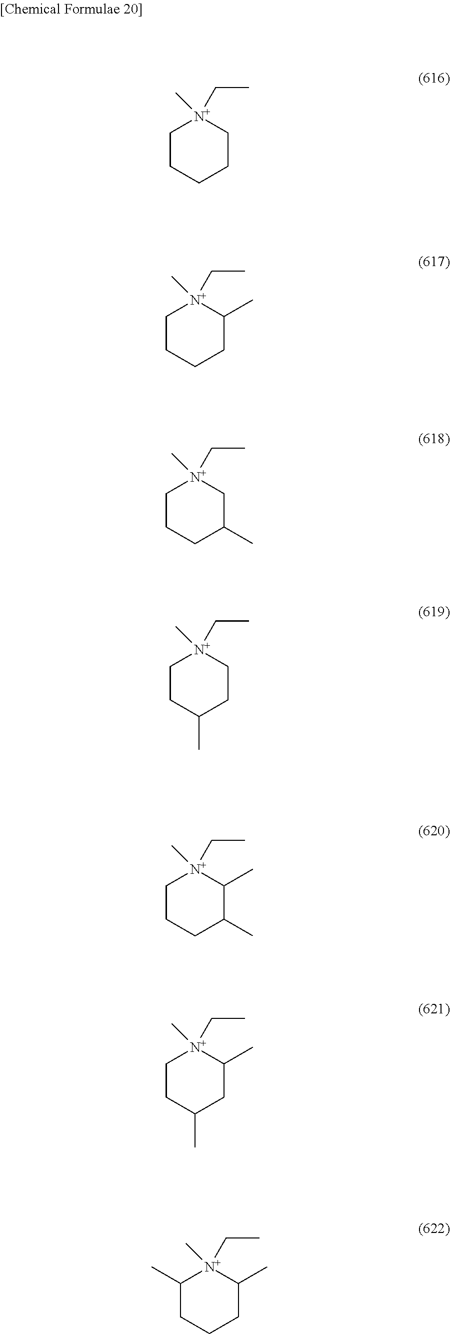

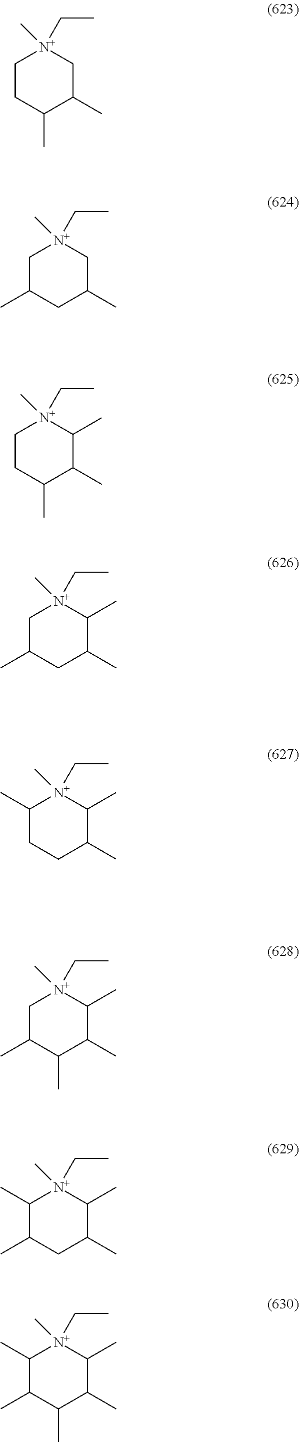

Specific examples of the cation represented by General Formula (G6) above include Structural Formula (301) to Structural Formula (309) and Structural Formula (401) to Structural Formula (419).

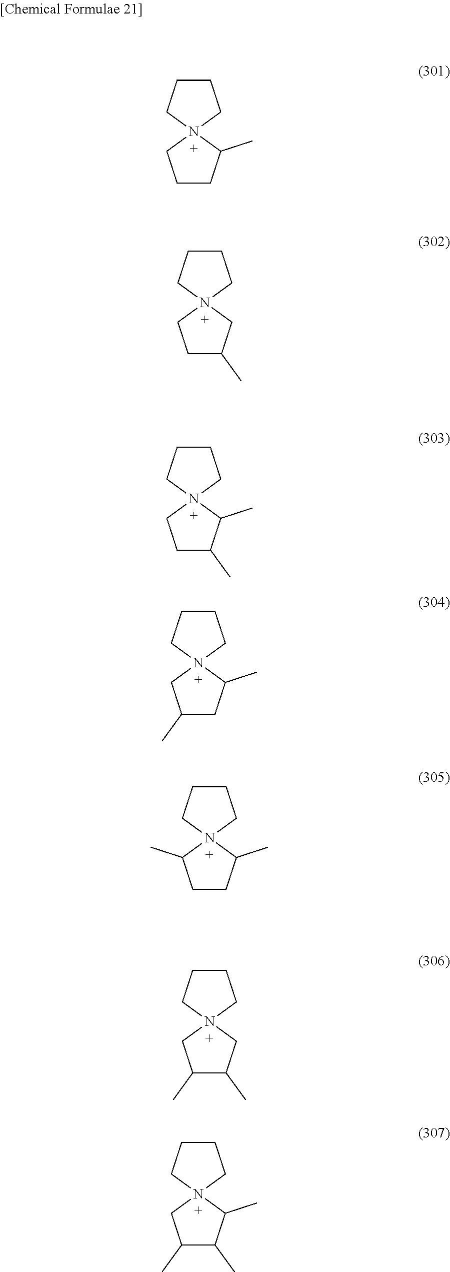

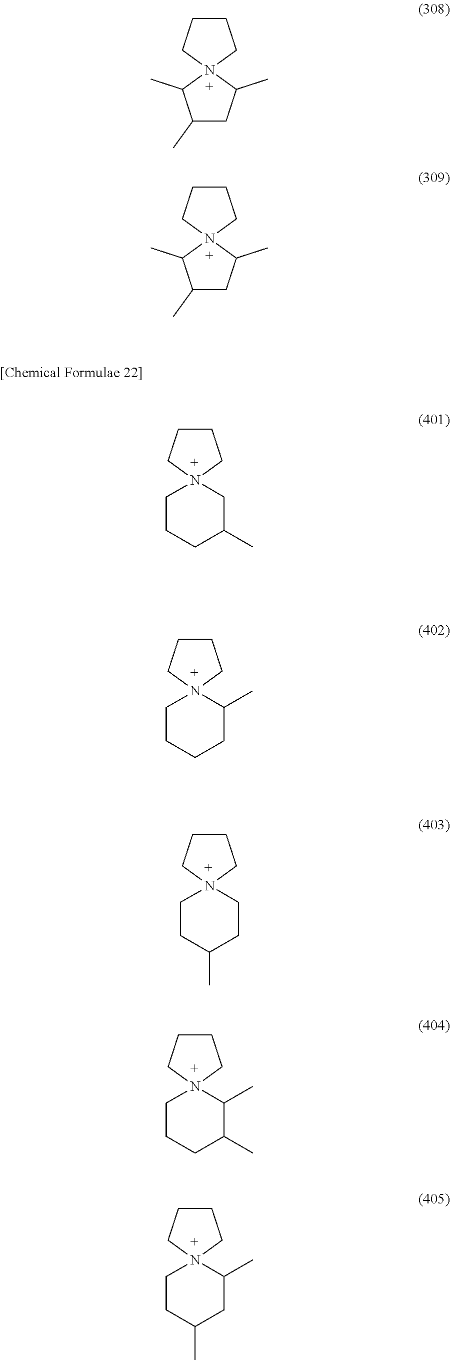

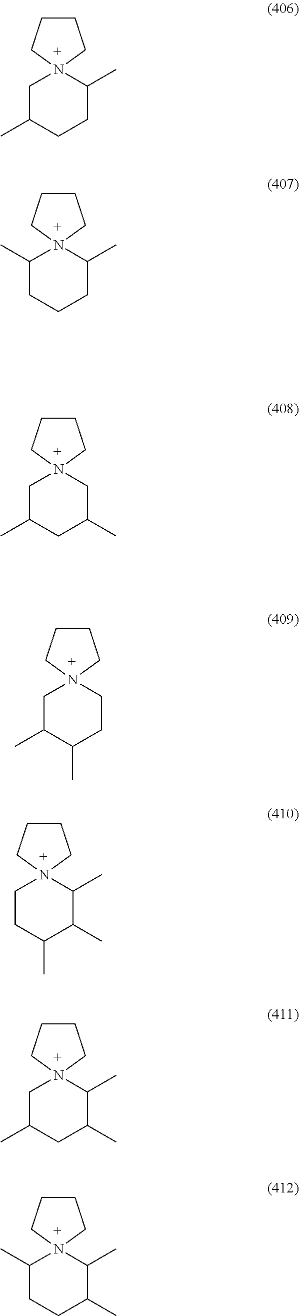

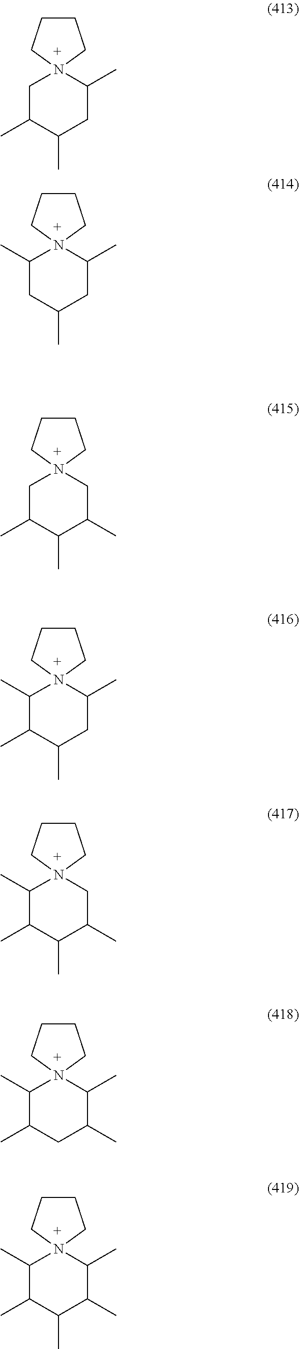

Although Structural Formula (301) to Structural Formula (309) and Structural Formula (401) to Structural Formula (419) each show an example in which m is 1 in General Formula (G6), m may be changed into 2 or 3 in Structural Formula (301) to Structural Formula (309) and Structural Formula (401) to Structural Formula (419).

Specific examples of the cation represented by General Formula (G7) above include Structural Formula (201) to Structural Formula (215).

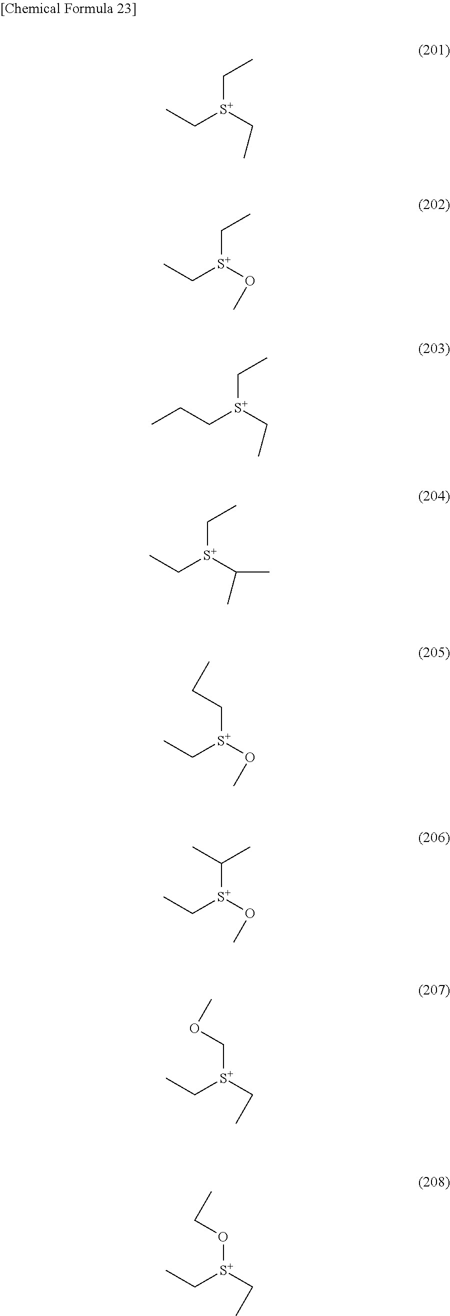

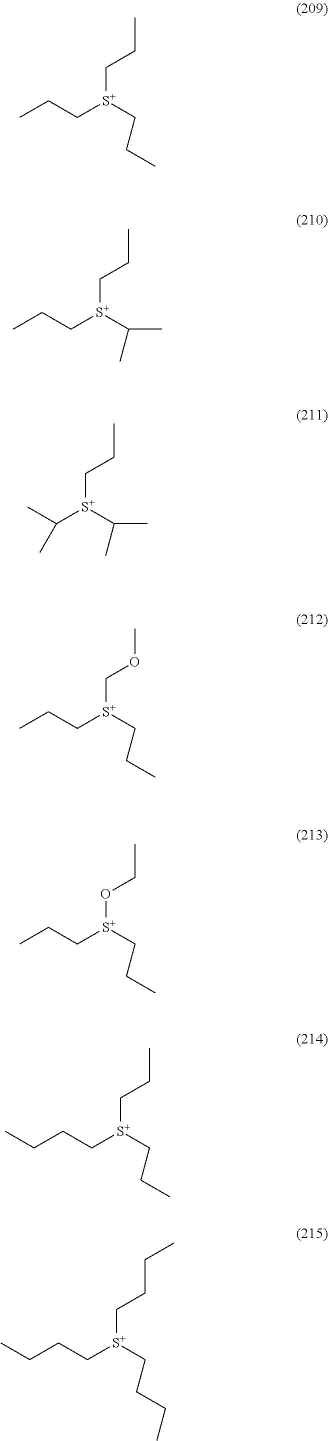

The secondary battery of one embodiment of the present invention uses the positive electrode active material of one embodiment of the present invention and an electrolyte solution containing the above-described ionic liquid, whereby a reduction in capacity can be suppressed and significantly excellent characteristics can be achieved even when the secondary battery is repeatedly used at a high charge voltage.

### [Negative Electrode Active Material]

The negative electrode of one embodiment of the present invention includes a negative electrode active material. The negative electrode of one embodiment of the present invention preferably includes a conductive agent. The negative electrode of one embodiment of the present invention preferably includes a binder.

As the negative electrode active material, a material that can react with carrier ions of the secondary battery, a material into and from which carrier ions can be inserted and extracted, a material that enables an alloying reaction with a metal serving as a carrier ion, a material that enables melting and precipitation of a metal serving as a carrier ion, or the like is preferably used.

Carbon materials such as graphite, graphitizing carbon, non-graphitizing carbon, carbon nanotube, carbon black, and graphene can be used as the negative electrode active material, for example.

In addition, a material containing one or more elements selected from silicon, tin, gallium, aluminum, germanium, lead, antimony, bismuth, silver, zinc, cadmium, and indium can be used as the negative electrode active material, for example.

An impurity element such as phosphorus, arsenic, boron, aluminum, or gallium may be added to silicon so that silicon is lowered in resistance.

As a material containing silicon, a material represented by SiOₓ, (x is preferably less than 2, further preferably greater than or equal to 0.5 and less than or equal to 1.6) can be used, for example.

A material containing silicon, which has a plurality of crystal grains in a single particle, for example, can be used. For example, a configuration where a single particle includes one or more silicon crystal grains can be used. The single particle may also include silicon oxide around the silicon crystal grain(s). The silicon oxide may be amorphous.

As a compound containing silicon, Li₂SiO₃ and Li₄SiO₄ can be used, for example. Each of Li₂SiO₃ and Li₄SiO₄ may have crystallinity, or may be amorphous.

The analysis of the compound containing silicon can be performed by NMR, XRD, a Raman spectroscopy method, or the like.

Furthermore, an oxide containing one or more elements selected from titanium, niobium, tungsten, and molybdenum can be used as a material that can be used for the negative electrode active material, for example.

As the negative electrode active material, it is possible to use a combination of two or more of the aforementioned metals, materials, compounds, and the like.

The negative electrode active material of one embodiment of the present invention may contain fluorine in a surface portion. When the negative electrode active material contains halogen in its surface portion, a decrease in charge and discharge efficiency can be suppressed. Moreover, it is considered that a reaction with an electrolyte at a surface of the active material is inhibited. In addition, at least part of the surface of the negative electrode active material of one embodiment of the present invention is covered with a region containing halogen in some cases. The region may have a film shape, for example. Fluorine is particularly preferable as halogen.

### Formation Method Example

An example of a method for forming a negative electrode active material containing halogen in its surface portion is described.

The above-described material that can be used for the negative electrode active material and a compound containing halogen are mixed as a first material and a second material, respectively, and heat treatment is performed, whereby the negative electrode active material can be formed.

In addition to the first material and the second material, a material generating eutectic reaction with the second material may be mixed as a third material. The eutectic point caused by the eutectic reaction is preferably lower than at least one of the melting point of the second material and the melting point of the third material. A decrease in the melting point due to the eutectic reaction brings the feasibility of covering the surface of the first material with the second material and the third material during the heat treatment, which increases the coverage in some cases.

As the second material and the third material, a material including a metal whose ion functions as a carrier ion in the reaction of the secondary battery is used, whereby such a metal can contribute to charging and discharging using its carrier ion, in some cases, when the metal is included in a negative electrode active material.

As the third material, a material containing oxygen and carbon can be used, for example. As the material containing oxygen and carbon, carbonate can be used, for example. Alternatively, as the material containing oxygen and carbon, an organic compound can be used, for example.

Alternatively, as the third material, hydroxide may be used.

Materials such as carbonate and hydroxide are preferable because many of them are inexpensive and have a high level of safety. Furthermore, carbonate, hydroxide, and the like generate a eutectic point with a material containing halogen, which is preferable.

More specific examples of the second material and the third material are described. When lithium fluoride is used as the second material, the lithium fluoride does not cover the surface of the first material but is aggregated only with itself, in some cases, in heating after being mixed with the first material. In such a case, a material generating a eutectic reaction with lithium fluoride is used as the third material, whereby the coverage of the surface of the first material is improved in some cases.

When the first material is heated, reaction with oxygen in an atmosphere occurs in the heating, whereby an oxide film is formed on the surface in some cases. In the formation of the negative electrode active material of one embodiment of the present invention, eutectic reaction between a material containing halogen and a material containing oxygen and carbon is caused in an annealing process described later, whereby heating at low temperatures can be performed. As a result, oxidation reaction at the surface or the like can be inhibited.

When a carbon material is used as the first material, there is a concern that carbon dioxide is generated by reaction of the carbon material and oxygen in an atmosphere in the heating to cause a reduction in the weight of the first material, damage to the surface of the first material, and the like. In the formation of the negative electrode active material of one embodiment of the present invention, the heating can be performed at a low temperature; thus, a reduction in weight, the surface damage, and the like can be inhibited even when the carbon material is used as the first material.

Here, graphite is prepared as the first material. As the graphite, flake graphite, spherical natural graphite, MCMB, or the like can be used. The surface of graphite may be covered with a low-crystalline carbon material.

As the second material, a material containing halogen is prepared. As the material containing halogen, a halogen compound containing a metal A1 can be used. As the metal A1, one or more elements selected from lithium, magnesium, aluminum, sodium, potassium, calcium, barium, lanthanum, cerium, chromium, manganese, iron, cobalt, nickel, zinc, zirconium, titanium, vanadium, and niobium can be used, for example. As the halogen compound, for example, a fluoride or a chloride can be used. The halogen contained in the material containing halogen is represented by an element Z.

Here, lithium fluoride is prepared as an example.

A material containing oxygen and carbon is prepared as the third material. As the material containing oxygen and carbon, a carbonate containing a metal A2 can be used, for example. As the metal A2, one or more selected from lithium, magnesium, aluminum, sodium, potassium, calcium, barium, lanthanum, cerium, chromium, manganese, iron, cobalt, and nickel can be used, for example.

Here, lithium carbonate is prepared as an example.

The first material, the second material, and the third material are mixed to obtain a mixture.

The second material and the third material are preferably mixed to have a ratio such that (the second material):(the third material)=a1:(1−a1) [unit: mol.] where a1 is preferably greater than 0.2 and less than 0.9, further preferably greater than or equal to 0.3 and less than or equal to 0.8.

Furthermore, the first material and the second material are preferably mixed to have a ratio such that (the first material):(the second material)=1:b1 [unit: mol.] where b1 is preferably greater than or equal to 0.001 and less than or equal to 0.2.

Next, the annealing process is performed, whereby the negative electrode active material of one embodiment of the present invention is obtained.

It is preferable that the annealing process be performed in a reduction atmosphere because the oxidation of the surface of the first material and the reaction of the first material with oxygen can be inhibited. The reduction atmosphere may be a nitrogen atmosphere or a rare gas atmosphere, for example. Furthermore, two or more types of gases selected from nitrogen and a rare gas may be mixed and used. The heating may be performed under reduced pressure.

In the case where the melting point of the second material is represented by M₂ [° C.], the heating temperature is preferably higher than (M₂−550) [K] and lower than (M₂+50) [K], further preferably higher than or equal to (M₂−400) [° C.] and lower than or equal to (M₂) [° C.].

Moreover, in a compound, solid-phase diffusion occurs easily at a temperature higher than or equal to the Tamman temperature. The Tamman temperature of an oxide, for example, is 0.757 times of the melting point. Thus, the heating temperature is preferably higher than or equal to 0.757 times of the melting point or higher than its vicinity, for example.

In the case of lithium fluoride that is a typical example of the material containing halogen, the amount of evaporation increases rapidly at a temperature higher than or equal to the melting point. Thus, the heating temperature is preferably lower than or equal to the melting point of the material containing halogen, for example.

In the case where the eutectic point of the second material and the third material is represented by M₂₃ [K], the heating temperature is, for example, preferably higher than (M₂₃×0.7) [K] and lower than (M₂+50) [K], preferably higher than or equal to (M₂₃×0.75) [K] and lower than or equal to (M₂+20) [K], preferably higher than or equal to (M₂₃×0.75) [K] and lower than or equal to (M₂+20) [K], preferably higher than M₂₃ [K] and lower than (M₂+10) [K], further preferably higher than or equal to (M₂₃×0.8) [K] and lower than or equal to M₂ [K], further preferably higher than or equal to (M₂₃) [K] and lower than or equal to M₂ [K].

In the case where lithium fluoride is used as the second material and lithium carbonate is used as the third material, the heating temperature is, for example, preferably higher than 350° C. and lower than 900° C., further preferably higher than or equal to 390° C. and lower than or equal to 850° C., still further preferably higher than or equal to 520° C. and lower than or equal to 910° C., still further preferably higher than or equal to 570° C. and lower than or equal to 860° C., yet still further preferably higher than or equal to 610° C. and lower than or equal to 860° C.

The heating time is preferably longer than or equal to 1 hour and shorter than or equal to 60 hours, further preferably longer than or equal to 3 hours and shorter than or equal to 20 hours, for example.

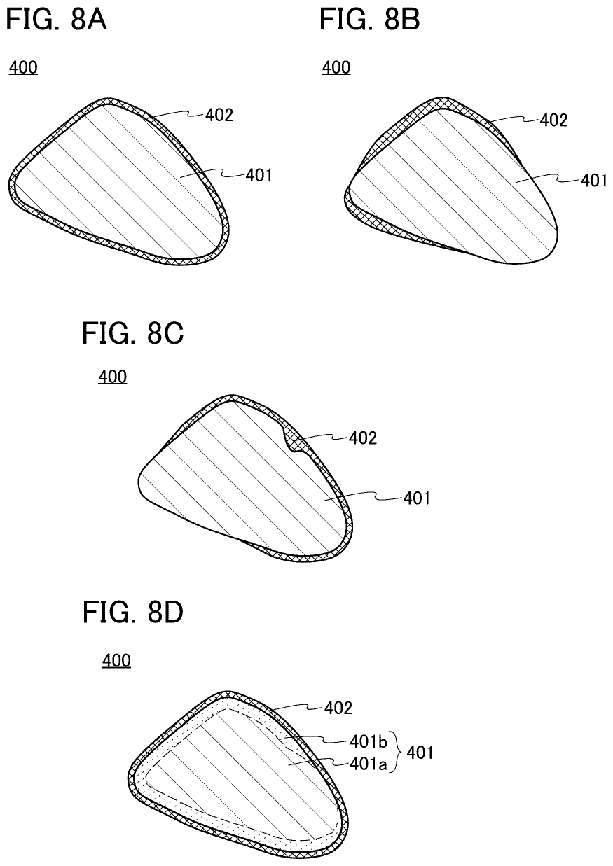

FIG. 8A, FIG. 8B, FIG. 8C, and FIG. 8D each show an example of a cross section of a negative electrode active material 400.

The cross section of the negative electrode active material 400 is exposed by processing, whereby observation and analysis of the cross section can be performed.

The negative electrode active material 400 illustrated in FIG. 8A includes a region 401 and a region 402. The region 402 is positioned on an outer side of the region 401. The region 402 is preferably in contact with the surface of the region 401.

At least part of the region 402 preferably includes the surface of the negative electrode active material 400.

The region 401 is, for example, a region including an inner portion of the negative electrode active material 400.

The region 401 includes the first material described above. The region 402 includes the element Z, oxygen, carbon, the metal A1, and the metal A2, for example. The element Z is, for example, fluorine or chlorine. The region 402 does not include some of elements of the element Z, oxygen, carbon, the metal A1, and the metal A2, in some cases. Alternatively, in the region 402, some of the elements of the element Z, oxygen, carbon, the metal A1, and the metal A2 have low concentration and are not detected by analysis in some cases.

The region 402 is called a surface portion of the negative electrode active material 400 or the like, in some case.

The negative electrode active material 400 can have a variety of forms such as one particle, a group of a plurality of particles, and a thin film.

The region 401 may be a particle of the first material. Alternatively, the region 401 may be a group of a plurality of particles of the first material. Alternatively, the region 401 may be a thin film of the first material.

The region 402 may be part of a particle. For example, the region 402 may be a surface portion of the particle. Alternatively, the region 402 may be part of a thin film. For example, the region 402 may be an upper layer portion of a thin film.

The region 402 may be a coating layer formed on the surface of the particle.

The region 402 may be a region including a bond of a constituent element of the first material and the element Z. For example, in the region 402 or the interface between the region 401 and the region 402, the surface of the first material may be modified with the element Z or a functional group including the element Z. Thus, in the negative electrode active material of one embodiment of the present invention, the bond of a constituent element of the first material and the element Z is observed in some cases. As an example, in the case where the first material is graphite and the element Z is fluorine, a C—F bond is, for example, observed in some cases. As another example, in the case where the first material contains silicon and the element Z is fluorine, a Si—F bond is, for example, observed in some cases.

For example, in the case where graphite is used as the first material, the region 401 is a graphite particle, and the region 402 is a coating layer of the graphite particle. As another example, in the case where graphite is used as the first material, the region 401 is a region including an inner portion of a graphite particle, and the region 402 is a surface portion of the graphite particle.

The region 402 includes, for example, a bond of the element Z and carbon. The region 402 includes, for example, a bond of the element Z and the metal A1. The region 402 includes, for example, a carbonate group.

When the negative electrode active material 400 is analyzed by X-ray photoelectron spectroscopy (XPS), the element Z is preferably detected, in which case the concentration of the detected element Z is preferably higher than or equal to 1 atomic %. In this case, the concentration of the element Z can be calculated on the assumption that the total of concentrations of carbon, oxygen, the metal A1, the metal A2, and the element Z is 100%, for example. Alternatively, the calculation may be performed on the assumption that the value obtained by adding the nitrogen concentration to the concentrations of the above elements is set as 100%. The concentration of the element Z is, for example, lower than or equal to 60 atomic %, or for example, lower than or equal to 30 atomic %.

When the negative electrode active material 400 is analyzed by XPS, a peak attributed to the bond of the element Z and carbon is preferably detected. A peak attributed to the bond of the element Z and the metal A1 may be detected.

In the case where the element Z is fluorine and the metal A1 is lithium, in the F1s spectrum by XPS, a peak indicating the carbon-fluorine bond (hereinafter, a peak F2) is observed in the vicinity of 688 eV (e.g., its peak position is observed in an energy range higher than 686.5 eV and lower than 689.5 eV), and a peak indicating the lithium-fluorine bond (hereinafter, a peak F1) is observed in the vicinity of 685 eV (e.g., its peak position is observed in an energy range higher than 683.5 eV and lower than 686.5 eV). The intensity of the peak F2 is preferably higher than 0.1 times the intensity of the peak F1 and lower than 10 times the intensity of the peak F1. For example, the intensity of the peak F2 is higher than or equal to 0.3 times the intensity of the peak F1 and lower than or equal to 3 times the intensity of the peak F1.

When the negative electrode active material 400 is analyzed by XPS, a peak corresponding to carbonate or a carbonate group is preferably observed. In the C1s spectrum by XPS, the peak corresponding to carbonate or a carbonate group is observed in the vicinity of 290 eV (e.g., its peak position is observed in an energy range higher than 288.5 eV and lower than 291.5 eV).

In XRD analysis of the negative electrode active material 400, a spectrum derived from Li₂O represented by a space group Fm-3m is observed in some cases.

In the example shown in FIG. 8B, the region 401 includes a region not covered with the region 402. In the example shown in FIG. 8C, the region 402 covering a region depressed at the surface of the region 401 has a large thickness.

In the negative electrode active material 400 illustrated in FIG. 8D, the region 401 includes a region 401a and a region 401b. The region 401a is a region including the inner portion of the region 401, and the region 401b is positioned on an outer side of the region 401a. In addition, the region 401b is preferably in contact with the region 402.

The region 401b is a surface portion of the region 401.

The region 401b contains one or more elements of the element Z, oxygen, carbon, the metal A1, and the metal A2 contained in the region 402. In the region 401b, the elements contained in the region 402, such as the element Z, oxygen, carbon, the metal A1, and the metal A2, may have a concentration gradient such that the concentration decreases gradually from the surface or the vicinity of the surface to the inner portion.

The concentration of the element Z contained in the region 401b is higher than the concentration of the element Z contained in the region 401a. The concentration of the element Z contained in the region 401b is preferably lower than the concentration of the element Z contained in the region 402.

The concentration of oxygen contained in the region 401b is higher than the concentration of oxygen contained in the region 401a in some cases. The concentration of oxygen contained in the region 401b is lower than the concentration of oxygen contained in the region 402 in some cases.

When the negative electrode active material of one embodiment of the present invention is measured by energy dispersive X-ray spectroscopy using a scanning electron microscope, it is preferable that the element Z be detected. For example, the concentration of the element Z is preferably higher than or equal to 10 atomic % and lower than or equal to 70 atomic % on the assumption that the total of the concentrations of the element Z and oxygen is 100 atomic %.

The region 402 has a region whose thickness is smaller than or equal to 50 nm, preferably larger than or equal to 1 nm and smaller than or equal to 35 nm, further preferably larger than or equal to 5 nm and smaller than or equal to 20 nm, for example.

The region 401b has a region whose thickness is smaller than or equal to 50 nm, preferably larger than or equal to 1 nm and smaller than or equal to 35 nm, further preferably larger than or equal to 5 nm and smaller than or equal to 20 nm, for example.

In the case where fluorine is used as the element Z and lithium is used as the metal A1 and the metal A2, the region 402 may include a region covered with a region containing lithium fluoride and a region covered with a region containing lithium carbonate, with respect to the region 401. The region 402 does not obstruct the insertion and extraction of lithium and accordingly enables an excellent secondary battery to be achieved without a degradation of output characteristics or the like of the secondary battery.

This embodiment can be combined with the description of the other embodiments as appropriate.

### Embodiment 2

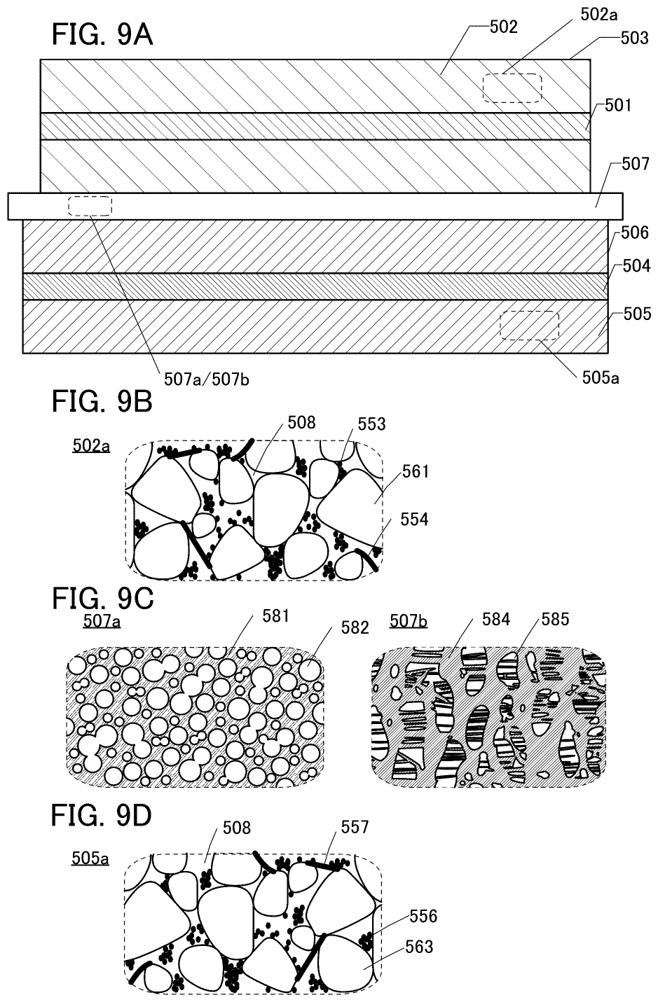

In this embodiment, an example of a secondary battery of one embodiment of the present invention is described with reference to FIG. 9. The secondary battery includes an exterior body (not illustrated), a positive electrode 503, a negative electrode 506, a separator 507, and an electrolyte 508 in which a lithium salt or the like is dissolved. The separator 507 is provided between the positive electrode 503 and the negative electrode 506.

The positive electrode of one embodiment of the present invention includes a positive electrode active material layer. The positive electrode active material layer contains a positive electrode active material. The positive electrode active material layer may include a conductive agent, a binder, and the like. The positive electrode of one embodiment of the present invention preferably includes a current collector, and the positive electrode active material layer is preferably provided over the current collector.

In FIG. 9, the positive electrode 503 includes a positive electrode active material layer 502 and a positive electrode current collector 501. The positive electrode active material layer 502 includes a positive electrode active material 561, a conductive additive, and a binder. FIG. 9B is an enlarged view of a region 502a illustrated in FIG. 9A. FIG. 9B shows an example of using acetylene black 553 and graphene 554 as conductive agents.

The negative electrode of one embodiment of the present invention includes a negative electrode active material layer. The negative electrode active material layer contains a negative electrode active material. The negative electrode active material layer may include a conductive agent, a binder, and the like. The negative electrode of one embodiment of the present invention preferably includes a current collector, and the negative electrode active material layer is preferably provided over the current collector.

The negative electrode 506 includes a negative electrode active material layer 505 and a negative electrode current collector 504. The negative electrode active material layer 505 includes a negative electrode active material 563, a conductive agent, and a binder. FIG. 9D is an enlarged view of a region 505a illustrated in FIG. 9A. FIG. 9D shows an example of using acetylene black 556 and graphene 557 as conductive agents.

As the conductive agent, a carbon material, a metal material, a conductive ceramic material, or the like can be used. Alternatively, a fiber material may be used as the conductive agent. The content of the conductive agent to the total amount of the active material layer is preferably greater than or equal to 1 wt % and less than or equal to 10 wt %, further preferably greater than or equal to 1 wt % and less than or equal to 5 wt %.

A network for electric conduction can be formed in the active material layer by the conductive agent. The conductive agent also allows maintaining of a path for electric conduction between the active materials. The addition of the conductive agent to the active material layer increases the electric conductivity of the active material layer.

As the conductive agent, a graphene compound can be used. Moreover, natural graphite, artificial graphite such as mesocarbon microbeads, carbon fiber, or the like can be used as the conductive agent.

As carbon fiber, carbon fiber such as mesophase pitch-based carbon fiber or isotropic pitch-based carbon fiber can be used, for example. As the carbon fiber, carbon nanofiber, carbon nanotube, or the like can be used. Carbon nanotube can be formed by, for example, a vapor deposition method. Other examples of the conductive agent include carbon materials such as carbon black (e.g., acetylene black (AB)), graphite (black lead) particles, graphene, and fullerene. Alternatively, one or more selected from metal powder and metal fiber of copper, nickel, aluminum, silver, gold, or the like, a conductive ceramic material, and the like can be used.

### [Graphene Compound]

A graphene compound in this specification and the like refers to multilayer graphene, multi graphene, graphene oxide, multilayer graphene oxide, multi graphene oxide, reduced graphene oxide, reduced multilayer graphene oxide, reduced multi graphene oxide, graphene quantum dots, and the like. A graphene compound contains carbon, has a plate-like shape, a sheet-like shape, or the like, and has a two-dimensional structure formed of a six-membered ring composed of carbon atoms. The two-dimensional structure formed of the six-membered ring composed of carbon atoms may be referred to as a carbon sheet. A graphene compound may include a functional group. A graphene compound is preferably bent. A graphene compound may be rounded like carbon nanofiber.

As the conductive agent, it is possible to use a combination of the above-described materials.

In this specification and the like, graphene oxide contains carbon and oxygen, has a sheet-like shape, and includes a functional group, in particular, an epoxy group, a carboxy group, or a hydroxy group.

In this specification and the like, reduced graphene oxide contains carbon and oxygen, has a sheet-like shape, and has a two-dimensional structure formed of a six-membered ring composed of carbon atoms. The reduced graphene oxide may also be referred to as a carbon sheet. The reduced graphene oxide functions by itself and may have a stacked-layer structure. The reduced graphene oxide preferably includes a portion where the carbon concentration is higher than 80 atomic % and the oxygen concentration is higher than or equal to 2 atomic % and lower than or equal to 15 atomic %. With such a carbon concentration and such an oxygen concentration, the reduced graphene oxide can function as a conductive agent with high conductivity even with a small amount. In addition, the intensity ratio G/D of a G band to a D band of the Raman spectrum of the reduced graphene oxide is preferably 1 or more. The reduced graphene oxide with such an intensity ratio can function as a conductive agent with high conductivity even with a small amount.

In the longitudinal cross section of the active material layer, the sheet-like graphene compounds are dispersed substantially uniformly in a region inside the active material layer. The plurality of graphene compounds are formed to partly coat a plurality of particles of the active material or adhere to the surfaces of the plurality of particles of the active material, so that the graphene compounds make surface contact with the particles of the active material.

Here, the plurality of graphene compounds can be bonded to each other to form a net-like graphene compound sheet (hereinafter, referred to as a graphene compound net or a graphene net). A graphene net that covers the active material can function as a binder for bonding the active materials. Accordingly, the amount of the binder can be reduced, or the binder does not have to be used. This can increase the proportion of the active material in the electrode volume and the electrode weight. That is, the charge and discharge capacity of the secondary battery can be increased.

Here, it is preferable to perform reduction after a layer to be the active material layer is formed in such a manner that graphene oxide is used as the graphene compound and mixed with an active material. That is, the formed active material layer preferably contains reduced graphene oxide. When graphene oxide with extremely high dispersibility in a polar solvent is used to form the graphene compounds, the graphene compounds can be substantially uniformly dispersed in a region inside the active material layer. The solvent is removed by volatilization from a dispersion medium containing the uniformly dispersed graphene oxide to reduce the graphene oxide; hence, the graphene compounds remaining in the active material layer partly overlap with each other and are dispersed such that surface contact is made, thereby forming a three-dimensional conductive path. Note that graphene oxide may be reduced by heat treatment or with the use of a reducing agent, for example. Unlike a particulate conductive agent such as acetylene black, which makes point contact with an active material, the graphene compound is capable of making low-resistance surface contact; accordingly, the electric conduction in the electrode can be improved with a smaller amount of the graphene compound than that of a normal conductive agent. This can increase the proportion of the active material in the active material layer. Thus, discharge capacity of the secondary battery can be increased.

### [Binder]

As the binder, for example, a rubber material such as styrene-butadiene rubber (SBR), styrene-isoprene-styrene rubber, acrylonitrile-butadiene rubber, butadiene rubber, or an ethylene-propylene-diene copolymer is preferably used. Alternatively, fluororubber can be used as the binder.

As the binder, for example, water-soluble polymers are preferably used. As the water-soluble polymers, a polysaccharide can be used, for example. As the polysaccharide, one or more selected from starch, cellulose derivatives such as carboxymethyl cellulose (CMC), methyl cellulose, ethyl cellulose, hydroxypropyl cellulose, diacetyl cellulose, and regenerated cellulose, and the like can be used. It is further preferred that such water-soluble polymers be used in combination with any of the above-described rubber materials.

Alternatively, as the binder, a material such as polystyrene, poly(methyl acrylate), poly(methyl methacrylate) (PMMA), sodium polyacrylate, polyvinyl alcohol (PVA), polyethylene oxide (PEO), polypropylene oxide, polyimide, polyvinyl chloride, polytetrafluoroethylene, polyethylene, polypropylene, polyisobutylene, polyethylene terephthalate, nylon, polyvinylidene fluoride (PVDF), polyacrylonitrile (PAN), an ethylene-propylene-diene polymer, polyvinyl acetate, or nitrocellulose is preferably used.

Two or more of the above materials may be used in combination for the binder.

For example, a material having a significant viscosity modifying effect and another material may be used in combination. For example, a rubber material or the like has high adhesion and high elasticity but may have difficulty in viscosity modification when mixed in a solvent. In such a case, a rubber material or the like is preferably mixed with a material having a significant viscosity modifying effect, for example. As a material having a significant viscosity modifying effect, for instance, a water-soluble polymer is preferably used. As a water-soluble polymer having a significant viscosity modifying effect, one or more selected from the above-mentioned polysaccharides, for instance, starch and cellulose derivatives such as carboxymethyl cellulose (CMC), methyl cellulose, ethyl cellulose, hydroxypropyl cellulose, diacetyl cellulose, and regenerated cellulose can be used.

Note that a cellulose derivative such as carboxymethyl cellulose obtains a higher solubility when converted into a salt such as a sodium salt or an ammonium salt of carboxymethyl cellulose, and thus easily exerts an effect as a viscosity modifier. A high solubility can also increase the dispersibility of an active material and other components in the formation of slurry for an electrode. In this specification, cellulose and a cellulose derivative used as a binder of an electrode include salts thereof.

A water-soluble polymer stabilizes the viscosity by being dissolved in water and allows stable dispersion of the active material and another material combined as a binder, such as styrene-butadiene rubber, in an aqueous solution. Furthermore, a water-soluble polymer is expected to be easily and stably adsorbed onto an active material surface because it has a functional group. Many cellulose derivatives, such as carboxymethyl cellulose, have a functional group such as a hydroxyl group or a carboxyl group. Because of functional groups, polymers are expected to interact with each other and cover an active material surface in a large area.

In the case where the binder that covers or is in contact with the active material surface forms a film, the film is expected to serve also as a passivation film to suppress the decomposition of the electrolyte solution. Here, a passivation film refers to a film without electric conductivity or a film with extremely low electric conductivity, and can inhibit the decomposition of an electrolyte solution at a potential at which a battery reaction occurs when the passivation film is formed on the active material surface, for example. It is preferred that the passivation film can conduct lithium ions while suppressing electric conduction.

The active material layer can be formed in such a manner that an active material, a binder, a conductive additive, and a solvent are mixed to form slurry, the slurry is formed over a current collector, and the solvent is volatilized.

A solvent used for the slurry is preferably a polar solvent. For example, water, methanol, ethanol, acetone, tetrahydrofuran (THF), dimethylformamide (DMF), N-methylpyrrolidone (NMP), dimethyl sulfoxide (DMSO), or a mixed solution of two or more of the above can be used.

### [Current Collector]

For each of a positive electrode current collector and a negative electrode current collector, it is possible to use a material which has high conductivity and is not alloyed with carrier ions such as lithium, e.g., a metal such as stainless steel, gold, platinum, zinc, iron, copper, aluminum, or titanium, an alloy thereof, or the like. It is also possible to use an aluminum alloy to which an element that improves heat resistance, such as silicon, titanium, neodymium, scandium, or molybdenum, is added. Alternatively, a metal element that forms silicide by reacting with silicon may be used. Examples of the metal element that forms silicide by reacting with silicon include zirconium, titanium, hafnium, vanadium, niobium, tantalum, chromium, molybdenum, tungsten, cobalt, and nickel. The current collector can have a sheet-like shape, a net-like shape, a punching-metal shape, an expanded-metal shape, or the like as appropriate. The thickness of the current collector is preferably larger than or equal to 10 μm and smaller than or equal to 30 μm.

Note that a material that is not alloyed with carrier ions of lithium or the like is preferably used for the negative electrode current collector.

As each of the current collectors, a titanium compound may be stacked over the above-described metal element. As a titanium compound, for example, it is possible to use one selected from titanium nitride, titanium oxide, titanium nitride in which oxygen is substituted for part of nitrogen, titanium oxide in which nitrogen is substituted for part of oxygen, and titanium oxynitride (TiOₓN_{y}, where 0<x<2 and 0<y<1), or a mixture or a stack of two or more of them. Titanium nitride is particularly preferable because it has high conductivity and has a high capability of inhibiting oxidation. Provision of a titanium compound over the surface of the current collector inhibits a reaction between a material contained in the active material layer formed over the current collector and the metal, for example. In the case where the active material layer contains a compound containing oxygen, an oxidation reaction between the metal element and oxygen can be inhibited. In the case where aluminum is used for the current collector and the active material layer is formed using graphene oxide described later, for example, an oxidation reaction between oxygen contained in the graphene oxide and aluminum might occur. In such a case, provision of a titanium compound over aluminum can inhibit an oxidation reaction between the current collector and the graphene oxide.

As each of the graphene 554 and the graphene 557, graphene or a graphene compound can be used.

A graphene compound in this specification and the like refers to multilayer graphene, multi graphene, graphene oxide, multilayer graphene oxide, multi graphene oxide, reduced graphene oxide, reduced multilayer graphene oxide, reduced multi graphene oxide, graphene quantum dots, and the like. A graphene compound contains carbon, has a plate-like shape, a sheet-like shape, or the like, and has a two-dimensional structure formed of a six-membered ring composed of carbon atoms. The two-dimensional structure formed of the six-membered ring composed of carbon atoms may be referred to as a carbon sheet. A graphene compound may include a functional group. A graphene compound is preferably bent. A graphene compound may be rounded like carbon nanofiber.

In the positive electrode or the negative electrode of one embodiment of the present invention, graphene or a graphene compound can function as a conductive agent. A plurality of sheets of graphene or graphene compounds form a three-dimensional conductive path in the positive electrode or the negative electrode and can increase the conductivity of the positive electrode or the negative electrode. Because the graphene or graphene compounds can cling to the particles in the positive electrode or the negative electrode, the break of the particles in the positive electrode or the negative electrode can be suppressed and the strength of the positive electrode or the negative electrode can be increased. The graphene or graphene compound has a thin sheet-like shape and can form the excellent conductive path even though occupying a small volume in the positive electrode or the negative electrode, whereby the volume of the active material in the positive electrode or the negative electrode can be increased and the capacity of the secondary battery can be increased. Therefore, the capacity of the secondary battery can be increased.

### [Separator]

The separator 507 can be formed using paper, nonwoven fabric, glass fiber, ceramics, or the like. Alternatively, the separator 507 can be formed using nylon (polyamide), vinylon (polyvinyl alcohol-based fiber), polyester, acrylic, polyolefin, polyurethane, polypropylene, polyethylene, or the like. The separator is preferably formed to have an envelope-like shape to wrap one of the positive electrode and the negative electrode.

For the separator 507, for example, a polymer film including polypropylene, polyethylene, polyimide, or the like can be used. Owing to its high wettability with respect to an ionic liquid, polyimide may be further preferable as a material of the separator 507.

A polymer film including polypropylene, polyethylene, or the like can be formed by a dry method or a wet method. The dry method is a method in which a polymer film including polypropylene, polyethylene, polyimide, or the like is stretched while being heated so that a space is formed between crystals, whereby a minute hole is formed. The wet method is a method in which a resin to which a solvent is mixed in advance is processed into a film and then the solvent is extracted, whereby a hole is formed.

On the left side of FIG. 9C, an enlarged view of a region 507a illustrated in FIG. 9A is shown as an example of the separator 507 (formed by the wet method). This example shows a structure in which a plurality of holes 582 are formed in a polymer film 581. On the right side of FIG. 9C, an enlarged view of a region 507b is shown as another example of the separator 507 (formed by the dry method). This example shows a structure in which a plurality of holes 585 are formed in a polymer film 584.

After charging and discharging, the diameter of the hole in the separator may differ between a surface portion of a surface that faces the positive electrode and a surface portion of a surface that faces the negative electrode. In this specification and the like, a surface portion of the separator is preferably a region that is less than or equal to 5 μm, further preferably less than or equal to 3 μm from the surface, for example.

The separator may have a multilayer structure. For example, a structure in which two kinds of polymer materials are stacked may be employed.

For example, it is possible to employ a structure in which a polymer film including polypropylene, polyethylene, polyimide, or the like is coated with a ceramic-based material, a fluorine-based material, a polyamide-based material, a mixture thereof, or the like. Alternatively, for example, it is possible to employ a structure in which nonwoven fabric is coated with a ceramic-based material, a fluorine-based material, a polyamide-based material, a mixture thereof, or the like. Owing to its high wettability with respect to an ionic liquid, polyimide may be further preferable as a material to be coated.

Examples of the fluorine-based material include PVDF and polytetrafluoroethylene.

Examples of the polyamide-based material include nylon and aramid (meta-based aramid and para-based aramid).

### [Exterior Body]

For an exterior body included in the secondary battery, one or more selected from a metal material such as aluminum and a resin material can be used, for example. Alternatively, a film-like exterior body can also be used. As the film, for example, it is possible to use a film having a three-layer structure in which a highly flexible metal thin film of aluminum, stainless steel, copper, nickel, or the like is provided over a film formed of a material such as polyethylene, polypropylene, polycarbonate, ionomer, or polyamide, and an insulating synthetic resin film of a polyamide-based resin, a polyester-based resin, or the like is provided over the metal thin film as the outer surface of the exterior body.

This embodiment can be combined with the other embodiments as appropriate.

### Embodiment 3

In this embodiment, a method for manufacturing a secondary battery will be described.

### <Manufacturing Method 1 of Laminated Secondary Battery>

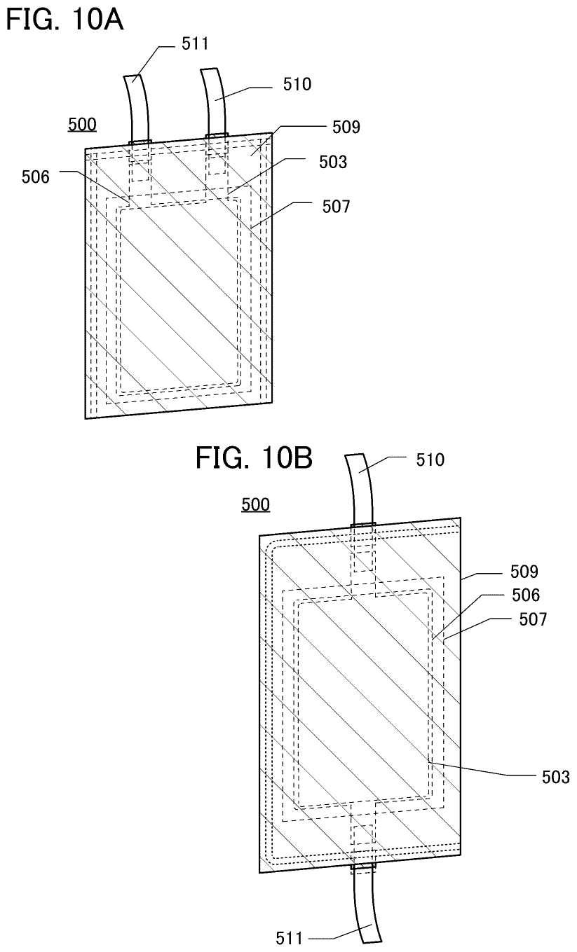

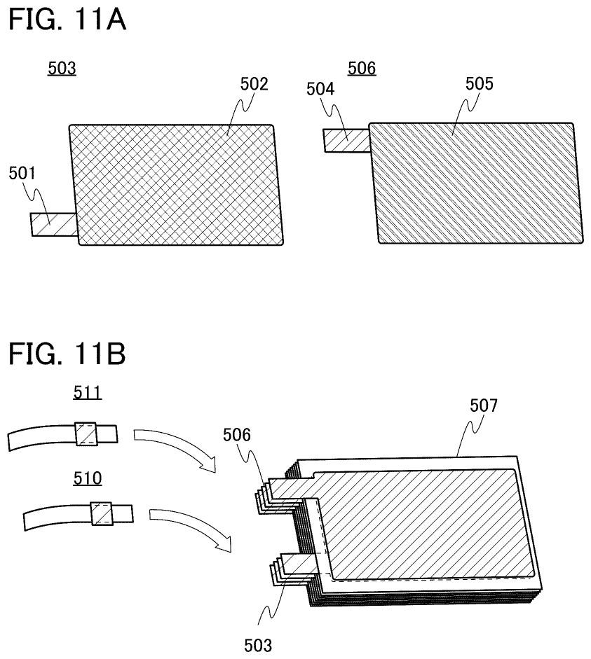

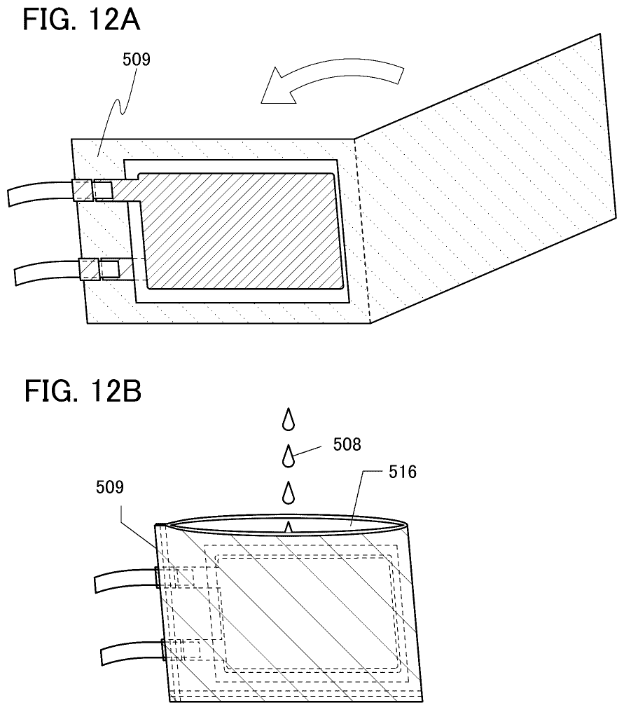

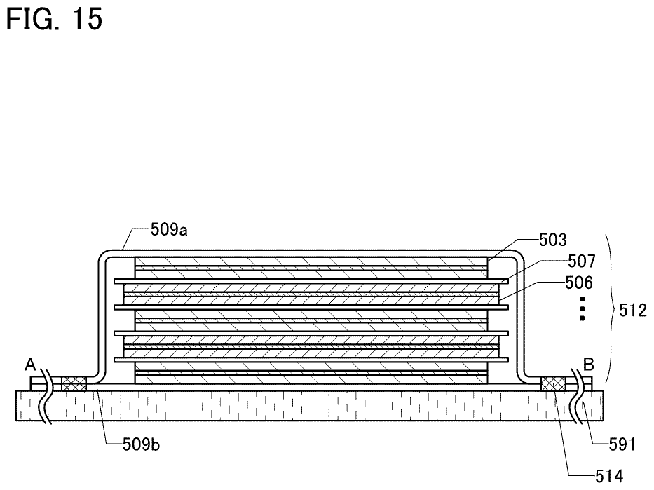

Here, an example of a method for manufacturing laminated secondary batteries whose external views are shown in FIG. 10A and FIG. 10B will be described with reference to FIG. 11A and FIG. 11B and FIG. 12A and FIG. 12B. Secondary batteries 500 illustrated in FIG. 10A and FIG. 10B each include the positive electrode 503, the negative electrode 506, the separator 507, an exterior body 509, a positive electrode lead electrode 510, and a negative electrode lead electrode 511. Note that as a cross-sectional structure of the laminated secondary battery illustrated in FIG. 10A or the like, for example, it is possible to employ a structure in which a stack including the positive electrodes, the separators, and the negative electrodes is surrounded by exterior bodies as illustrated in FIG. 15 described later.

First, the positive electrode 503, the negative electrode 506, and the separator 507 are prepared. FIG. 11A shows examples of the positive electrode 503 and the negative electrode 506. The positive electrode 503 includes the positive electrode active material layer 502 over the positive electrode current collector 501. The positive electrode 503 preferably includes a tab region where the positive electrode current collector 501 is exposed. The negative electrode 506 includes the negative electrode active material layer 505 over the negative electrode current collector 504. The negative electrode 506 preferably includes a tab region where the negative electrode current collector 504 is exposed.

Next, the negative electrode 506, the separator 507, and the positive electrode 503 are stacked. FIG. 11B illustrates the negative electrodes 506, the separators 507, and the positive electrodes 503 that are stacked. Here, an example in which 5 negative electrodes and 4 positive electrodes are used is shown. The component can also be referred to as a stack including the negative electrodes, the separators, and the positive electrodes.

Then, the tab regions of the positive electrodes 503 are bonded to each other, and the positive electrode lead electrode 510 is bonded to the tab region of the positive electrode on the outermost surface. The bonding is performed by ultrasonic welding, for example. In a similar manner, the tab regions of the negative electrodes 506 are bonded to each other, and the negative electrode lead electrode 511 is bonded to the tab region of the negative electrode on the outermost surface.

Next, the negative electrode 506, the separator 507, and the positive electrode 503 are placed over the exterior body 509.

Subsequently, the exterior body 509 is folded along a portion shown by a dashed line as illustrated in FIG. 12A. Then, the outer edges of the exterior body 509 are bonded to each other. The bonding is performed by thermocompression bonding, for example. At this time, an unbonded region (hereinafter referred to as an inlet 516) is provided for part (or one side) of the exterior body 509 so that an electrolyte 508 can be introduced later.

Next, as illustrated in FIG. 12B, the electrolyte 508 is introduced into the exterior body 509 from the inlet 516 of the exterior body 509. The electrolyte 508 is preferably introduced in a reduced-pressure atmosphere or in an inert atmosphere. Lastly, the inlet 516 is bonded. In the above manner, the laminated secondary battery 500 can be manufactured.

In the above, the positive electrode lead electrode 510 and the negative electrode lead electrode 511 on the same side are led out to the outside of the exterior body, whereby the secondary battery 500 illustrated in FIG. 10A is manufactured. The positive electrode lead electrode 510 and the negative electrode lead electrode 511 on opposite sides are led out to the outside of the exterior body, whereby the secondary battery 500 illustrated in FIG. 10B can be manufactured.

### <Manufacturing Method 2 of Laminated Secondary Battery>

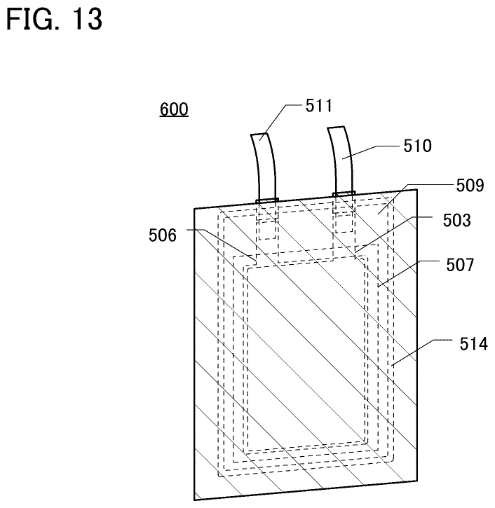

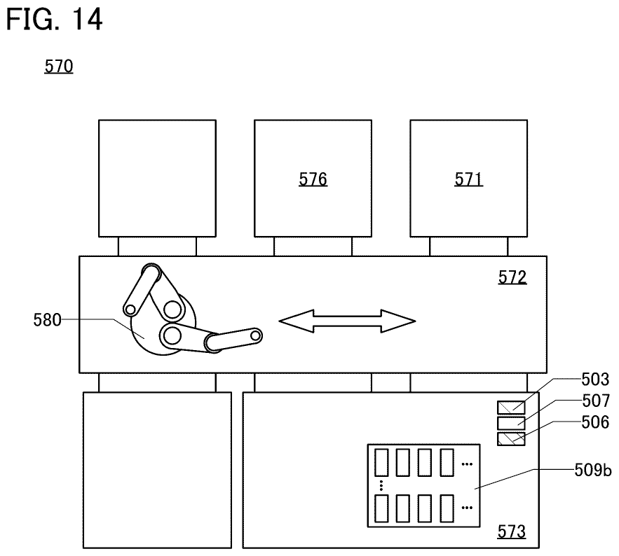

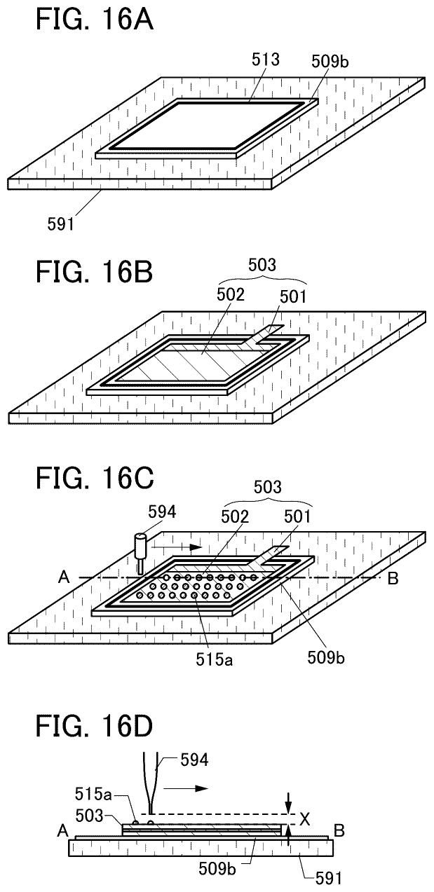

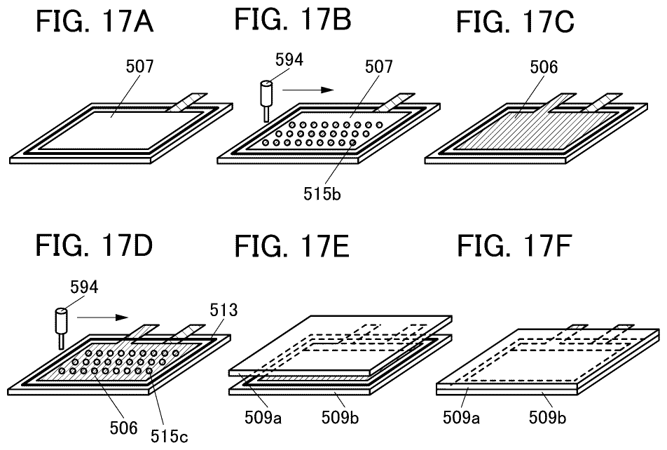

Next, an example of a method for manufacturing a laminated secondary battery 600 whose external view is shown in FIG. 13 will be described with reference to FIG. 14, FIG. 15, FIG. 16A to FIG. 16D, and FIG. 17A to FIG. 17F. The secondary battery 600 illustrated in FIG. 13 includes the positive electrode 503, the negative electrode 506, the separator 507, the exterior body 509, the positive electrode lead electrode 510, and the negative electrode lead electrode 511. The exterior body 509 is sealed in a region 514.

The laminated secondary battery 600 can be manufactured using a manufacturing apparatus illustrated in FIG. 14, for example. A manufacturing apparatus 570 illustrated in FIG. 14 includes a component introduction chamber 571, a transfer chamber 572, a processing chamber 573, and a component extraction chamber 576. A structure can be employed in which each chamber is connected to a variety of exhaust mechanisms depending on usage. Alternatively, a structure can be employed in which each chamber is connected to a variety of gas supply mechanisms depending on usage. An inert gas is preferably supplied into the manufacturing apparatus 570 to inhibit entry of impurities into the manufacturing apparatus 570. Note that a gas that has been highly purified by a gas purifier before introduction into the manufacturing apparatus 570 is preferably used as the gas supplied into the manufacturing apparatus 570. The component introduction chamber 571 is a chamber for introducing the positive electrode, the separator, the negative electrode, the exterior body, and the like into the chambers such as the transfer chamber 572 and the processing chamber 573 in the manufacturing apparatus 570. The transfer chamber 572 includes a transfer mechanism 580. The treatment chamber 573 includes a stage and an electrolyte dripping mechanism. The component extraction chamber 576 is a chamber for extracting the manufactured secondary battery to the outside of the manufacturing apparatus 570.

A procedure for manufacturing the laminated secondary battery 600 is as follows.

First, an exterior body 509b is placed over a stage 591 in the treatment chamber 573, a frame-like resin layer 513 is formed over the exterior body 509b, and then the positive electrode 503 is placed over the exterior body 509b (FIG. 16A and FIG. 16B). Next, an electrolyte 515a is dripped on the positive electrode 503 from a nozzle 594 (FIG. 16C and FIG. 16D). FIG. 16D is a cross-sectional view taken along the dashed-dotted line A-B in FIG. 16C. Note that to avoid complexity of the diagram, the stage 591 is not illustrated in some cases. As a dripping method, any one of a dispensing method, a spraying method, an inkjet method, and the like can be used, for example. In addition, an ODF (One Drop Fill) method can be used for dripping the electrolyte.

With movement of the nozzle 594, the electrolyte 515a can be dripped on the entire surface of the positive electrode 503. Alternatively, with movement of the stage 591, the electrolyte 515a may be dripped on the entire surface of the positive electrode 503.

It is preferable to drip the electrolyte from a position whose shortest distance from a surface where the electrolyte is dripped is greater than 0 mm and less than or equal to 1 mm.

The viscosity of the electrolyte dripped from the nozzle or the like is preferably adjusted as appropriate. When the viscosity of the whole electrolyte falls within the range from 0.3 mPa·s to 1000 mPa·s at room temperature (25° C.), the electrolyte can be dripped from the nozzle.

Since the viscosity of the electrolyte changes depending on the temperature of the electrolyte, the temperature of the electrolyte to be dripped is preferably adjusted as appropriate. The temperature of the electrolyte is preferably higher than or equal to the melting point and lower than or equal to the boiling point and flash point of the electrolyte.

Then, the separator 507 is placed over the positive electrode 503 to overlap with the entire surface of the positive electrode 503 (FIG. 17A). Next, an electrolyte 515b is dripped on the separator 507 using the nozzle 594 (FIG. 17B). Then, the negative electrode 506 is placed over the separator 507 (FIG. 17C). The negative electrode 506 is placed to overlap with the separator 507 so that it does not protrude from the separator 507 in a top view. Next, an electrolyte 515c is dripped on the negative electrode 506 using the nozzle 594 (FIG. 17D). After that, the stacks including the positive electrodes 503, the separators 507, and the negative electrodes 506 are further stacked, so that a stack 512 illustrated in FIG. 15 can be fabricated. Next, the positive electrodes 503, the separators 507, and the negative electrodes 506 are sealed with an exterior body 509a and the exterior body 509b (FIG. 17E and FIG. 17F).

In FIG. 15, the positive electrode and the negative electrode are placed so that the separator is sandwiched between the positive electrode active material layer and the negative electrode active material layer. Note that in the secondary battery of one embodiment of the present invention, a region where the positive electrode active material layer and the negative electrode active material layer do not face each other is preferably small or not provided. In the case where the electrolyte contains an ionic liquid and a region where the negative electrode active material layer and the positive electrode active material layer do not face each other is provided, the charge and discharge efficiency of the secondary battery might decrease. Thus, in the secondary battery of one embodiment of the present invention, an end portion of the positive electrode active material layer and an end portion of the negative electrode active material layer are preferably aligned with each other to the utmost, for example. Therefore, the areas of the positive electrode active material layer and the negative electrode active material layer are preferably equal to each other when seen from above. Alternatively, the end portion of the positive electrode active material layer is preferably located inward from the end portion of the negative electrode active material layer.

Multiple formation can be performed by placing a plurality of stacks 512 on the exterior body 509b. The stacks 512 are each sealed with the exterior bodies 509a and 509b in the region 514 so that the active material layers are surrounded, and then the stacks 512 are divided outside the regions 514, whereby a plurality of secondary batteries can be individually separated.

In sealing, first, the frame-like resin layer 513 is formed over the exterior body 509b. Then, at least part of the resin layer 513 is irradiated with light under reduced pressure, so that at least part of the resin layer 513 is cured. Next, the sealing is performed in the region 514 by thermocompression bonding or welding under atmospheric pressure. Alternatively, it is possible that the sealing by light irradiation is not performed and only the sealing by thermocompression bonding or welding is performed.

Although FIG. 13 shows an example in which four sides of the exterior body 509 are sealed (referred to as four-side sealing in some cases), three sides may be sealed (referred to as three-side sealing in some cases) as illustrated in FIG. 10A and FIG. 10B.

Through the above process, the laminated secondary battery 600 can be manufactured.

### <Another Secondary Battery 1 and Manufacturing Method Thereof>

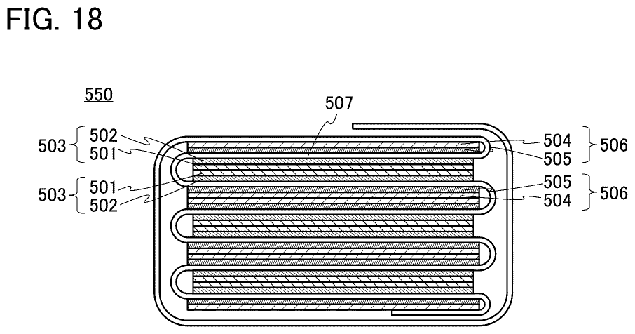

FIG. 18 shows an example of a cross-sectional view of a stack of one embodiment of the present invention. A stack 550 illustrated in FIG. 18 is fabricated by placing one folded separator between the positive electrode and the negative electrode.

In the stack 550, one separator 507 is folded a plurality of times to be sandwiched between the positive electrode active material layer 502 and the negative electrode active material layer 505. Since six positive electrodes 503 and six negative electrodes 506 are stacked in FIG. 18, the separator 507 is folded at least five times. The separator 507 is provided to be sandwiched between the positive electrode active material layer 502 and the negative electrode active material layer 505 and to have an extending portion folded such that the plurality of positive electrodes 503 and the plurality of negative electrodes 506 may be bound together with a tape or the like.

After the positive electrode 503 is placed, an electrolyte can be dripped on the positive electrode 503 in the method for manufacturing the secondary battery of one embodiment of the present invention. Similarly, after the negative electrode 506 is placed, an electrolyte can be dripped on the negative electrode 506. In the method for manufacturing the secondary battery of one embodiment of the present invention, an electrolyte can be dripped on the separator 507 before the separator is folded or after the folded separator 507 overlaps with the negative electrode 506 or the positive electrode 503. When an electrolyte is dripped on at least one of the negative electrode 506, the separator 507, and the positive electrode 503, the negative electrode 506, the separator 507, or the positive electrode 503 can be impregnated with the electrolyte.

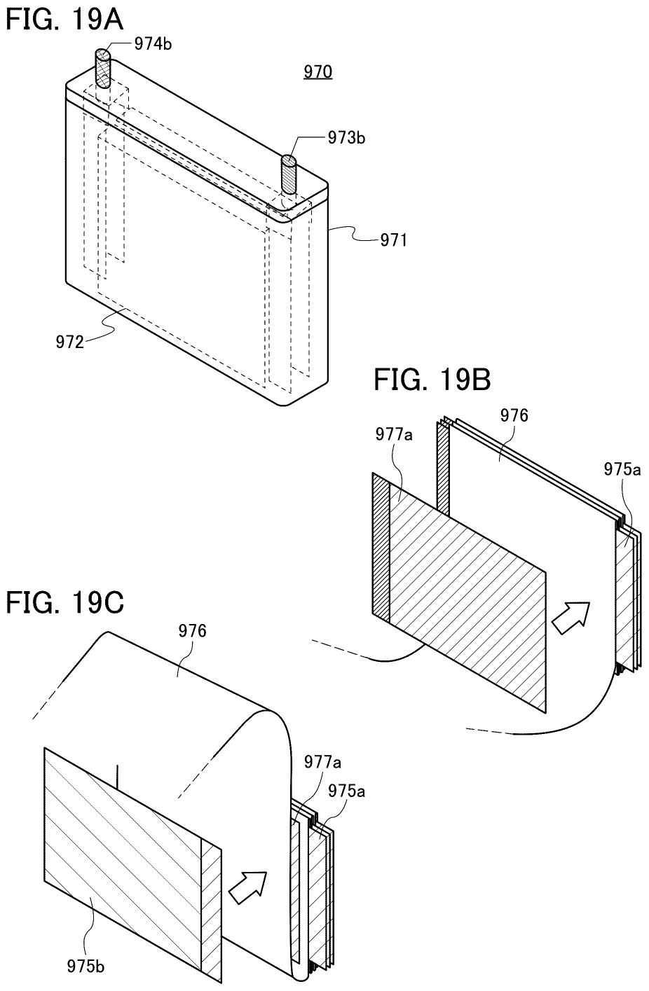

A secondary battery 970 illustrated in FIG. 19A includes a stack 972 inside a housing 971. A terminal 973b and a terminal 974b are electrically connected to the stack 972. At least part of the terminal 973b and at least part of the terminal 974b are exposed to the outside of the housing 971.

The stack 972 can have a stacked-layer structure of a positive electrode, a negative electrode, and a separator. Alternatively, the stack 972 can have a structure in which a positive electrode, a negative electrode, and a separator are wound, for example.

As the stack 972, the stack having the structure illustrated in FIG. 18 in which the separator is folded can be used, for example.

An example of a method for fabricating the stack 972 will be described with reference to FIG. 19B and FIG. 19C.

First, as illustrated in FIG. 19B, a belt-like separator 976 overlaps with a positive electrode 975a, and a negative electrode 977a overlaps with the positive electrode 975a with the separator 976 therebetween. After that, the separator 976 is folded to overlap with the negative electrode 977a. Next, as illustrated in FIG. 19C, a positive electrode 975b overlaps with the negative electrode 977a with the separator 976 therebetween. In this manner, the positive electrodes and the negative electrodes are sequentially placed with the folded separator therebetween, whereby the stack 972 can be fabricated. A structure including the stack fabricated in the above manner is sometimes referred to as a “zigzag structure”.

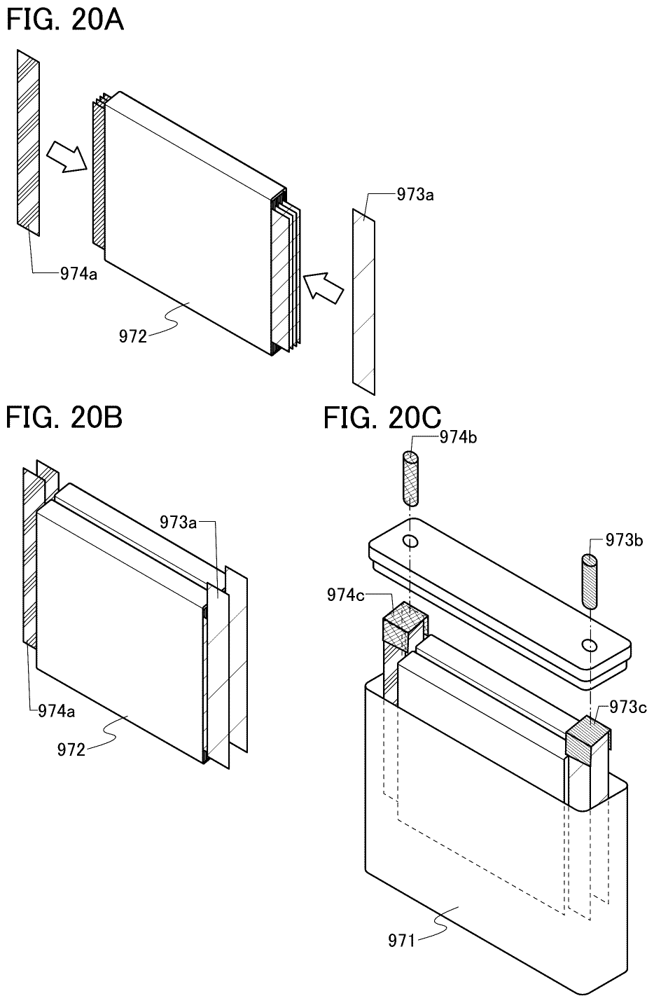

Next, an example of a method for manufacturing the secondary battery 970 will be described with reference to FIG. 20A to FIG. 20C.

First, as illustrated in FIG. 20A, a positive electrode lead electrode 973a is electrically connected to the positive electrodes included in the stack 972. Specifically, for example, the positive electrodes included in the stack 972 are provided with tab regions, and the tab regions and the positive electrode lead electrode 973a can be electrically connected to each other by welding or the like. In addition, a negative electrode lead electrode 974a is electrically connected to the negative electrodes included in the stack 972.

One stack 972 may be placed inside the housing 971 or a plurality of stacks 972 may be placed inside the housing 971. FIG. 20B shows an example of preparing two stacks 972.

Next, as illustrated in FIG. 20C, the prepared stacks 972 are stored in the housing 971, and the terminal 973b and the terminal 974b are inserted to seal the housing 971. It is preferable to electrically connect a conductor 973c to each of the positive electrode lead electrodes 973a included in the plurality of stacks 972. In addition, it is preferable to electrically connect a conductor 974c to each of the negative electrode lead electrodes 974a included in the plurality of stacks 972. The terminal 973b and the terminal 974b are electrically connected to the conductor 973c and the conductor 974c, respectively. Note that the conductor 973c may include a conductive region and an insulating region. In addition, the conductor 974c may include a conductive region and an insulating region.

For the housing 971, a metal material (e.g., aluminum) can be used. In the case where a metal material is used for the housing 971, the surface is preferably coated with a resin or the like. Alternatively, a resin material can be used for the housing 971.

The housing 971 is preferably provided with a safety valve, an overcurrent protection element, or the like. A safety valve is a valve for releasing a gas, in order to prevent the battery from exploding, when the pressure inside the housing 971 reaches a predetermined pressure.

### <Another Secondary Battery 2 and Manufacturing Method Thereof>

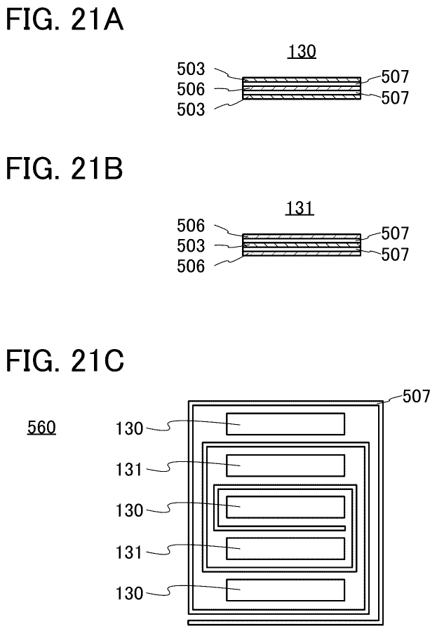

FIG. 21C shows an example of a cross-sectional view of a secondary battery of another embodiment of the present invention. A secondary battery 560 illustrated in FIG. 21C is manufactured using stacks 130 illustrated in FIG. 21A and stacks 131 illustrated in FIG. 21B. In FIG. 21C, the stacks 130, the stacks 131, and the separator 507 are selectively illustrated for the sake of clarity of the drawing.

As illustrated in FIG. 21A, in the stack 130, the positive electrode 503 including the positive electrode active material layers on both surfaces of the positive electrode current collector, the separator 507, the negative electrode 506 including the negative electrode active material layers on both surfaces of the negative electrode current collector, the separator 507, and the positive electrode 503 including the positive electrode active material layers on both surfaces of the positive electrode current collector are stacked in this order.

As illustrated in FIG. 21B, in the stack 131, the negative electrode 506 including the negative electrode active material layers on both surfaces of the negative electrode current collector, the separator 507, the positive electrode 503 including the positive electrode active material layers on both surfaces of the positive electrode current collector, the separator 507, and the negative electrode 506 including the negative electrode active material layers on both surfaces of the negative electrode current collector are stacked in this order.

The method for manufacturing the secondary battery of one embodiment of the present invention can be utilized for fabricating the stacks. Specifically, in order to fabricate the stacks, an electrolyte is dripped on at least one of the negative electrode 506, the separator 507, and the positive electrode 503 at the time of stacking the negative electrode 506, the separator 507, and the positive electrode 503. Dripping a plurality of drops of the electrolyte enables the negative electrode 506, the separator 507, or the positive electrode 503 to be impregnated with the electrolyte.

As illustrated in FIG. 21C, the plurality of stacks 130 and the plurality of stacks 131 are covered with the wound separator 507.

After the stacks 130 are placed, an electrolyte can be dripped on the stacks 130 in the method for manufacturing the secondary battery of one embodiment of the present invention. Similarly, after the stacks 131 are placed, an electrolyte can be dripped on the stacks 131. Moreover, an electrolyte can be dripped on the separator 507 before the separator 507 is folded or after the folded separator 507 overlaps with the stacks. Dripping a plurality of drops of the electrolyte enables the stacks 130, the stacks 131, or the separator 507 to be impregnated with the electrolyte.

### <Another Secondary Battery 3 and Manufacturing Method Thereof>

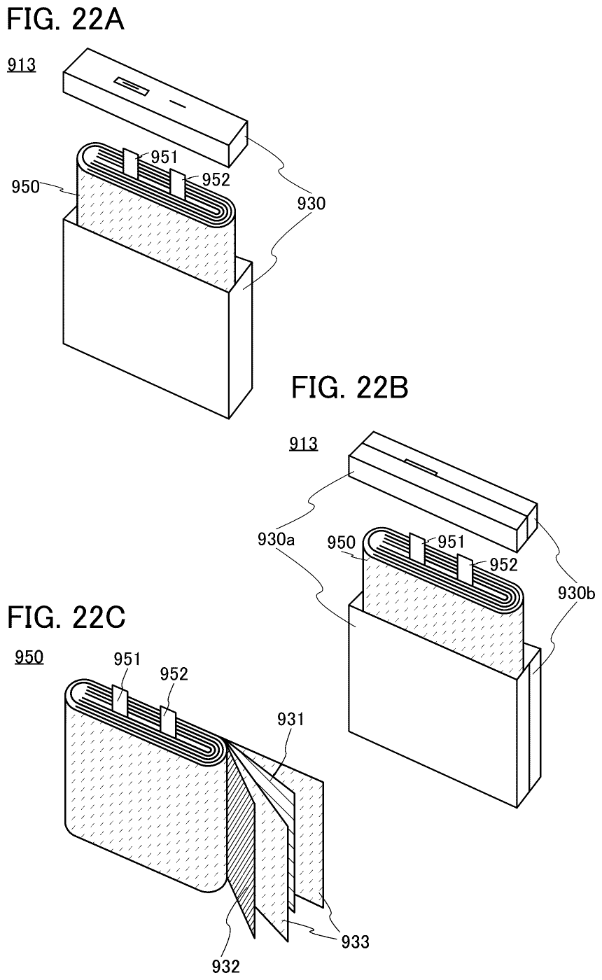

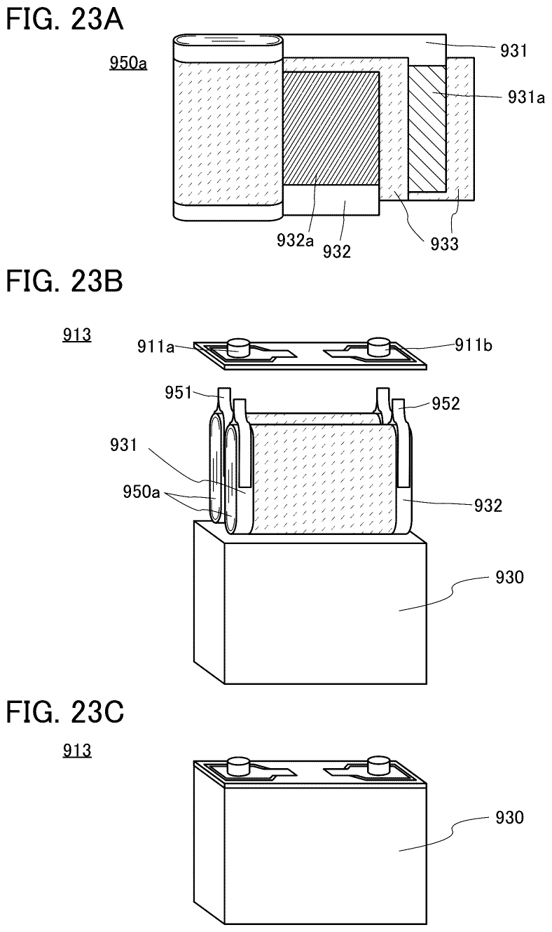

A secondary battery of another embodiment of the present invention will be described with reference to FIG. 22 and FIG. 23. The secondary battery described here can be referred to as a wound secondary battery or the like.

A secondary battery 913 illustrated in FIG. 22A includes a wound body 950 provided with a terminal 951 and a terminal 952 inside a housing 930. The wound body 950 is immersed in an electrolyte inside the housing 930. The terminal 952 is in contact with the housing 930. The use of an insulator or the like inhibits contact between the terminal 951 and the housing 930. Note that in FIG. 22A, the housing 930 divided into pieces is illustrated for convenience; however, in the actual structure, the wound body 950 is covered with the housing 930 and the terminal 951 and the terminal 952 extend to the outside of the housing 930. For the housing 930, a metal material (e.g., aluminum) or a resin material can be used.

Note that as illustrated in FIG. 22B, the housing 930 illustrated in FIG. 22A may be formed using a plurality of materials. For example, in the secondary battery 913 illustrated in FIG. 22B, a housing 930a and a housing 930b are bonded to each other, and the wound body 950 is provided in a region surrounded by the housing 930a and the housing 930b.

For the housing 930a, an insulating material such as an organic resin can be used. In particular, when a material such as an organic resin is used for the side on which an antenna is formed, blocking of an electric field by the secondary battery 913 can be inhibited. When an electric field is not significantly blocked by the housing 930a, an antenna may be provided inside the housing 930a. For the housing 930b, a metal material can be used, for example.

Furthermore, FIG. 22C illustrates the structure of the wound body 950. The wound body 950 includes a negative electrode 931, a positive electrode 932, and separators 933. The wound body 950 is a wound body obtained by winding a sheet of a stack in which the negative electrode 931 and the positive electrode 932 overlap with each other with the separator 933 therebetween. Note that a plurality of stacked layers each including the negative electrode 931, the positive electrode 932, and the separators 933 may be further stacked.

At the time of stacking the negative electrode 931, the separator 933, and the positive electrode 932 in the method for manufacturing the secondary battery of one embodiment of the present invention, an electrolyte is dripped on at least one of the negative electrode 931, the separator 933, and the positive electrode 932. That is, an electrolyte is preferably dripped before the sheet of the stack is wound. Dripping a plurality of drops of the electrolyte enables the negative electrode 931, the separator 933, or the positive electrode 932 to be impregnated with the electrolyte.

As illustrated in FIG. 23A, the secondary battery 913 may include a wound body 950a. The wound body 950a illustrated in FIG. 23A includes the negative electrode 931, the positive electrode 932, and the separators 933. The negative electrode 931 includes a negative electrode active material layer 931a. The positive electrode 932 includes a positive electrode active material layer 932a.

The separator 933 has a larger width than the negative electrode active material layer 931a and the positive electrode active material layer 932a, and is wound to overlap with the negative electrode active material layer 931a and the positive electrode active material layer 932a. In terms of safety, the width of the negative electrode active material layer 931a is preferably larger than that of the positive electrode active material layer 932a. The wound body 950a having such a shape is preferable because of its high level of safety and high productivity.

As illustrated in FIG. 23B, the negative electrode 931 is electrically connected to the terminal 951. The terminal 951 is electrically connected to a terminal 911a. The positive electrode 932 is electrically connected to the terminal 952. The terminal 952 is electrically connected to a terminal 911b.

As illustrated in FIG. 23C, the wound body 950a and an electrolyte are covered with the housing 930, whereby the secondary battery 913 is completed. The housing 930 is preferably provided with a safety valve, an overcurrent protection element, and the like. In order to prevent the battery from exploding, a safety valve is temporarily released when the internal pressure of the housing 930 exceeds a predetermined internal pressure.

As illustrated in FIG. 23B, the secondary battery 913 may include a plurality of wound bodies 950a. The use of the plurality of wound bodies 950a enables the secondary battery 913 to have higher charge and discharge capacity.

This embodiment can be combined with the other embodiments as appropriate.

### Embodiment 4

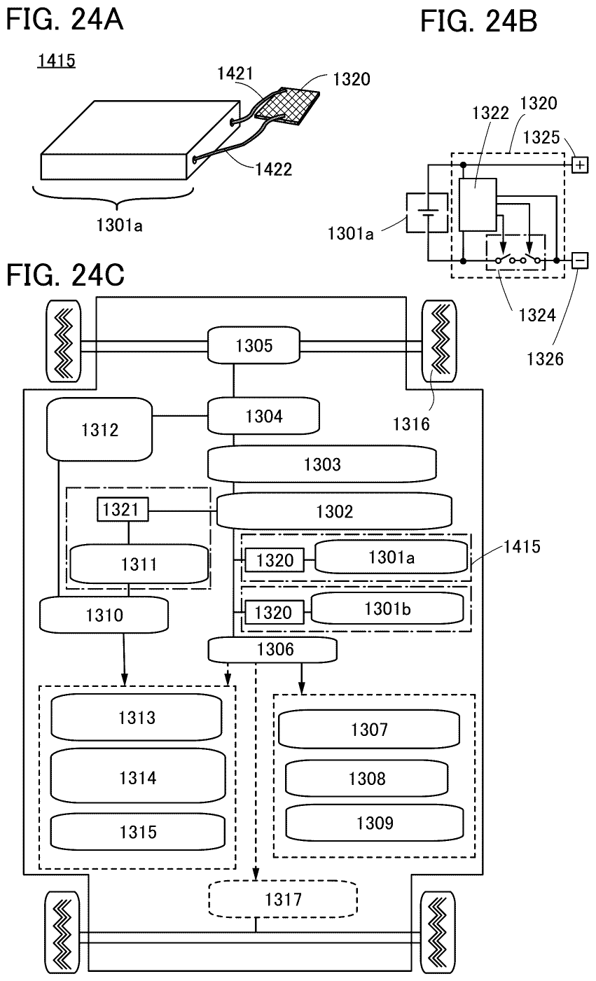

In this embodiment, application examples of the secondary battery of one embodiment of the present invention will be described with reference to FIG. 24 to FIG. 33.

### [Vehicle]

First, an example in which the secondary battery of one embodiment of the present invention is used in an electric vehicle (EV) will be described.

FIG. 24C shows a block diagram of a vehicle including a motor. The electric vehicle is provided with first batteries 1301a and 1301b as main secondary batteries for driving and a second battery 1311 that supplies electric power to an inverter 1312 for starting a motor 1304. The second battery 1311 is also referred to as a cranking battery or a starter battery. The second battery 1311 only needs high output and high capacity is not so much needed; the capacity of the second battery 1311 is lower than that of the first batteries 1301a and 1301b.

For example, as one or both of the first batteries 1301a and 1301b, the secondary battery manufactured by the method for manufacturing the secondary battery of one embodiment of the present invention can be used.

Although this embodiment shows an example in which the two first batteries 1301a and 1301b are connected in parallel, three or more batteries may be connected in parallel. In the case where the first battery 1301a can store sufficient electric power, the first battery 1301b may be omitted. With a battery pack including a plurality of secondary batteries, large electric power can be extracted. The plurality of secondary batteries may be connected in parallel, connected in series, or connected in series after being connected in parallel. The plurality of secondary batteries are also referred to as an assembled battery.

An in-vehicle secondary battery includes a service plug or a circuit breaker that can cut off high voltage without the use of equipment in order to cut off electric power from a plurality of secondary batteries. The first battery 1301a is provided with such a service plug or a circuit breaker.

Electric power from the first batteries 1301a and 1301b is mainly used to rotate the motor 1304 and is also supplied to in-vehicle parts for 42 V (for a high-voltage system) (such as an electric power steering 1307, a heater 1308, and a defogger 1309) through a DCDC circuit 1306. In the case where there is a rear motor 1317 for the rear wheels, the first battery 1301a is used to rotate the rear motor 1317.

The second battery 1311 supplies electric power to in-vehicle parts for 14 V (for a low-voltage system) (such as an audio 1313, power windows 1314, and lamps 1315) through a DCDC circuit 1310.

The first battery 1301a will be described with reference to FIG. 24A.

FIG. 24A shows an example of a large battery pack 1415. One electrode of the battery pack 1415 is electrically connected to a control circuit portion 1320 through a wiring 1421. The other electrode is electrically connected to the control circuit portion 1320 through a wiring 1422. Note that the battery pack may have a structure in which a plurality of secondary batteries are connected in series.

The control circuit portion 1320 may include a memory circuit including a transistor using an oxide semiconductor. A charging control circuit or a battery control system that includes a memory circuit including a transistor using an oxide semiconductor may be referred to as a BTOS (Battery operating system or Battery oxide semiconductor).

The control circuit portion 1320 senses a terminal voltage of the secondary battery and controls the charging and discharging state of the secondary battery. For example, to prevent overcharging, an output transistor of a charging circuit and an interruption switch can be turned off substantially at the same time.

FIG. 24B shows an example of a block diagram of the battery pack 1415 illustrated in FIG. 24A.

The control circuit portion 1320 includes a switch portion 1324 that includes at least a switch for preventing overcharging and a switch for preventing overdischarging, a control circuit 1322 for controlling the switch portion 1324, and a portion for measuring the voltage of the first battery 1301a. The control circuit portion 1320 is set to have the upper limit voltage and the lower limit voltage of the secondary battery to be used, and imposes the upper limit of current from the outside, the upper limit of output current to the outside, or the like. The range from the lower limit voltage to the upper limit voltage of the secondary battery is a recommended voltage range, and when a voltage falls outside the range, the switch portion 1324 operates and functions as a protection circuit. The control circuit portion 1320 can also be referred to as a protection circuit because it controls the switch portion 1324 to prevent overdischarging or overcharging. For example, when the control circuit 1322 detects a voltage that is likely to cause overcharging, current is interrupted by turning off the switch in the switch portion 1324. Furthermore, a function of interrupting current in accordance with a temperature rise may be set by providing a PTC element in the charging and discharging path. The control circuit portion 1320 includes an external terminal 1325 (+IN) and an external terminal 1326 (−IN).

The switch portion 1324 can be formed by a combination of n-channel transistors and/or p-channel transistors. The switch portion 1324 is not limited to a switch including a Si transistor using single crystal silicon; the switch portion 1324 may be formed using a power transistor containing Ge (germanium), SiGe (silicon germanium), GaAs (gallium arsenide), GaAlAs (gallium aluminum arsenide), InP (indium phosphide), SiC (silicon carbide), ZnSe (zinc selenide), GaN (gallium nitride), GaO′ (gallium oxide, where x is a real number greater than 0), or the like. A memory element using an OS transistor can be freely placed by being stacked over a circuit using a Si transistor, for example; hence, integration can be easy. Furthermore, an OS transistor can be fabricated with a manufacturing apparatus similar to that for a Si transistor and thus can be fabricated at low cost. That is, the control circuit portion 1320 using OS transistors can be stacked over the switch portion 1324 so that they can be integrated into one chip. Since the volume occupied by the control circuit portion 1320 can be reduced, a reduction in size is possible.

The first batteries 1301a and 1301b mainly supply electric power to in-vehicle parts for 42 V (for a high-voltage system), and the second battery 1311 supplies electric power to in-vehicle parts for 14 V (for a low-voltage system). A lead storage battery is usually used for the second battery 1311 due to cost advantage.

In this embodiment, an example in which a lithium-ion secondary battery is used as both the first battery 1301a and the second battery 1311 is described. As the second battery 1311, a lead storage battery, an all-solid-state battery, or an electric double layer capacitor may be used.

Regenerative energy generated by rolling of tires 1316 is transmitted to the motor 1304 through a gear 1305, and is stored in the second battery 1311 from a motor controller 1303 or a battery controller 1302 through a control circuit portion 1321. Alternatively, the regenerative energy is stored in the first battery 1301a from the battery controller 1302 through the control circuit portion 1320. Alternatively, the regenerative energy is stored in the first battery 1301b from the battery controller 1302 through the control circuit portion 1320. For efficient charging with regenerative energy, the first batteries 1301a and 1301b are desirably capable of fast charging.

The battery controller 1302 can set the charge voltage, charge current, and the like of the first batteries 1301a and 1301b. The battery controller 1302 can set charging conditions in accordance with charging characteristics of a secondary battery to be used, so that fast charging can be performed.

Although not illustrated, in the case of connection to an external charger, a plug of the charger or a connection cable of the charger is electrically connected to the battery controller 1302. Electric power supplied from the external charger is stored in the first batteries 1301a and 1301b through the battery controller 1302. Some chargers are provided with a control circuit, in which case the function of the battery controller 1302 is not used; to prevent overcharging, the first batteries 1301a and 1301b are preferably charged through the control circuit portion 1320. In addition, a connection cable or a connection cable of the charger is sometimes provided with a control circuit. The control circuit portion 1320 is also referred to as an ECU (Electronic Control Unit). The ECU is connected to a CAN (Controller Area Network) provided in the electric vehicle. The CAN is a type of a serial communication standard used as an in-vehicle LAN. The ECU includes a microcomputer. Moreover, the ECU uses a CPU or a GPU.

Next, examples in which the secondary battery of one embodiment of the present invention is mounted on a vehicle, typically a transport vehicle, will be described.

By mounting the secondary battery of one embodiment of the present invention on vehicles, next-generation clean energy vehicles such as hybrid vehicles (HVs), electric vehicles (EVs), and plug-in hybrid vehicles (PHVs) can be achieved. The secondary battery can also be mounted on transport vehicles such as agricultural machines such as electric tractors, motorized bicycles including motor-assisted bicycles, motorcycles, electric wheelchairs, electric carts, boats or ships, submarines, aircraft such as fixed-wing aircraft and rotary-wing aircraft, rockets, artificial satellites, space probes, planetary probes, and spacecraft. With the use of the method for manufacturing the secondary battery of one embodiment of the present invention, a large secondary battery can be provided. Thus, the secondary battery of one embodiment of the present invention can be suitably used in transport vehicles.

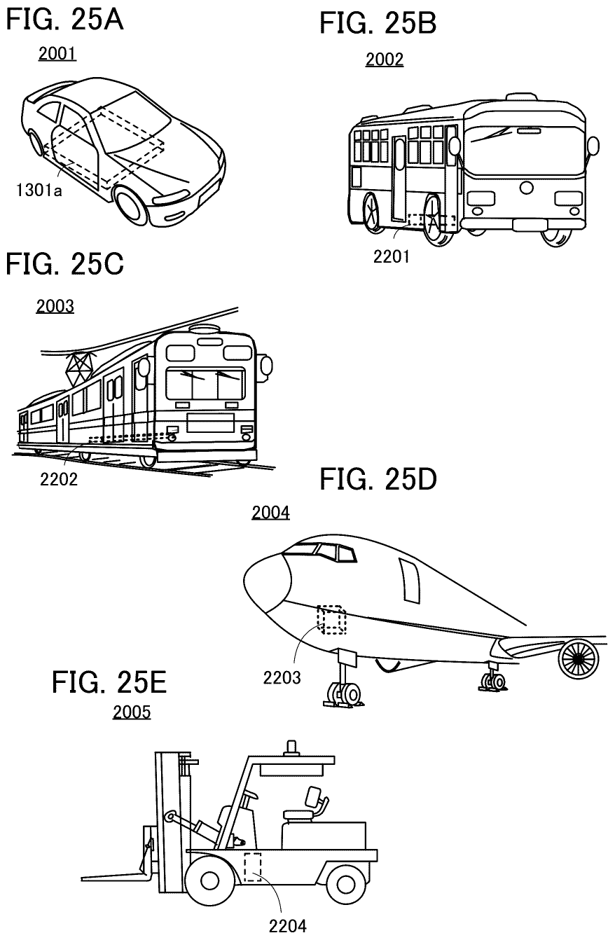

FIG. 25A to FIG. 25E illustrate transport vehicles each using the secondary battery of one embodiment of the present invention. A motor vehicle 2001 illustrated in FIG. 25A is an electric vehicle that runs using an electric motor as a driving power source. Alternatively, the motor vehicle 2001 is a hybrid vehicle that can appropriately select an electric motor or an engine as a driving power source. In the case where the secondary battery is mounted on the vehicle, the secondary battery is provided at one position or several positions. The motor vehicle 2001 illustrated in FIG. 25A includes the battery pack 1415 illustrated in FIG. 24A. The battery pack 1415 includes a secondary battery module. The battery pack 1415 preferably further includes a charging control device that is electrically connected to the secondary battery module. The secondary battery module includes one or more secondary batteries.

The motor vehicle 2001 can be charged when the secondary battery included in the motor vehicle 2001 is supplied with electric power through external charging equipment by a plug-in system, a contactless power feeding system, or the like. In charging, a given method such as CHAdeMO (registered trademark) or Combined Charging System can be employed as a charging method, the standard of a connector, or the like as appropriate. A charging device may be a charging station provided in a commerce facility or a power source in a house. For example, with the use of the plug-in technique, a secondary battery mounted on the motor vehicle 2001 can be charged by being supplied with electric power from the outside. The charging can be performed by converting AC electric power into DC electric power through a converter such as an ACDC converter.

Although not illustrated, the vehicle may include a power receiving device so that it can be charged by being supplied with electric power from an above-ground power transmitting device in a contactless manner. In the case of the contactless power feeding system, by fitting a power transmitting device in a road or an exterior wall, charging can be performed not only when the vehicle is stopped but also when driven. In addition, the contactless power feeding system may be utilized to perform transmission and reception of electric power between two vehicles. Furthermore, a solar cell may be provided in the exterior of the vehicle to charge the secondary battery when the vehicle stops or moves. To supply electric power in such a contactless manner, an electromagnetic induction method or a magnetic resonance method can be used.

FIG. 25B illustrates a large transporter 2002 having a motor controlled by electricity as an example of a transport vehicle. A secondary battery module of the transporter 2002 includes a cell unit of four secondary batteries with 3.5 V or higher and 4.7 V or lower, for example, and 48 cells are connected in series to have a maximum voltage of 170 V. A battery pack 2201 has the same function as the battery pack in FIG. 25A except, for example, the number of secondary batteries configuring the secondary battery module; thus, the description is omitted.

FIG. 25C illustrates a large transport vehicle 2003 having a motor controlled by electricity as an example. The secondary battery module of the transport vehicle 2003 has 100 or more secondary batteries with 3.5 V or higher and 4.7 V or lower connected in series, and the maximum voltage is 600 V, for example. Thus, the secondary batteries are required to have a small variation in the characteristics. With the use of the method for manufacturing the secondary battery of one embodiment of the present invention, a secondary battery with stable battery performance can be manufactured, and mass production at low cost is possible in view of the yield. A battery pack 2202 has the same function as the battery pack in FIG. 25A except, for example, the number of secondary batteries configuring the secondary battery module; thus, the description is omitted.

FIG. 25D illustrates an aircraft 2004 having a combustion engine as an example. The aircraft 2004 illustrated in FIG. 25D is regarded as a kind of transport vehicles because it has wheels for takeoff and landing, and includes a battery pack 2203 that includes a charging control device and a secondary battery module configured by connecting a plurality of secondary batteries.

The secondary battery module of the aircraft 2004 has eight 4 V secondary batteries connected in series and has a maximum voltage of 32 V, for example. The battery pack 2203 has the same function as the battery pack in FIG. 25A except, for example, the number of secondary batteries configuring the secondary battery module; thus, the description is omitted.

FIG. 25E illustrates a transport vehicle 2005 that transports a load as an example. The transport vehicle 2005 includes a motor controlled by electricity and executes various operations with use of electric power supplied from secondary batteries configuring a secondary battery module of a battery pack 2204. The transport vehicle 2005 is not limited to be operated by a human who rides thereon as a driver, and an unmanned operation is also possible by CAN communication or the like. Although FIG. 25E illustrates a fork lift, there is no particular limitation and a battery pack including the secondary battery of one embodiment of the present invention can be mounted on industrial machines capable of being operated by CAN communication or the like, e.g., automatic transporters, working robots, and small construction equipment.

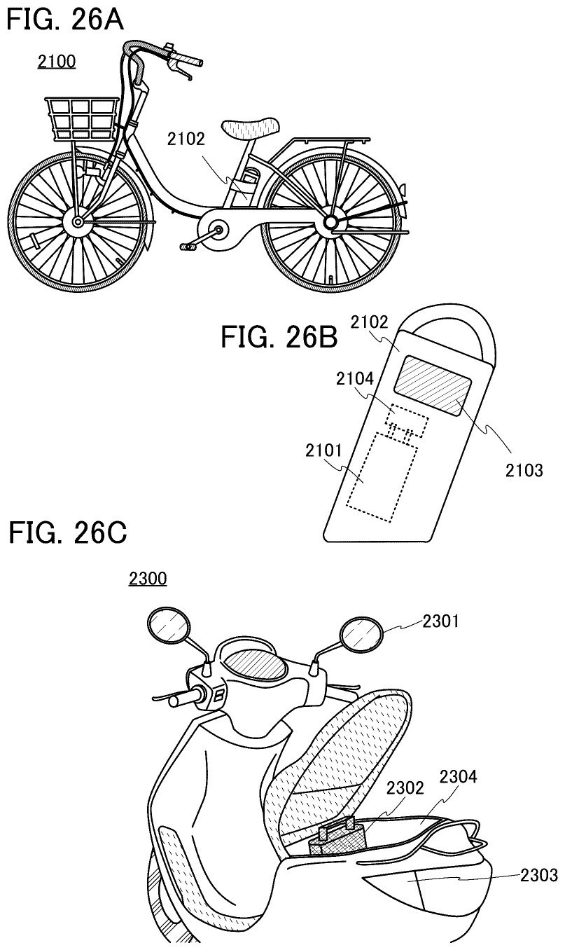

FIG. 26A shows an example of an electric bicycle using the secondary battery of one embodiment of the present invention. The secondary battery of one embodiment of the present invention can be used for an electric bicycle 2100 illustrated in FIG. 26A. A power storage device 2102 illustrated in FIG. 26B includes a plurality of secondary batteries and a protection circuit, for example.

The electric bicycle 2100 includes the power storage device 2102. The power storage device 2102 can supply electricity to a motor that assists a rider. The power storage device 2102 is portable, and FIG. 26B illustrates the state where the power storage device 2102 is detached from the bicycle. A plurality of secondary batteries 2101 of embodiments of the present invention are incorporated in the power storage device 2102, and the remaining battery capacity and the like can be displayed on a display portion 2103. The power storage device 2102 includes a control circuit 2104 capable of charging control or anomaly detection for the secondary battery, which is exemplified in one embodiment of the present invention. The control circuit 2104 is electrically connected to a positive electrode and a negative electrode of the secondary battery 2101. The control circuit 2104 may be provided with a small solid-state secondary battery. When the small solid-state secondary battery is provided in the control circuit 2104, electric power can be supplied to retain data in a memory circuit included in the control circuit 2104 for a long time. When the control circuit 2104 is used in combination with the secondary battery including the positive electrode active material 100 of one embodiment of the present invention in the positive electrode, the synergy on safety can be obtained. The secondary battery including the positive electrode active material 100 of one embodiment of the present invention in the positive electrode and the control circuit 2104 can greatly contribute to elimination of accidents due to secondary batteries, such as fires.

FIG. 26C shows an example of a motorcycle including the secondary battery of one embodiment of the present invention. A motor scooter 2300 illustrated in FIG. 26C includes a power storage device 2302, side mirrors 2301, and indicator lights 2303. The power storage device 2302 can supply electricity to the indicator lights 2303. The power storage device 2302 including a plurality of secondary batteries including a positive electrode using the positive electrode active material 100 of one embodiment of the present invention can have high capacity and contribute to a reduction in size. To improve safety, a protection circuit that prevents overcharging and/or overdischarging of the secondary battery may be electrically connected to the secondary battery.

In the motor scooter 2300 illustrated in FIG. 26C, the power storage device 2302 can be stored in an under-seat storage unit 2304. The power storage device 2302 can be stored in the under-seat storage unit 2304 even with a small size.

### [Building]

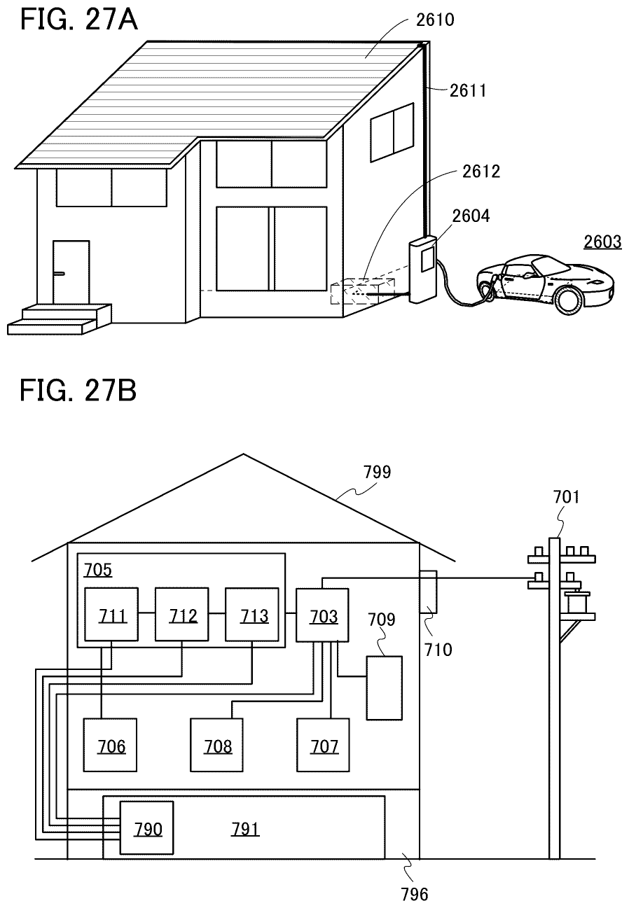

Next, examples in which the secondary battery of one embodiment of the present invention is mounted on a building will be described with reference to FIG. 27.

A house illustrated in FIG. 27A includes a power storage device 2612 including the secondary battery that has stable battery performance by employing the method for manufacturing the secondary battery of one embodiment of the present invention and a solar panel 2610. The power storage device 2612 is electrically connected to the solar panel 2610 through a wiring 2611 or the like. The power storage device 2612 may be electrically connected to a ground-based charging device 2604. The power storage device 2612 can be charged with electric power generated by the solar panel 2610. The secondary battery included in a vehicle 2603 can be charged with the electric power stored in the power storage device 2612 through the charging device 2604. The power storage device 2612 is preferably provided in an underfloor space. The power storage device 2612 is provided in the underfloor space, in which case the space on the floor can be effectively used. Alternatively, the power storage device 2612 may be provided on the floor.

The electric power stored in the power storage device 2612 can also be supplied to other electronic devices in the house. Thus, with the use of the power storage device 2612 as an uninterruptible power source, electronic devices can be used even when electric power cannot be supplied from a commercial power source due to power failure or the like.

FIG. 27B shows an example of a power storage device of one embodiment of the present invention. As illustrated in FIG. 27B, a large power storage device 791 including a secondary battery obtained by the method for manufacturing the secondary battery of one embodiment of the present invention is provided in an underfloor space 796 of a building 799.

The power storage device 791 is provided with a control device 790, and the control device 790 is electrically connected to a distribution board 703, a power storage controller 705 (also referred to as a control device), an indicator 706, and a router 709 through wirings.

Electric power is transmitted from a commercial power source 701 to the distribution board 703 through a service wire mounting portion 710. Moreover, electric power is transmitted to the distribution board 703 from the power storage device 791 and the commercial power source 701, and the distribution board 703 supplies the transmitted electric power to a general load 707 and a power storage load 708 through outlets (not illustrated).

The general load 707 is, for example, an electric device such as a TV or a personal computer. The power storage load 708 is, for example, an electric device such as a microwave oven, a refrigerator, or an air conditioner.

The power storage controller 705 includes a measuring portion 711, a predicting portion 712, and a planning portion 713. The measuring portion 711 has a function of measuring the amount of electric power consumed by the general load 707 and the power storage load 708 during a day (e.g., from midnight to midnight). The measuring portion 711 may have a function of measuring the amount of electric power of the power storage device 791 and the amount of electric power supplied from the commercial power source 701. The predicting portion 712 has a function of predicting, on the basis of the amount of electric power consumed by the general load 707 and the power storage load 708 during a given day, the demand for electric power consumed by the general load 707 and the power storage load 708 during the next day. The planning portion 713 has a function of making a charging and discharging plan of the power storage device 791 on the basis of the demand for electric power predicted by the predicting portion 712.

The amount of electric power consumed by the general load 707 and the power storage load 708 and measured by the measuring portion 711 can be checked with the indicator 706. It can be checked with an electric device such as a TV or a personal computer through the router 709. Furthermore, it can be checked with a portable electronic terminal such as a smartphone or a tablet through the router 709. With the indicator 706, the electric device, or the portable electronic terminal, for example, the demand for electric power depending on a time period (or per hour) that is predicted by the predicting portion 712 can be checked.

### [Electronic Device]

The secondary battery of one embodiment of the present invention can be used for one or both of an electronic device and a lighting device, for example. Examples of the electronic device include portable information terminals such as mobile phones, smartphones, and laptop computers; portable game machines; portable music players; digital cameras; and digital video cameras.

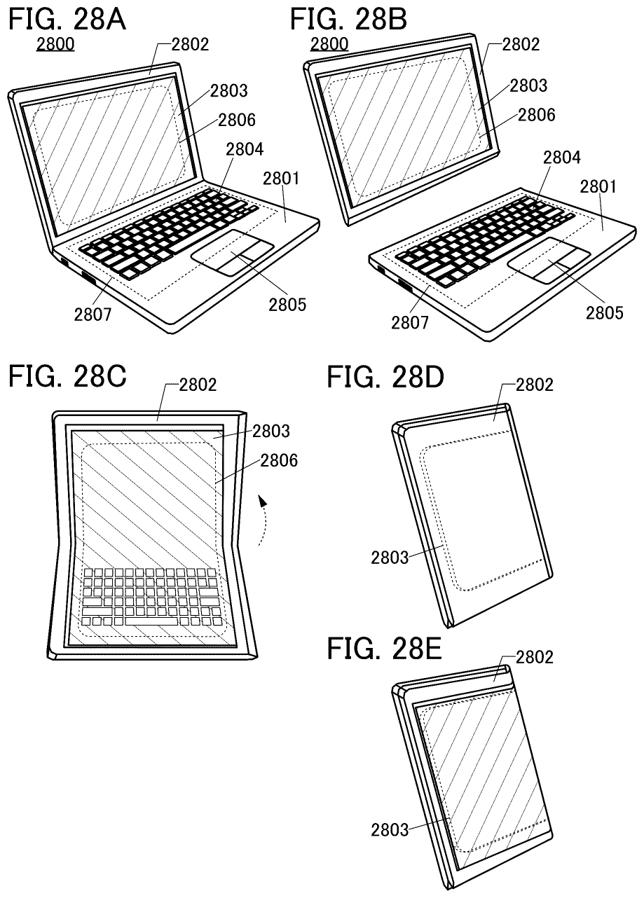

A personal computer 2800 illustrated in FIG. 28A includes a housing 2801, a housing 2802, a display portion 2803, a keyboard 2804, a pointing device 2805, and the like. A secondary battery 2807 is provided inside the housing 2801, and a secondary battery 2806 is provided inside the housing 2802. To improve safety, a protection circuit that prevents overcharging and/or overdischarging of the secondary batteries 2807 and 2806 may be electrically connected to the secondary batteries 2807 and 2806. A touch panel is used for the display portion 2803. As illustrated in FIG. 28B, the housing 2801 and the housing 2802 of the personal computer 2800 can be detached and the housing 2802 can be used alone as a tablet terminal.

The large secondary battery obtained by the method for manufacturing the secondary battery of one embodiment of the present invention can be used as one or both of the secondary battery 2806 and the secondary battery 2807. The shape of the secondary battery obtained by the method for manufacturing the secondary battery of one embodiment of the present invention can be changed freely by changing the shape of the exterior body. When the shapes of the secondary batteries 2806 and 2807 fit with the shapes of the housings 2801 and 2802, for example, the secondary batteries can have high capacity and thus the operating time of the personal computer 2800 can be lengthened. Moreover, the weight of the personal computer 2800 can be reduced.

A flexible display is used for the display portion 2803 of the housing 2802. As the secondary battery 2806, the large secondary battery obtained by the method for manufacturing the secondary battery of one embodiment of the present invention is used. With the use of a flexible film as the exterior body in the large secondary battery obtained by the method for manufacturing the secondary battery of one embodiment of the present invention, a bendable secondary battery can be obtained. Thus, as illustrated in FIG. 28C, the housing 2802 can be used while being bent. In that case, part of the display portion 2803 can be used as a keyboard as illustrated in FIG. 28C.

Furthermore, the housing 2802 can be folded such that the display portion 2803 is placed inward as illustrated in FIG. 28D, and the housing 2802 can be folded such that the display portion 2803 faces outward as illustrated in FIG. 28E.

A bendable secondary battery to which the secondary battery of one embodiment of the present invention is applied can be mounted on an electronic device and incorporated along a curved inside/outside wall surface of a house or a building or a curved interior/exterior surface of a motor vehicle.

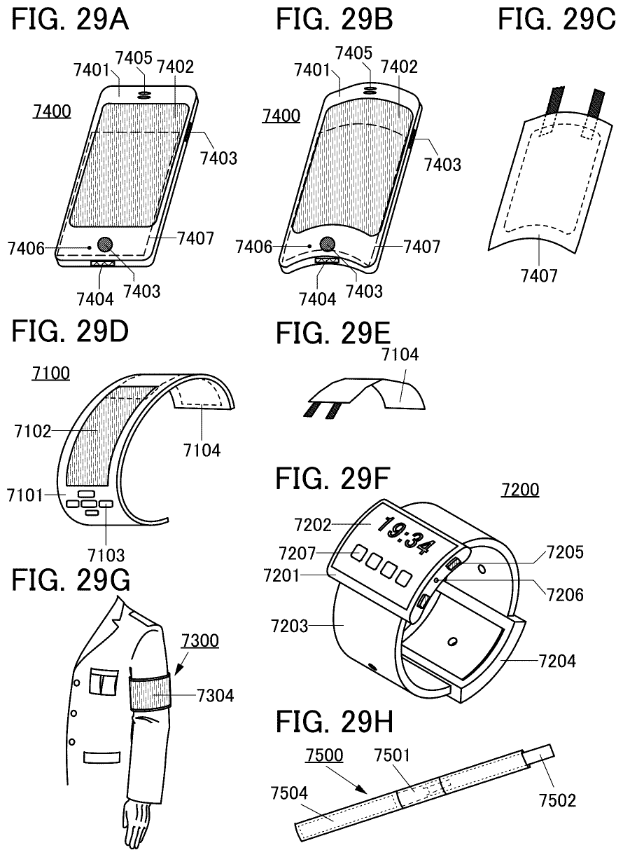

FIG. 29A shows an example of a mobile phone. A mobile phone 7400 is provided with a display portion 7402 incorporated in a housing 7401, operation buttons 7403, an external connection port 7404, a speaker 7405, a microphone 7406, and the like. Note that the mobile phone 7400 includes a secondary battery 7407. When the secondary battery of one embodiment of the present invention is used as the secondary battery 7407, a lightweight mobile phone with a long lifetime can be provided. To improve safety, a protection circuit that prevents overcharging and/or overdischarging of the secondary battery 7407 may be electrically connected to the secondary battery 7407.

FIG. 29B illustrates the mobile phone 7400 that is curved. When the whole mobile phone 7400 is curved by external force, the secondary battery 7407 provided therein is also curved. FIG. 29C illustrates the secondary battery 7407 that is being bent at that time. The secondary battery 7407 is a thin storage battery. The secondary battery 7407 is fixed in a state of being bent. Note that the secondary battery 7407 includes a lead electrode electrically connected to a current collector. The current collector is, for example, copper foil, and partly alloyed with gallium; thus, adhesion between the current collector and an active material layer in contact with the current collector is improved and the secondary battery 7407 can have high reliability even in a state of being bent.

FIG. 29D shows an example of a bangle display device. A portable display device 7100 includes a housing 7101, a display portion 7102, operation buttons 7103, and a secondary battery 7104. To improve safety, a protection circuit that prevents overcharging and/or overdischarging of the secondary battery 7104 may be electrically connected to the secondary battery 7104. FIG. 29E illustrates the bent secondary battery 7104. When the display device is worn on a user's arm while the secondary battery 7104 is bent, the housing changes its shape and the curvature of part or the whole of the secondary battery 7104 is changed. Note that the bending condition of a curve at a given point that is represented by a value of the radius of a corresponding circle is referred to as the radius of curvature, and the reciprocal of the radius of curvature is referred to as curvature. Specifically, part or the whole of the housing or the main surface of the secondary battery 7104 is changed in the range of radius of curvature from 40 mm or more to 150 mm or less. When the radius of curvature at the main surface of the secondary battery 7104 is in the range from 40 mm or more to 150 mm or less, the reliability can be kept high. When the secondary battery of one embodiment of the present invention is used as the secondary battery 7104, a lightweight portable display device with a long lifetime can be provided.

FIG. 29F shows an example of a watch-type portable information terminal. A portable information terminal 7200 includes a housing 7201, a display portion 7202, a band 7203, a buckle 7204, an operation button 7205, an input/output terminal 7206, and the like.

The portable information terminal 7200 is capable of executing a variety of applications such as mobile phone calls, e-mailing, viewing and editing texts, music reproduction, Internet communication, and a computer game.

The display surface of the display portion 7202 is curved, and images can be displayed on the curved display surface. In addition, the display portion 7202 includes a touch sensor, and operation can be performed by touching the screen with a finger, a stylus, or the like. For example, by touching an icon 7207 displayed on the display portion 7202, application can be started.

With the operation button 7205, a variety of functions such as time setting, power on/off, on/off of wireless communication, setting and cancellation of a silent mode, and setting and cancellation of a power saving mode can be performed. For example, the functions of the operation button 7205 can be set freely by setting the operating system incorporated in the portable information terminal 7200.

The portable information terminal 7200 can perform near field communication that is standardized communication. For example, mutual communication between the portable information terminal 7200 and a headset capable of wireless communication enables hands-free calling.

The portable information terminal 7200 includes the input/output terminal 7206, and data can be directly transmitted to and received from another information terminal via a connector. In addition, charging via the input/output terminal 7206 is possible. Note that the charge operation may be performed by wireless power feeding without using the input/output terminal 7206.

The display portion 7202 of the portable information terminal 7200 includes the secondary battery of one embodiment of the present invention. When the secondary battery of one embodiment of the present invention is used, a lightweight portable information terminal with a long lifetime can be provided. To improve safety, a protection circuit that prevents overcharging and/or overdischarging of the secondary battery may be electrically connected to the secondary battery. For example, the secondary battery 7104 illustrated in FIG. 29E that is in the state of being curved can be provided in the housing 7201. Alternatively, the secondary battery 7104 illustrated in FIG. 29E can be provided in the band 7203 such that it can be curved.

The portable information terminal 7200 preferably includes a sensor. As the sensor, for example, a human body sensor such as a fingerprint sensor, a pulse sensor, or a temperature sensor, a touch sensor, a pressure sensitive sensor, or an acceleration sensor is preferably mounted.

FIG. 29G shows an example of an armband display device. A display device 7300 includes a display portion 7304 and the secondary battery of one embodiment of the present invention. To improve safety, a protection circuit that prevents overcharging and/or overdischarging of the secondary battery may be electrically connected to the secondary battery. The display device 7300 can include a touch sensor in the display portion 7304 and can serve as a portable information terminal.

The display surface of the display portion 7304 is curved, and images can be displayed on the curved display surface. A display state of the display device 7300 can be changed by, for example, near field communication that is standardized communication.

The display device 7300 includes an input/output terminal, and data can be directly transmitted to and received from another information terminal via a connector. In addition, charging via the input/output terminal is possible. Note that the charge operation may be performed by wireless power feeding without using the input/output terminal.

When the secondary battery of one embodiment of the present invention is used as the secondary battery included in the display device 7300, a lightweight display device with a long lifetime can be provided.

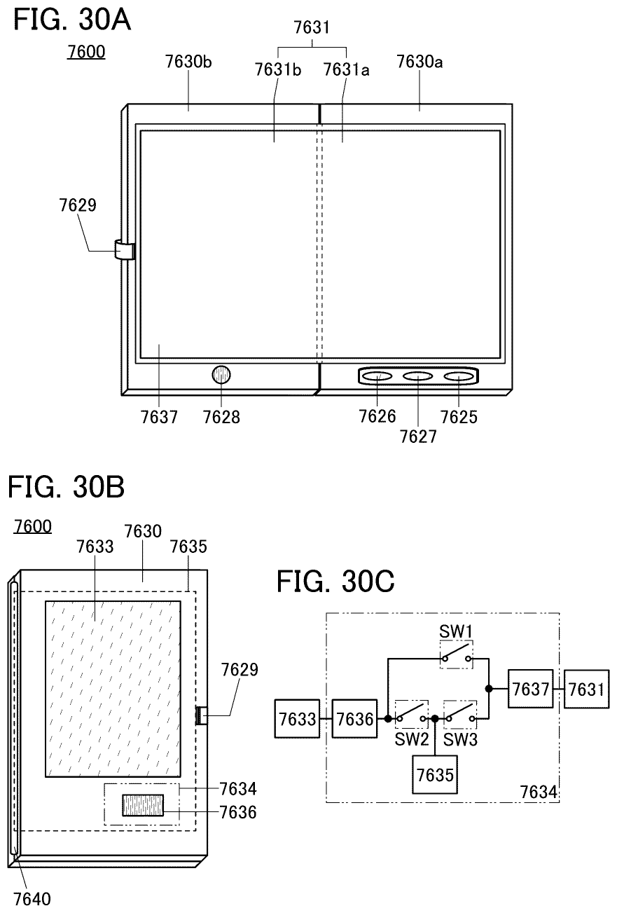

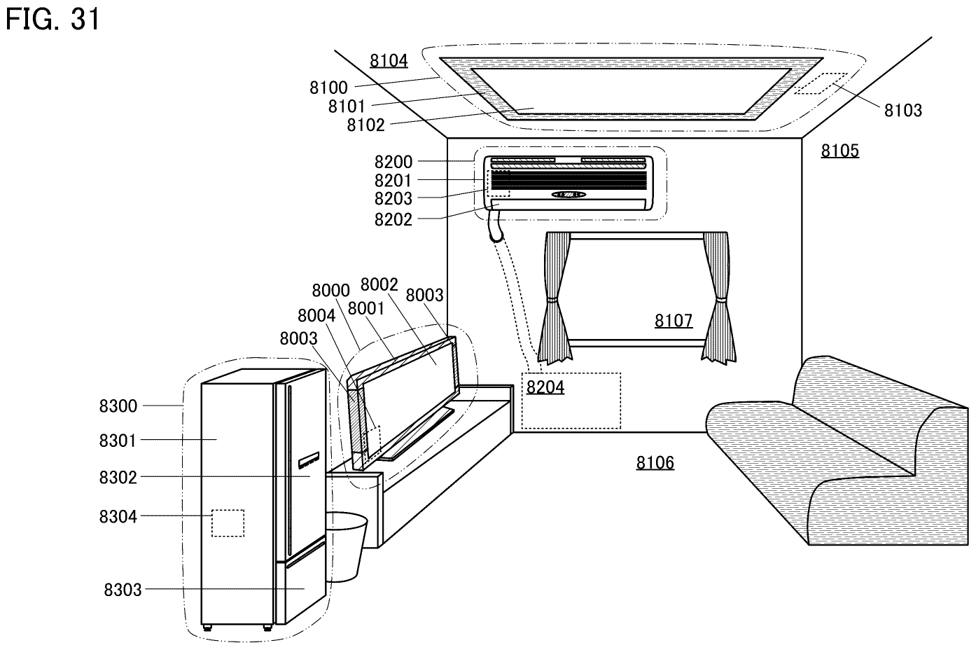

Examples of electronic devices each including the secondary battery of one embodiment of the present invention with excellent cycle performance are described with reference to FIG. 29H, FIG. 30, and FIG. 31.

When the secondary battery of one embodiment of the present invention is used as a secondary battery of an electronic device, a lightweight product with a long lifetime can be provided. Examples of the daily electronic device include an electric toothbrush, an electric shaver, and electric beauty equipment. As secondary batteries of these products, small and lightweight stick type secondary batteries with high capacity are desired in consideration of handling ease for users.

FIG. 29H is a perspective view of a device called a cigarette smoking device (electronic cigarette). In FIG. 29H, an electronic cigarette 7500 includes an atomizer 7501 including a heating element, a secondary battery 7504 that supplies electric power to the atomizer, and a cartridge 7502 including a liquid supply bottle, a sensor, or the like. To improve safety, a protection circuit that prevents overcharging and/or overdischarging of the secondary battery 7504 may be electrically connected to the secondary battery 7504. The secondary battery 7504 illustrated in FIG. 29H includes an external terminal for connection to a charger. When the electronic cigarette 7500 is held, the secondary battery 7504 is a tip portion; thus, it is preferred that the secondary battery 7504 have a short total length and be lightweight. With the secondary battery of one embodiment of the present invention, which has high capacity and excellent cycle performance, the small and lightweight electronic cigarette 7500 that can be used for a long time over a long period can be provided.

Next, FIG. 30A and FIG. 30B show an example of a tablet terminal that can be folded in half. A tablet terminal 7600 illustrated in FIG. 30A and FIG. 30B includes a housing 7630a, a housing 7630b, a movable portion 7640 connecting the housing 7630a and the housing 7630b to each other, a display portion 7631 including a display portion 7631a and a display portion 7631b, a switch 7625 to a switch 7627, a fastener 7629, and an operation switch 7628. A flexible panel is used for the display portion 7631, whereby a tablet terminal with a larger display portion can be provided. FIG. 30A illustrates the tablet terminal 7600 that is opened, and FIG. 30B illustrates the tablet terminal 7600 that is closed.

The tablet terminal 7600 includes a power storage unit 7635 inside the housing 7630a and the housing 7630b. The power storage unit 7635 is provided across the housing 7630a and the housing 7630b, passing through the movable portion 7640.

The entire region or part of the region of the display portion 7631 can be a touch panel region, and data can be input by touching text, an input form, an image including an icon, and the like displayed on the region. For example, it is possible that keyboard buttons are displayed on the entire display portion 7631a on the housing 7630a side, and data such as text or an image is displayed on the display portion 7631b on the housing 7630b side.

It is possible that a keyboard is displayed on the display portion 7631b on the housing 7630b side, and data such as text or an image is displayed on the display portion 7631a on the housing 7630a side. Furthermore, it is possible that a switching button for showing/hiding a keyboard on a touch panel is displayed on the display portion 7631 and the button is touched with a finger, a stylus, or the like to display a keyboard on the display portion 7631.

Touch input can be performed concurrently in a touch panel region in the display portion 7631a on the housing 7630a side and a touch panel region in the display portion 7631b on the housing 7630b side.

The switch 7625 to the switch 7627 may function not only as an interface for operating the tablet terminal 7600 but also as an interface that can switch various functions. For example, at least one of the switch 7625 to the switch 7627 may function as a switch for switching power on/off of the tablet terminal 7600. For another example, at least one of the switch 7625 to the switch 7627 may have a function of switching the display orientation between a portrait mode and a landscape mode or a function of switching display between monochrome display and color display. For another example, at least one of the switch 7625 to the switch 7627 may have a function of adjusting the luminance of the display portion 7631. The luminance of the display portion 7631 can be optimized in accordance with the amount of external light in use of the tablet terminal 7600 detected by an optical sensor incorporated in the tablet terminal 7600. Note that another sensing device including a sensor for sensing inclination, such as a gyroscope sensor or an acceleration sensor, may be incorporated in the tablet terminal 7600, in addition to the optical sensor.

FIG. 30A shows an example in which the display portion 7631a on the housing 7630a side and the display portion 7631b on the housing 7630b side have substantially the same display area; however, there is no particular limitation on the display areas of the display portion 7631a and the display portion 7631b, and the display portions may have different sizes or different display quality. For example, one may be a display panel that can display higher-definition images than the other.

The tablet terminal 7600 is folded in half in FIG. 30B. The tablet terminal 7600 includes a housing 7630, a solar cell 7633, and a charging and discharging control circuit 7634 including a DCDC converter 7636. The secondary battery of one embodiment of the present invention is used as the power storage unit 7635.

Note that as described above, the tablet terminal 7600 can be folded in half, and thus can be folded when not in use such that the housing 7630a and the housing 7630b overlap with each other. By the folding, the display portion 7631 can be protected, which increases the durability of the tablet terminal 7600. With the power storage unit 7635 including the secondary battery of one embodiment of the present invention, which has high capacity and excellent cycle performance, the tablet terminal 7600 that can be used for a long time over a long period can be provided. To improve safety, a protection circuit that prevents overcharging and/or overdischarging of the secondary battery included in the power storage unit 7635 may be electrically connected to the secondary battery.

In addition, the tablet terminal 7600 illustrated in FIG. 30A and FIG. 30B can also have a function of displaying various kinds of data (e.g., a still image, a moving image, and a text image), a function of displaying a calendar; a date, or the time on the display portion, a touch-input function of operating or editing data displayed on the display portion by touch input, a function of controlling processing by various kinds of software (programs), and the like.

The solar cell 7633, which is attached on the surface of the tablet terminal 7600, can supply electric power to a touch panel, a display portion, a video signal processing portion, and the like. Note that the solar cell 7633 can be provided on one surface or both surfaces of the housing 7630 and the power storage unit 7635 can be charged efficiently. The use of a lithium-ion battery as the power storage unit 7635 brings an advantage such as a reduction in size.

The structure and operation of the charging and discharging control circuit 7634 illustrated in FIG. 30B are described with reference to a block diagram in FIG. 30C. The solar cell 7633, the power storage unit 7635, the DCDC converter 7636, a converter 7637, a switch SW1 to a switch SW3, and the display portion 7631 are illustrated in FIG. 30C, and the power storage unit 7635, the DCDC converter 7636, the converter 7637, and the switch SW1 to the switch SW3 correspond to the charging and discharging control circuit 7634 illustrated in FIG. 30B.

First, an operation example in which electric power is generated by the solar cell 7633 using external light is described. The voltage of electric power generated by the solar cell is raised or lowered by the DCDC converter 7636 to a voltage for charging the power storage unit 7635. When the display portion 7631 is operated with the electric power from the solar cell 7633, the switch SW1 is turned on and the voltage is raised or lowered by the converter 7637 to a voltage needed for the display portion 7631. When display on the display portion 7631 is not performed, the switch SW1 is turned off and the switch SW2 is turned on, so that the power storage unit 7635 is charged.

Note that the solar cell 7633 is described as an example of a power generation unit; however, one embodiment of the present invention is not limited to this example. The power storage unit 7635 may be charged using another power generation unit such as a piezoelectric element or a thermoelectric conversion element (Peltier element). For example, the charging may be performed with a non-contact electric power transmission module that performs charging by transmitting and receiving electric power wirelessly (without contact), or with a combination of other charge units.

FIG. 31 illustrates other examples of electronic devices. In FIG. 31, a display device 8000 is an example of an electronic device including a secondary battery 8004 of one embodiment of the present invention. Specifically, the display device 8000 corresponds to a display device for TV broadcast reception and includes a housing 8001, a display portion 8002, speaker portions 8003, the secondary battery 8004, and the like. To improve safety, a protection circuit that prevents overcharging and/or overdischarging of the secondary battery 8004 may be electrically connected to the secondary battery 8004. The secondary battery 8004 of one embodiment of the present invention is provided in the housing 8001. The display device 8000 can be supplied with electric power from a commercial power source and can use electric power stored in the secondary battery 8004. Thus, the display device 8000 can be operated with the use of the secondary battery 8004 of one embodiment of the present invention as an uninterruptible power source even when electric power cannot be supplied from a commercial power source due to power failure or the like.

A semiconductor display device such as a liquid crystal display device, a light-emitting device in which a light-emitting element such as an organic EL element is provided in each pixel, an electrophoresis display device, a DMD (Digital Micromirror Device), a PDP (Plasma Display Panel), or an FED (Field Emission Display) can be used for the display portion 8002.

Note that the display device includes, in its category, all of information display devices for personal computers, advertisement displays, and the like besides information display devices for TV broadcast reception.

In FIG. 31, an installation lighting device 8100 is an example of an electronic device including a secondary battery 8103 of one embodiment of the present invention. Specifically, the lighting device 8100 includes a housing 8101, a light source 8102, the secondary battery 8103, and the like. To improve safety, a protection circuit that prevents overcharging and/or overdischarging of the secondary battery 8103 may be electrically connected to the secondary battery 8103. Although FIG. 31 illustrates the case where the secondary battery 8103 is provided in a ceiling 8104 on which the housing 8101 and the light source 8102 are installed, the secondary battery 8103 may be provided in the housing 8101. The lighting device 8100 can be supplied with electric power from a commercial power source and can use electric power stored in the secondary battery 8103. Thus, the lighting device 8100 can be operated with the use of the secondary battery 8103 of one embodiment of the present invention as an uninterruptible power source even when electric power cannot be supplied from a commercial power source due to power failure or the like.

Note that although the installation lighting device 8100 provided in the ceiling 8104 is illustrated in FIG. 31 as an example, the secondary battery of one embodiment of the present invention can be used in an installation lighting device provided in, for example, a side wall 8105, a floor 8106, or a window 8107 other than the ceiling 8104, and can be used in a tabletop lighting device or the like.

As the light source 8102, an artificial light source that emits light artificially by using electric power can be used. Specifically, an incandescent lamp, a discharge lamp such as a fluorescent lamp, and light-emitting elements such as an LED and/or an organic EL element are given as examples of the artificial light source.

In FIG. 31, an air conditioner including an indoor unit 8200 and an outdoor unit 8204 is an example of an electronic device including a secondary battery 8203 of one embodiment of the present invention. Specifically, the indoor unit 8200 includes a housing 8201, an air outlet 8202, the secondary battery 8203, and the like. To improve safety, a protection circuit that prevents overcharging and/or overdischarging of the secondary battery 8203 may be electrically connected to the secondary battery 8203. Although FIG. 31 illustrates the case where the secondary battery 8203 is provided in the indoor unit 8200, the secondary battery 8203 may be provided in the outdoor unit 8204. Alternatively, the secondary batteries 8203 may be provided in both the indoor unit 8200 and the outdoor unit 8204. The air conditioner can be supplied with electric power from a commercial power source and can use electric power stored in the secondary battery 8203. Particularly in the case where the secondary batteries 8203 are provided in both the indoor unit 8200 and the outdoor unit 8204, the air conditioner can be operated with the use of the secondary battery 8203 of one embodiment of the present invention as an uninterruptible power source even when electric power cannot be supplied from a commercial power source due to power failure or the like.

Note that although the split-type air conditioner including the indoor unit and the outdoor unit is illustrated in FIG. 31 as an example, the secondary battery of one embodiment of the present invention can be used in an air conditioner in which the function of an indoor unit and the function of an outdoor unit are integrated in one housing.

In FIG. 31, an electric refrigerator-freezer 8300 is an example of an electronic device including a secondary battery 8304 of one embodiment of the present invention. Specifically, the electric refrigerator-freezer 8300 includes a housing 8301, a refrigerator door 8302, a freezer door 8303, the secondary battery 8304, and the like. To improve safety, a protection circuit that prevents overcharging and/or overdischarging of the secondary battery 8304 may be electrically connected to the secondary battery 8304. The secondary battery 8304 is provided in the housing 8301 in FIG. 31. The electric refrigerator-freezer 8300 can be supplied with electric power from a commercial power source and can use electric power stored in the secondary battery 8304. Thus, the electric refrigerator-freezer 8300 can be operated with the use of the secondary battery 8304 of one embodiment of the present invention as an uninterruptible power source even when electric power cannot be supplied from a commercial power source due to power failure or the like.

Note that among the electronic devices described above, a high-frequency heating apparatus such as a microwave oven and an electronic device such as an electric rice cooker require high electric power in a short time. Therefore, the tripping of a breaker of a commercial power source in use of the electronic device can be prevented by using the secondary battery of one embodiment of the present invention as an auxiliary power source for supplying electric power which cannot be supplied enough by a commercial power source.

In a time period when electronic devices are not used, particularly when the proportion of the amount of electric power which is actually used to the total amount of electric power which can be supplied from a commercial power supply source (such a proportion is referred to as a usage rate of electric power) is low, electric power is stored in the secondary battery, whereby an increase in the usage rate of electric power can be inhibited in a time period other than the above time period. For example, in the case of the electric refrigerator-freezer 8300, electric power is stored in the secondary battery 8304 in night time when the temperature is low and the refrigerator door 8302 and the freezer door 8303 are not opened or closed. Moreover, in daytime when the temperature is high and the refrigerator door 8302 and the freezer door 8303 are opened and closed, the usage rate of electric power in daytime can be kept low by using the secondary battery 8304 as an auxiliary power source.

According to one embodiment of the present invention, the secondary battery can have excellent cycle performance and improved reliability. Furthermore, according to one embodiment of the present invention, a secondary battery with high capacity can be obtained; thus, the secondary battery itself can be made more compact and lightweight as a result of improved characteristics of the secondary battery. Thus, the secondary battery of one embodiment of the present invention is used in the electronic device described in this embodiment, whereby a more lightweight electronic device with a longer lifetime can be obtained.

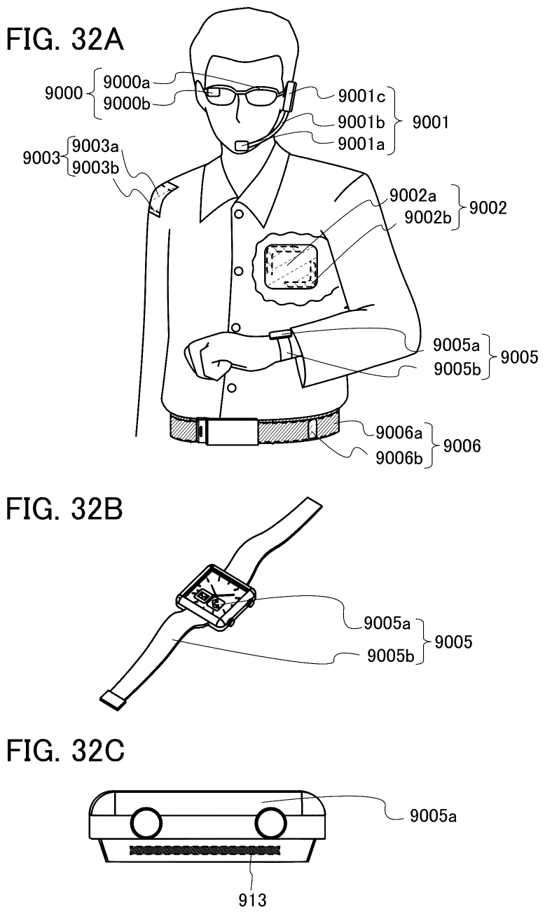

FIG. 32A shows examples of wearable devices. A secondary battery is used as a power source of a wearable device. To have improved splash resistance, water resistance, or dust resistance in daily use or outdoor use by a user, a wearable device is desirably capable of being charged with and without a wire whose connector portion for connection is exposed.

For example, the secondary battery of one embodiment of the present invention can be provided in a glasses-type device 9000 illustrated in FIG. 32A. The glasses-type device 9000 includes a frame 9000a and a display part 9000b. The secondary battery is provided in a temple of the frame 9000a having a curved shape, whereby the glasses-type device 9000 can be lightweight, can have a well-balanced weight, and can be used continuously for a long time. To improve safety, a protection circuit that prevents overcharging and/or overdischarging of the secondary battery may be electrically connected to the secondary battery. With the use of the secondary battery of one embodiment of the present invention, space saving required with downsizing of a housing can be achieved.

The secondary battery of one embodiment of the present invention can be provided in a headset-type device 9001. The headset-type device 9001 includes at least a microphone part 9001a, a flexible pipe 9001b, and an earphone portion 9001c. The secondary battery can be provided in the flexible pipe 9001b or the earphone portion 9001c. To improve safety, a protection circuit that prevents overcharging and/or overdischarging of the secondary battery may be electrically connected to the secondary battery. With the use of the secondary battery of one embodiment of the present invention, space saving required with downsizing of a housing can be achieved.

The secondary battery of one embodiment of the present invention can be provided in a device 9002 that can be attached directly to a body. A secondary battery 9002b can be provided in a thin housing 9002a of the device 9002. To improve safety, a protection circuit that prevents overcharging and/or overdischarging of the secondary battery 9002b may be electrically connected to the secondary battery 9002b. With the use of the secondary battery of one embodiment of the present invention, space saving required with downsizing of a housing can be achieved.

The secondary battery of one embodiment of the present invention can be provided in a device 9003 that can be attached to clothes. A secondary battery 9003b can be provided in a thin housing 9003a of the device 9003. To improve safety, a protection circuit that prevents overcharging and/or overdischarging of the secondary battery 9003b may be electrically connected to the secondary battery 9003b. With the use of the secondary battery of one embodiment of the present invention, space saving required with downsizing of a housing can be achieved.

The secondary battery of one embodiment of the present invention can be provided in a belt-type device 9006. The belt-type device 9006 includes a belt portion 9006a and a wireless power feeding and receiving portion 9006b, and the secondary battery can be provided inside the belt portion 9006a. To improve safety, a protection circuit that prevents overcharging and/or overdischarging of the secondary battery may be electrically connected to the secondary battery. With the use of the secondary battery of one embodiment of the present invention, space saving required with downsizing of a housing can be achieved.

The secondary battery of one embodiment of the present invention can be provided in a watch-type device 9005. The watch-type device 9005 includes a display portion 9005a and a belt portion 9005b, and the secondary battery can be provided in the display portion 9005a or the belt portion 9005b. To improve safety, a protection circuit that prevents overcharging and/or overdischarging of the secondary battery may be electrically connected to the secondary battery. With the use of the secondary battery of one embodiment of the present invention, space saving required with downsizing of a housing can be achieved.

The display portion 9005a can display various kinds of information such as time and reception information of an e-mail and/or an incoming call.

In addition, the watch-type device 9005 is a wearable device that is wound around an arm directly; thus, a sensor that measures the pulse, the blood pressure, or the like of the user may be incorporated therein. Data on the exercise quantity and health of the user can be stored to be used for health maintenance.

FIG. 32B is a perspective view of the watch-type device 9005 that is detached from an arm.

FIG. 32C is a side view. FIG. 32C illustrates a state where the secondary battery 913 of one embodiment of the present invention is incorporated in the watch-type device 9005. The secondary battery 913, which is small and lightweight, overlaps with the display portion 9005a.

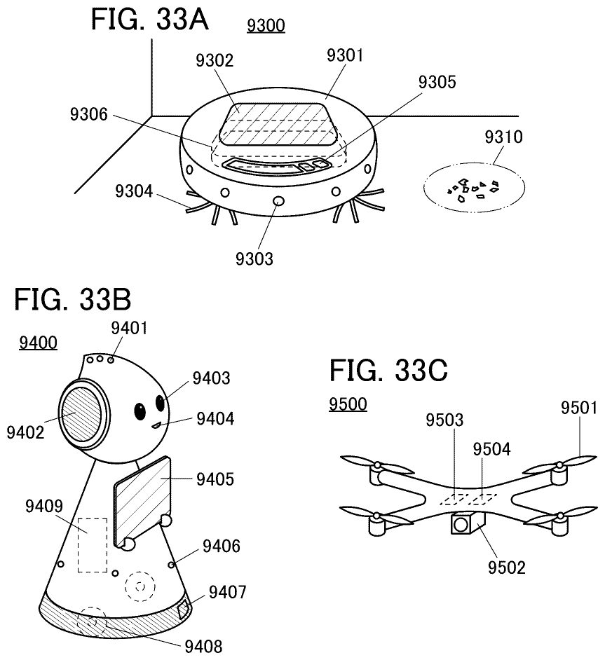

FIG. 33A shows an example of a cleaning robot. A cleaning robot 9300 includes a display portion 9302 placed on the top surface of a housing 9301, a plurality of cameras 9303 placed on the side surface of the housing 9301, a brush 9304, operation buttons 9305, a secondary battery 9306, a variety of sensors, and the like. To improve safety, a protection circuit that prevents overcharging and/or overdischarging of the secondary battery 9306 may be electrically connected to the secondary battery 9306. Although not illustrated, the cleaning robot 9300 is provided with a tire, an inlet, and the like. The cleaning robot 9300 is self-propelled, detects dust 9310, and sucks up the dust through the inlet provided on the bottom surface.

For example, the cleaning robot 9300 can determine whether there is an obstacle such as a wall, furniture, or a step by analyzing images taken by the cameras 9303. In the case where the cleaning robot 9300 detects an object, such as a wire, that is likely to be caught in the brush 9304 by image analysis, the rotation of the brush 9304 can be stopped. The cleaning robot 9300 includes a secondary battery 9306 of one embodiment of the present invention and a semiconductor device or an electronic component. The cleaning robot 9300 including the secondary battery 9306 of one embodiment of the present invention can be a highly reliable electronic device that can operate for a long time.

FIG. 33B shows an example of a robot. A robot 9400 illustrated in FIG. 33B includes a secondary battery 9409, an illuminance sensor 9401, a microphone 9402, an upper camera 9403, a speaker 9404, a display portion 9405, a lower camera 9406, an obstacle sensor 9407, a moving mechanism 9408, an arithmetic device, and the like. To improve safety, a protection circuit that prevents overcharging and/or overdischarging of the secondary battery 9409 may be electrically connected to the secondary battery 9409.

The microphone 9402 has a function of detecting a speaking voice of a user, an environmental sound, and the like. The speaker 9404 has a function of outputting sound. The robot 9400 can communicate with a user using the microphone 9402 and the speaker 9404.

The display portion 9405 has a function of displaying various kinds of information. The robot 9400 can display information desired by a user on the display portion 9405. The display portion 9405 may be provided with a touch panel. Moreover, the display portion 9405 may be a detachable information terminal, in which case charging and data communication can be performed when the display portion 9405 is set at the home position of the robot 9400.

The upper camera 9403 and the lower camera 9406 each have a function of taking an image of the surroundings of the robot 9400. The obstacle sensor 9407 can detect, with the use of the moving mechanism 9408, the presence of an obstacle in the direction where the robot 9400 advances. The robot 9400 can move safely by recognizing the surroundings with the upper camera 9403, the lower camera 9406, and the obstacle sensor 9407.

The robot 9400 includes the secondary battery 9409 of one embodiment of the present invention and a semiconductor device or an electronic component. The robot 9400 including the secondary battery of one embodiment of the present invention can be a highly reliable electronic device that can operate for a long time.

FIG. 33C shows an example of a flying object. A flying object 9500 illustrated in FIG. 33C includes propellers 9501, a camera 9502, a secondary battery 9503, and the like and has a function of flying autonomously. To improve safety, a protection circuit that prevents overcharging and/or overdischarging of the secondary battery 9503 may be electrically connected to the secondary battery 9503.

For example, image data taken by the camera 9502 is stored in an electronic component 9504. The electronic component 9504 can analyze the image data to detect whether there is an obstacle in the way of the movement. Moreover, the electronic component 9504 can estimate the remaining battery level from a change in the power storage capacity of the secondary battery 9503. The flying object 9500 includes the secondary battery 9503 of one embodiment of the present invention. The flying object 9500 including the secondary battery of one embodiment of the present invention can be a highly reliable electronic device that can operate for a long time.

This embodiment can be implemented in appropriate combination with the other embodiments.

(Notes on Description of this Specification and the Like)

The description of the above embodiments and each structure in the embodiments are noted below.

One embodiment of the present invention can be constituted by combining, as appropriate, the structure described in each embodiment with the structures described in the other embodiments. In addition, in the case where a plurality of structure examples are described in one embodiment, the structure examples can be combined as appropriate.

Note that content (or part of the content) described in one embodiment can be applied to, combined with, or replaced with another content (or part of the content) described in the embodiment and/or content (or part of the content) described in another embodiment or other embodiments.

Note that in each embodiment, a content described in the embodiment is a content described with reference to a variety of drawings or a content described with text disclosed in the specification.

Note that by combining a diagram (or part thereof) described in one embodiment with another part of the diagram, a different diagram (or part thereof) described in the embodiment, and/or a diagram (or part thereof) described in another embodiment or other embodiments, much more diagrams can be formed.

In addition, in this specification and the like, components are classified on the basis of the functions, and shown as blocks independent of one another in block diagrams. However, in an actual circuit or the like, it is difficult to separate components on the basis of the functions, and there is such a case where one circuit is associated with a plurality of functions or a case where a plurality of circuits are associated with one function. Therefore, blocks in the block diagrams are not limited by the components described in this specification, and the description can be changed appropriately depending on the situation.

In drawings, the size, the layer thickness, or the region is shown arbitrarily for description convenience. Therefore, they are not limited to the illustrated scale. Note that the drawings are schematically shown for clarity, and embodiments of the present invention are not limited to shapes, values, or the like shown in the drawings. For example, variation in signal, voltage, or current due to noise or variation in signal, voltage, or current due to difference in timing can be included.

In this specification and the like, expressions “one of a source and a drain” (or a first electrode or a first terminal) and “the other of the source and the drain” (or a second electrode or a second terminal) are used in the description of the connection relationship of a transistor. This is because a source and a drain of a transistor are interchangeable depending on the structure, operation conditions, or the like of the transistor. Note that the source or the drain of the transistor can also be referred to as a source (or drain) terminal, a source (or drain) electrode, or the like as appropriate depending on the situation.

In addition, in this specification and the like, the terms “electrode” and “wiring” do not functionally limit these components. For example, an “electrode” is used as part of a wiring in some cases, and vice versa. Furthermore, the term “electrode” or “wiring” also includes the case where a plurality of “electrodes”, a plurality of “wirings”, or a plurality of “electrodes” and a plurality of “wirings” are formed in an integrated manner, for example.

In this specification and the like, voltage and potential can be replaced with each other as appropriate. The voltage refers to a potential difference from a reference potential, and when the reference potential is a ground voltage, for example, the voltage can be rephrased into the potential. The ground potential does not necessarily mean 0 V. Note that potentials are relative, and the potential supplied to a wiring or the like is changed depending on the reference potential, in some cases.

Note that in this specification and the like, the terms “film”, “layer”, and the like can be interchanged with each other depending on the case or according to circumstances. For example, the term “conductive layer” can be changed into the term “conductive film” in some cases. As another example, the term “insulating film” can be changed into the term “insulating layer” in some cases.

In this specification and the like, a switch has a function of controlling whether current flows or not by being in a conduction state (an on state) or a non-conduction state (an off state). Alternatively, a switch has a function of selecting and changing a current path.

In this specification and the like, channel length refers to, for example, the distance between a source and a drain in a region where a semiconductor (or a portion where current flows in a semiconductor when a transistor is in an on state) and a gate overlap with each other or a region where a channel is formed in a top view of the transistor.

In this specification and the like, channel width refers to, for example, the length of a portion where a source and a drain face each other in a region where a semiconductor (or a portion where current flows in a semiconductor when a transistor is in an on state) and a gate electrode overlap with each other or a region where a channel is formed.

In this specification and the like, the expression “A and B are connected” includes the case where A and B are electrically connected as well as the case where A and B are directly connected. Here, the expression “A and B are electrically connected” means the case where electric signals can be transmitted and received between A and B when an object having any electric action exists between A and B.

### Example 1

In this example, secondary batteries of embodiments of the present invention were fabricated and evaluated.

### [Formation of Positive Electrode Active Material]

Positive electrode active materials were formed with reference to the formation method shown in FIG. 6.

As LiMO₂ in Step S14, with the use of cobalt as the transition metal M, commercially available lithium cobalt oxide (Cellseed C-10N manufactured by NIPPON CHEMICAL INDUSTRIAL CO., LTD.) not containing any additive was prepared. Here, lithium fluoride and magnesium fluoride were prepared as the X1 source as in Step S20a and the lithium fluoride and the magnesium fluoride were mixed by a solid phase method as in Step S31. Lithium fluoride and magnesium fluoride were added such that the number of molecules of lithium fluoride was 0.33 and the number of molecules of magnesium fluoride was 1 with the number of cobalt atoms assumed as 100. As in Step S32, the mixture here is the mixture 903.

Next, annealing was performed in a manner similar to that of Step S33. In a square-shaped alumina container, 30 g of the mixture 903 was placed, a lid was put on the container, and heating was performed in a muffle furnace. The atmosphere in the furnace was purged and an oxygen gas was introduced; the oxygen flow was stopped during the heating. The annealing temperature was 900° C., and the annealing time was 20 hours.

To the composite oxide that had been heated, nickel hydroxide and aluminum hydroxide were added and mixed by a dry method in Step S51. The addition was performed so that the number of nickel atoms was 0.5 and the number of aluminum atoms was 0.5 with the number of cobalt atoms assumed as 100. The mixture here is the mixture 904.

Next, annealing was performed in a manner similar to that of Step S33. In a square-shaped alumina container, 30 g of the mixture 903 was placed, a lid was put on the container, and heating was performed in a muffle furnace. The atmosphere in the furnace was purged and an oxygen gas was introduced; the oxygen flow was performed during the heating. The annealing temperature was 850° C., and the annealing time was 10 hours.

After that, the mixture was made to pass through a sieve with 53 μmϕ and powder was collected, so that positive electrode active materials were obtained.

### [Formation of Positive Electrode]

Next, positive electrodes were formed using the positive electrode active material formed in the above manner. The positive electrode active material formed in the above manner, acetylene black (AB), and polyvinylidene fluoride (PVDF) were mixed at the positive electrode active material:AB:PVDF=95:3:2 (weight ratio) using NMP as a solvent, whereby slurry was formed. After a current collector was coated with the formed slurry, the solvent was volatilized. After that, at 120° C., a pressure of 120 kN/m was applied and a positive electrode active material layer was formed on the current collector; thus, each positive electrode was formed. Aluminum foil having a thickness of 20 μm was used as the current collector. The positive electrode active material layer was provided on one surface of the current collector. The carried amount was approximately 10 mg/cm².

### [Formation of Negative Electrode]

Negative electrodes were formed using graphite as a negative electrode active material.

MCMB graphite having a specific surface area of 1.5 m²/g was used as the graphite and mixed with a conductive agent, CMC-Na, and SBR at the graphite: the conductive agent:CMC-Na:SBR=96:1:1:2 (weight ratio) using water as a solvent, whereby slurry was formed.

The polymerization degree of CMC-Na that was used was 600 to 800, and the viscosity of a 1 weight % CMC-Na aqueous solution was in the range from 300 mPa·s to 500 mPa·s. As the conductive agent, VGCF (registered trademark)-H (manufactured by SHOWA DENKO K.K., the fiber diameter: 150 nm, the specific surface area: 13 m²/g) was used.

Current collectors were coated with the corresponding formed slurry and then drying was performed, and negative electrode active material layers were formed on the current collectors. As the current collector, copper foil having a thickness of 18 μm was used. The negative electrode active material layer was provided on both surfaces or one surface of the current collector. The carried amount was approximately 9 mg/cm².

### [Fabrication of Secondary Batteries]

With use of the positive electrodes and the negative electrodes formed in the above manner, the secondary batteries using films as exterior bodies were fabricated.

As a separator, 23-μm-thick polyimide was used.

For a secondary battery including an electrolyte solution A described later, one negative electrode in which negative electrode active material layers are formed on both surfaces and two positive electrodes in each of which a positive electrode active material layer is formed on one surface were prepared. The positive electrode active material layers were arranged so as to face the respective negative electrode active material layers formed on the both surfaces of the negative electrode with the separator sandwiched therebetween.

For a secondary battery including an electrolyte solution B described later, one negative electrode in which a negative electrode active material layer is formed on one surface and one positive electrode in which a positive electrode active material layer is formed on one surface were prepared. The negative electrode active material layer and the positive electrode active material layer were arranged so as to face each other with the separator sandwiched therebetween.

Leads were bonded to the positive electrode and the negative electrode.

A stack in which the positive electrodes, the negative electrode, and the separators are stacked was sandwiched between facing portions of the exterior body that is folded in half, and the stack was placed so that one ends of the leads extend outside the exterior body. Next, one side of the exterior body was left as an aperture, and the other sides were sealed.

As a film to be the exterior body, a film in which a polypropylene layer, an acid modified polypropylene layer, an aluminum layer, and a nylon layer are stacked in this order was used. The thickness of the film was approximately 110 nμm. The film to be the exterior body was bent so that the nylon layer is placed as the surface of the exterior body placed on the outer side and the polypropylene layer is placed as the surface of the exterior body placed on the inner side. The thickness of the aluminum layer was approximately 40 μm, the thickness of the nylon layer was approximately 25 μm, and the total thickness of the polypropylene layer and the acid modified polypropylene layer was approximately 45 μm.

Next, in an argon gas atmosphere, an electrolyte solution was introduced from the one side left as an aperture. Two kinds of electrolyte solutions (hereinafter, an electrolyte solution A and an electrolyte solution B) were prepared.

The electrolyte solution A was prepared. As a solvent of the electrolyte solution, EMI-FSA represented by Structural Formula (G11) was used. As a lithium salt, LiFSA (lithium bis(fluorosulfonyl)amide) was used, and the concentration of the lithium salt in the electrolyte solution was 2.15 mol/L.

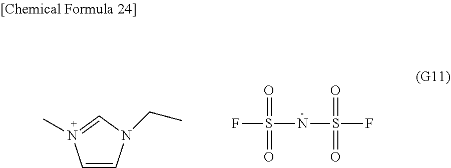

In addition, as an electrolyte solution B, which is a comparative example, an electrolyte solution including a cyclic carbonate was prepared. Specifically, as a solvent, a solution in which ethylene carbonate (EC) and diethyl carbonate (DEC) were mixed at EC:DEC=3:7 (volume ratio) was used. As a lithium salt, lithium hexafluorophosphate (LiPF₆) was used. The concentration of the lithium salt in the electrolyte solution was 1.00 mol/L.

Then, the one side of the exterior body left as an aperture was sealed in a reduced-pressure atmosphere.

Through the above steps, the secondary batteries were fabricated.

### [Aging]

Next, the secondary batteries were subjected to aging.

The secondary battery was sandwiched between two plates, CC charging was performed at 0.01 C by a capacity of 15 mAh/g, a 10-minute break was taken, and then CC charging was performed at 0.1 C by a capacity of 105 mAh/g. After that, the two plates were removed, the secondary battery was held for 24 hours at 0° C., the one side of the exterior body was cut open in an argon atmosphere, degassing was performed, and then sealing was performed again.

### [Evaluation 1 of Cycle Characteristics]

The secondary battery was sandwiched between two plates and cycle performance of the secondary battery was evaluated.

The area of the positive electrode active material layer of the positive electrode was 20.493 cm².

The loaded amount of the negative electrode active material of the negative electrode in each battery cell was adjusted so that the capacity ratio becomes approximately higher than or equal to 75% and lower than or equal to 85%. Here, the capacity ratio denotes a value representing the capacity of the positive electrode with respect to the capacity of the negative electrode by percentage. In calculation of the capacity ratio, the capacity of the negative electrode was 330 mAh/g using the weight of the negative electrode active material as a reference. Note that in the case where the negative electrode active material layers are provided on the both surfaces of the current collector, the loaded amount of the negative electrode active material was calculated by halving the total loaded amount.

Note that the areas of the positive electrode and the negative electrode were the same in the secondary battery including the electrolyte solution A.

Cycle tests were performed in environments at 0° C., 25° C., 45° C., 60° C., and −20° C.

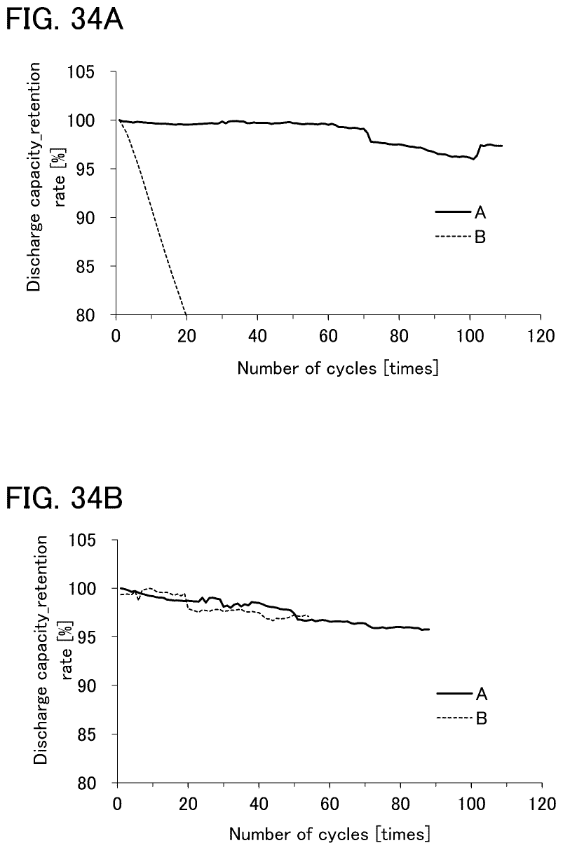

In the environment at 0° C., CCCV charging (0.2 C, a termination current of 0.1 C, 4.5 V) was performed and CC discharging (0.2 C, 3.0 V) was performed. The capacity of the secondary battery was calculated using the weight of the positive electrode active material as a reference. The C rate of the secondary battery including the electrolyte solution A was calculated on the assumption that 1 C is 210 mA/g (per weight of the positive electrode active material). The C rate of the secondary battery including the electrolyte solution B was calculated on the assumption that 1 C is 210 mA/g (per weight of the positive electrode active material). FIG. 34A shows the results of the cycle performance. The initial discharge capacity was 161.3 mAh/g for the electrolyte solution A and 145.5 mAh/g for the electrolyte solution B.

In the environment at 25° C., CCCV charging (0.2 C, a termination current of 0.1 C, 4.5 V) was performed and CC discharging (0.2 C, 3.0 V) was performed. The capacity of the secondary battery was calculated using the weight of the positive electrode active material as a reference. The C rate of the secondary battery including the electrolyte solution A was calculated on the assumption that 1 C is 210 mA/g (per weight of the positive electrode active material). The C rate of the secondary battery including the electrolyte solution B was calculated on the assumption that 1 C is 210 mA/g (per weight of the positive electrode active material). FIG. 34B shows the results of the cycle performance. The maximum value of the discharge capacity in the cycle test was 205.1 mAh/g for the electrolyte solution A and 195.0 mAh/g for the electrolyte solution B.

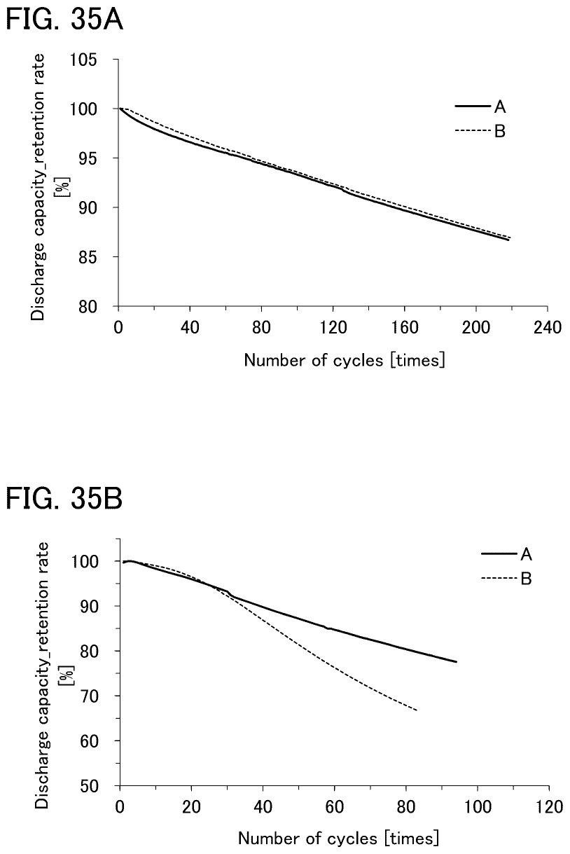

In the environment at 45° C., CCCV charging (0.5 C, a termination current of 0.2 C, 4.5 V) was performed and CC discharging (0.5 C, 3.0 V) was performed. The capacity of the secondary battery was calculated using the weight of the positive electrode active material as a reference. The C rate of the secondary battery including the electrolyte solution A was calculated on the assumption that 1 C is 210 mA/g (per weight of the positive electrode active material). The C rate of the secondary battery including the electrolyte solution B was calculated on the assumption that 1 C is 210 mA/g (per weight of the positive electrode active material). FIG. 35A shows the results of the cycle performance. The maximum value of the discharge capacity in the cycle test was 201.8 mAh/g for the electrolyte solution A and 201.0 mAh/g for the electrolyte solution B.

In the environment at 60° C., CCCV charging (0.5 C, a termination current of 0.2 C, 4.5 V) was performed and CC discharging (0.5 C, 3.0 V) was performed. The capacity of the secondary battery was calculated using the weight of the positive electrode active material as a reference. The C rate of the secondary battery including the electrolyte solution A was calculated on the assumption that 1 C is 210 mA/g (per weight of the positive electrode active material). The C rate of the secondary battery including the electrolyte solution B was calculated on the assumption that 1 C is 210 mA/g (per weight of the positive electrode active material). FIG. 35B shows the results of the cycle performance. The maximum value of the discharge capacity in the cycle test was 197.2 mAh/g for the electrolyte solution A and 213.9 mAh/g for the electrolyte solution B.

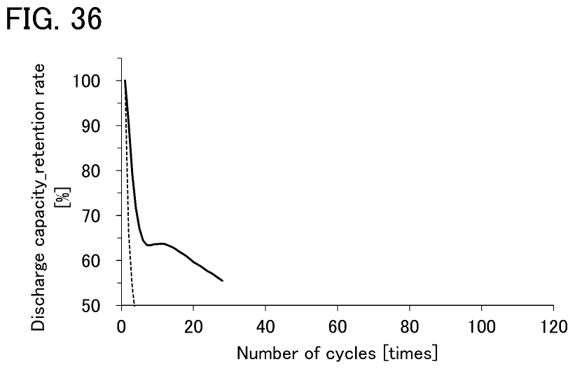

In the environment at −20° C., CCCV charging (0.1 C, a termination current of 0.05 C, 4.5 V) was performed and CC discharging (0.1 C, 3.0 V) was performed. The capacity of the secondary battery was calculated using the weight of the positive electrode active material as a reference. The C rate of the secondary battery including the electrolyte solution A was calculated on the assumption that 1 C is 210 mA/g (per weight of the positive electrode active material). The C rate of the secondary battery including the electrolyte solution B was calculated on the assumption that 1 C is 210 mA/g (per weight of the positive electrode active material). FIG. 36 shows the results of the cycle performance. The maximum value of the discharge capacity in the cycle test was 112.0 mAh/g for the electrolyte solution A and 87.2 mAh/g for the electrolyte solution B.

### Example 2

In this example, the secondary batteries that were fabricated in Example 1 and subjected to 150 or more cycles of charging and discharging at 60° C. were disassembled and the positive electrodes and the negative electrodes were observed.

The negative electrode of the secondary battery including the electrolyte solution A (2.15M LiFSA EMI-FSA) and the negative electrode of the secondary battery including the electrolyte solution B (1M LiPF₆ EC:DEC=3:7) were subjected to SEM observation. SU8030 manufactured by Hitachi High-Tech Corporation was used and an accelerating voltage was 1 kV. Cross sections were exposed by processing employing ion milling and then observed.

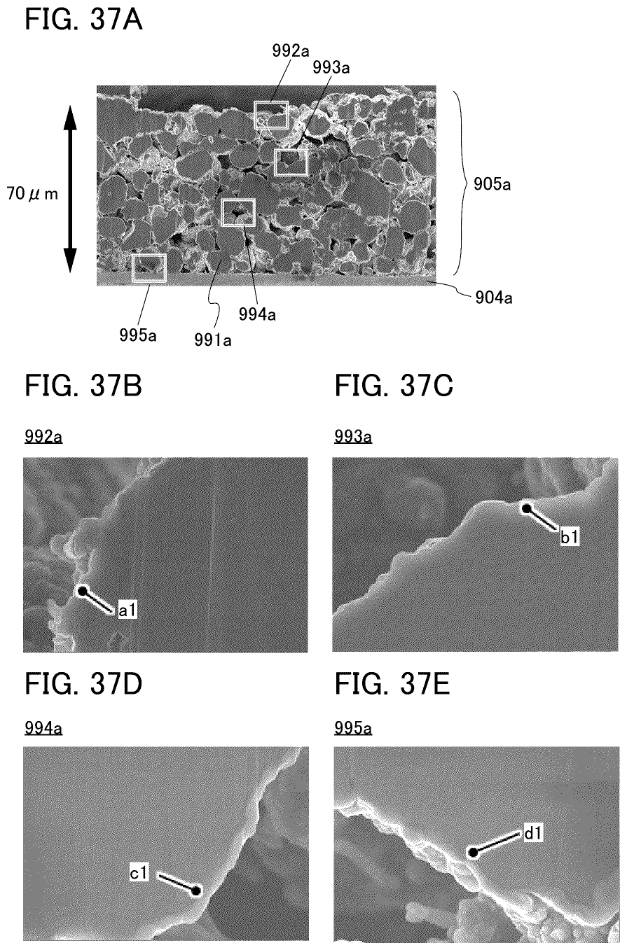

FIG. 37A is a cross-sectional SEM image of the secondary battery including the electrolyte solution A. As shown in FIG. 37A, it was observed that a negative electrode active material layer 905a including graphite 991a is provided over a current collector 904a. FIG. 37B, FIG. 37C, FIG. 37D, and FIG. 37E respectively show an enlarged view of a portion indicated by a square frame 992a shown in FIG. 37A, an enlarged view of a portion indicated by a frame 993a, an enlarged view of a portion indicated by a frame 994a, and an enlarged view of a portion indicated by a frame 995a.

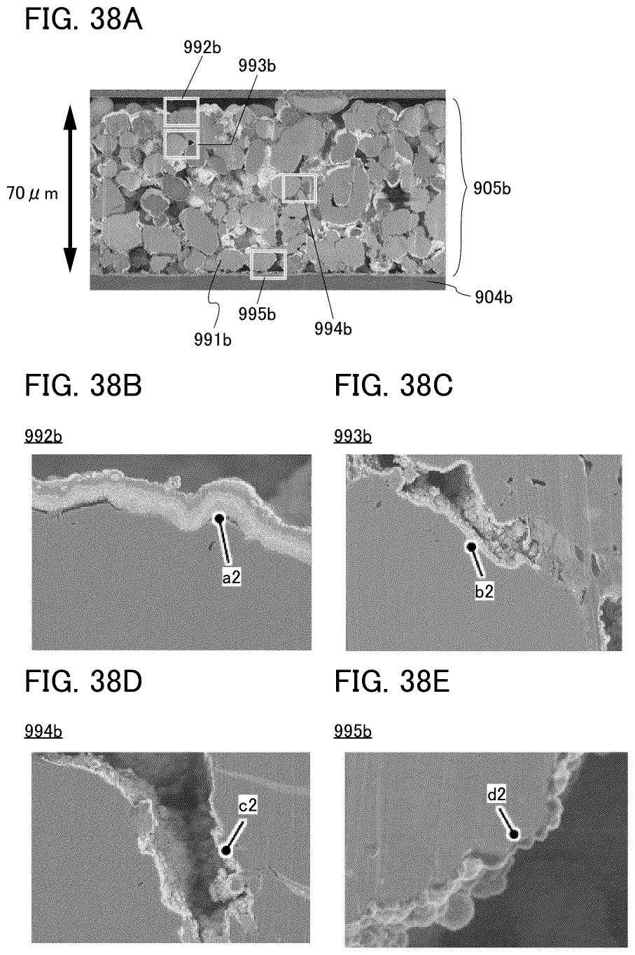

FIG. 38A is a cross-sectional SEM image of the secondary battery including the electrolyte solution B. As shown in FIG. 38A, it was observed that a negative electrode active material layer 905b including graphite 991b is provided over a current collector 904b. FIG. 38B, FIG. 38C, FIG. 38D, and FIG. 38E respectively show an enlarged view of a portion indicated by a square frame 992b shown in FIG. 38A, an enlarged view of a portion indicated by a frame 993b, an enlarged view of a portion indicated by a frame 994b, and an enlarged view of a portion indicated by a frame 995b.

In addition, EDX analysis was performed on a point a1 shown in FIG. 37B, a point b1 shown in FIG. 37C, a point c1 shown in FIG. 37D, a point d1 shown in FIG. 37E, a point a2 shown in FIG. 38B, a point b2 shown in FIG. 38C, a point c2 shown in FIG. 38D, and a point d2 shown in FIG. 38E. Moreover, EDX analysis was performed on the points shown in the diagrams. The accelerating voltage in the analysis was 5 kV.

Carbon, nitrogen, oxygen, fluorine, and sulfur were detected in the EDX analysis on the points a1, b1, c1, and d1. The amounts of magnesium, aluminum, and cobalt were less than or equal to the lower detection limit in the EDX analysis on the points a1, b1, c1, and d1. Copper was detected in the EDX analysis on the points b1, c1, and d1 and the amount of copper was less than or equal to the lower detection limit at the point a1. Copper may be derived from the current collector.

Carbon, oxygen, fluorine, and phosphorus were detected in the EDX analysis on the points a2, b2, c2, and d2. The amounts of nitrogen, magnesium, and aluminum were less than or equal to the lower detection limit in the EDX analysis on the points a2, b2, c2, and d2. Copper was detected in the EDX analysis on the point d2 and the amount of copper was less than or equal to the lower detection limit at the points a2, b2, and c2. Copper may be derived from the current collector.

Cobalt was detected in the EDX analysis on the points a2, b2, and c2. The amount of cobalt was less than or equal to the lower detection limit at the point d2. It is suggested that cobalt detected at the points a2, b2, and c2 is derived from cobalt eluted from the positive electrode active material.

As shown in FIG. 37A to FIG. 37E and FIG. 38A to FIG. 38E, a coating film was observed on a surface of the graphite. In the negative electrode of the secondary battery including the electrolyte solution A, the coating film was thin and the amount of cobalt detected by the EDX analysis was small compared with the negative electrode of the secondary battery including the electrolyte solution B. In the case of using either electrolyte solution, carbon and oxygen were detected by EDX. In the case of using the electrolyte solution A, nitrogen, fluorine, and sulfur were detected. Meanwhile, in the case of using the electrolyte solution B, fluorine and phosphorus were detected.

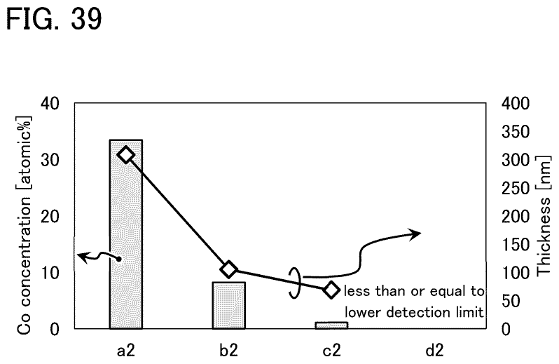

The coating film on the surface of the graphite was thicker in a portion closer to the surface of the negative electrode active material layer, i.e., farther from the current collector. The amount of cobalt detected by EDX was larger in a portion closer to the surface of the negative electrode active material layer, i.e., farther from the current collector. FIG. 39 shows the thicknesses of the coating film and the concentrations of cobalt detected by EDX in the measured regions. The thicknesses of the coating film were measured at the five positions and the average value thereof was calculated.

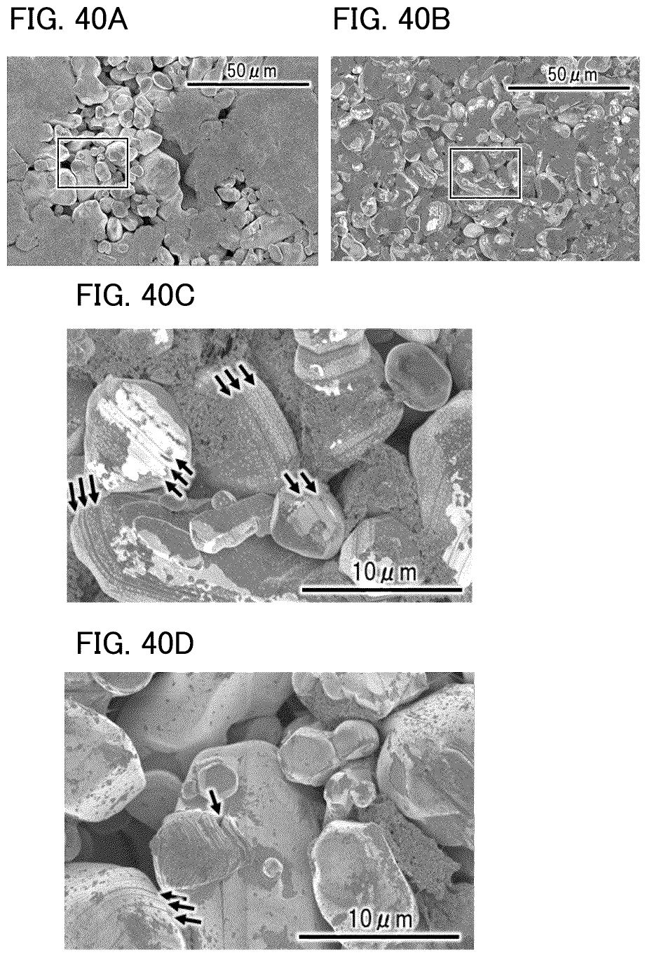

The positive electrode of the secondary battery including the electrolyte solution A and the positive electrode of the secondary battery including the electrolyte solution B were subjected to SEM observation. SU8030 manufactured by Hitachi High-Tech Corporation was used and an accelerating voltage was 1.0 kV. FIG. 40 shows SEM images. FIG. 40A and FIG. 40B respectively show a SEM observation image of the positive electrode of the secondary battery including the electrolyte solution A and the positive electrode of the secondary battery including the electrolyte solution B. FIG. 40C and FIG. 40D respectively show an enlarged view of a region indicated by a square frame in FIG. 40A and an enlarged view of a region indicated by a square frame in FIG. 40B.

At positions indicated by arrows in FIG. 40C and FIG. 40D, pits were observed. As shown in FIG. 40C and FIG. 40D, it was found that the positive electrode of the secondary battery of one embodiment of the present invention including the electrolyte solution A includes a small number of pits. This suggests that in the structure of the secondary battery including an ionic liquid for the electrolyte solution, elution of cobalt was suppressed and generation of a pit was suppressed.

### REFERENCE NUMERALS

51: positive electrode active material particle, 52: depression, 53: barrier film, 54: pit, 55: crystal plane, 56: barrier film, 57: crack, 58: pit, 100: positive electrode active material, 130: stack, 131: stack, 400: negative electrode active material, 401: region, 401a: region, 401b: region, 402: region, 500: secondary battery, 501: positive electrode current collector, 502: positive electrode active material layer, 503: positive electrode, 504: negative electrode current collector, 505: negative electrode active material layer, 506: negative electrode, 507: separator, 507a: region, 507b: region, 508: electrolyte, 509: exterior body, 509a: exterior body, 509b: exterior body, 510: positive electrode lead electrode, 511: negative electrode lead electrode, 512: stack, 513: resin layer, 514: region, 515a: electrolyte, 515b: electrolyte, 515c: electrolyte, 516: inlet, 550: stack, 553: acetylene black, 554: graphene, 556: acetylene black, 557: graphene, 560: secondary battery, 561: positive electrode active material, 563: negative electrode active material, 570: manufacturing apparatus, 571: component introduction chamber, 572: transfer chamber, 573: processing chamber, 576: component extraction chamber, 580: transfer mechanism, 581: polymer film, 582: hole, 584: polymer film, 585: hole, 591: stage, 594: nozzle, 600: secondary battery, 701: commercial power source, 703: distribution board, 705: power storage controller, 706: indicator, 707: general load, 708: power storage load, 709: router, 710: service wire mounting portion, 711: measuring portion, 712: predicting portion, 713: planning portion, 790: control device, 791: power storage device, 796: underfloor space, 799: building, 903: mixture, 904: mixture, 904a: current collector, 904b: current collector, 905a: negative electrode active material layer, 905b: negative electrode active material layer, 911a: terminal, 911b: terminal, 913: secondary battery, 930: housing, 930a: housing, 930b: housing, 931: negative electrode, 931a: negative electrode active material layer, 932: positive electrode, 932a: positive electrode active material layer, 933: separator, 950: wound body, 950a: wound body, 951: terminal, 952: terminal, 970: secondary battery, 971: housing, 972: stack, 973a: positive electrode lead electrode, 973b: terminal, 973c: conductor, 974a: negative electrode lead electrode, 974b: terminal, 974c: conductor, 975a: positive electrode, 975b: positive electrode, 976: separator, 977a: negative electrode, 991a: graphite, 991b: graphite, 992a: frame, 992b: frame, 993a: frame, 993b: frame, 994a: frame, 994b: frame, 995a: frame, 995b: frame, 1301a: first battery, 1301b: first battery, 1302: battery controller, 1303: motor controller, 1304: motor, 1305: gear, 1306: DCDC circuit, 1307: electric power steering, 1308: heater, 1309: defogger, 1310: DCDC circuit, 1311: second battery, 1312: inverter, 1313: audio, 1314: power window, 1315: lamps, 1316: tire, 1317: rear motor, 1320: control circuit portion, 1321: control circuit portion, 1322: control circuit, 1324: switch portion, 1325: external terminal, 1326: external terminal, 1415: battery pack, 1421: wiring, 1422: wiring, 2001: motor vehicle, 2002: transporter, 2003: transport vehicle, 2004: aircraft, 2005: transport vehicle, 2100: electric bicycle, 2101: secondary battery, 2102: power storage device, 2103: display portion, 2104: control circuit, 2201: battery pack, 2202: battery pack, 2203: battery pack, 2204: battery pack, 2300: motor scooter, 2301: side mirror, 2302: power storage device, 2303: indicator light, 2304: under-seat storage unit, 2603: vehicle, 2604: charging device, 2610: solar panel, 2611: wiring, 2612: power storage device, 2800: personal computer, 2801: housing, 2802: housing, 2803: display portion, 2804: keyboard, 2805: pointing device, 2806: secondary battery, 2807: secondary battery, 7100: portable display device, 7101: housing, 7102: display portion, 7103: operation button, 7104: secondary battery, 7200: portable information terminal, 7201: housing, 7202: display portion, 7203: band, 7204: buckle, 7205: operation button, 7206: input/output terminal, 7207: icon, 7300: display device, 7304: display portion, 7400: mobile phone, 7401: housing, 7402: display portion, 7403: operation button, 7404: external connection port, 7405: speaker, 7406: microphone, 7407: secondary battery, 7500: electronic cigarette, 7501: atomizer, 7502: cartridge, 7504: secondary battery, 7600: tablet terminal, 7625: switch, 7627: switch, 7628: operation switch, 7629: fastener, 7630: housing, 7630a: housing, 7630b: housing, 7631: display portion, 7631a: display portion, 7631b: display portion, 7633: solar cell, 7634: charging and discharging control circuit, 7635: power storage unit, 7636: DCDC converter, 7637: converter, 7640: movable portion, 8000: display device, 8001: housing, 8002: display portion, 8003: speaker portion, 8004: secondary battery, 8030: SU, 8100: lighting device, 8101: housing, 8102: light source, 8103: secondary battery, 8104: ceiling, 8105: side wall, 8106: floor, 8107: window, 8200: indoor unit, 8201: housing, 8202: air outlet, 8203: secondary battery, 8204: outdoor unit, 8300: electric refrigerator-freezer, 8301: housing, 8302: refrigerator door, 8303: freezer door, 8304: secondary battery, 9000: glasses-type device, 9000a: frame, 9000b: display portion, 9001: headset-type device, 9001a: microphone part, 9001b: flexible pipe, 9001c: earphone portion, 9002: device, 9002a: housing, 9002b: secondary battery, 9003: device, 9003a: housing, 9003b: secondary battery, 9005: watch-type device, 9005a: display portion, 9005b: belt portion, 9006: belt-type device, 9006a: belt portion, 9006b: wireless power feeding and receiving portion, 9300: cleaning robot, 9301: housing, 9302: display portion, 9303: camera, 9304: brush, 9305: operation button, 9306: secondary battery, 9310: dust, 9400: robot, 9401: illuminance sensor, 9402: microphone, 9403: upper camera, 9404: speaker, 9405: display portion, 9406: lower camera, 9407: obstacle sensor, 9408: moving mechanism, 9409: secondary battery, 9500: flying object, 9501: propellers, 9502: camera, 9503: secondary battery, 9504: electronic component

1. A secondary battery comprising a positive electrode active material particle and an electrolyte,
wherein after constant current charging is performed in an environment at 60° C. with a current value of 0.5 C (note that 1 C=210 mA/g is satisfied) until a voltage reaches 4.5 V, a charging process of performing constant voltage charging until a current value reaches 0.2 C and a discharging process of performing constant current discharging with a current value of 0.5 C until a voltage reaches 3 V are alternately repeated 150 or more times, and then discharging is performed, lithium cobalt oxide that is a surface portion of the positive electrode active material particle has an O3 structure, and
wherein the electrolyte comprises an imidazolium cation.

2. The secondary battery according to claim 1, further comprising a negative electrode,
wherein the negative electrode comprises graphite.

3. The secondary battery according to claim 2,
wherein the negative electrode comprises a current collector and a negative electrode active material layer over the current collector, and
wherein a proportion of the graphite to total weight of the negative electrode active material layer is 50 weight % or more.

4. A secondary battery comprising a positive electrode active material and an electrolyte,
wherein the positive electrode active material is lithium cobalt oxide that has an O3 structure after charging and discharging are repeated, and
wherein the electrolyte comprises a compound represented by General Formula (G1). 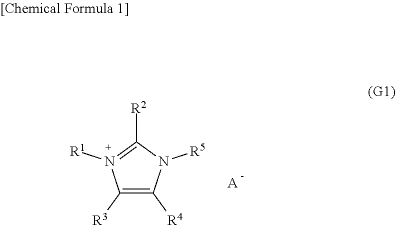
(In the formula, R¹ represents an alkyl group comprising 1 to 4 carbon atoms, R², R³, and R⁴ each independently represent a hydrogen atom or an alkyl group comprising 1 to 4 carbon atoms, and R⁵ represents an alkyl group or a main chain composed of two or more selected from C, O, Si, N, S, and P atoms. Moreover, A⁻ represents an amide-based anion represented by (CₙF₂ₙ₊₁SO₂)₂N⁻ (n is greater than or equal to 0 and less than or equal to 3).)

5. The secondary battery according to claim 4,
wherein in General Formula (G1), R¹ represents one selected from a methyl group, an ethyl group, and a propyl group,
wherein one of R², R³, and R⁴ represents a hydrogen atom or a methyl group and the other two represent hydrogen atoms,
wherein R⁵ represents an alkyl group or a main chain composed of two or more selected from C, O, Si, N, S, and P atoms, and
wherein A⁻ represents any of (FSO₂)₂N⁻ and (CF₃SO₂)₂N⁻ or a mixture thereof.

6. The secondary battery according to claim 4,
wherein in General Formula (G1), the sum of the number of carbon atoms of R¹, the number of carbon atoms of R⁵, and the number of oxygen atoms of R⁵ is 7 or less.

7. The secondary battery according to claim 4,
wherein in General Formula (G1), R¹ represents a methyl group, R² represents a hydrogen atom, and the sum of the numbers of carbon atoms and oxygen atoms of R⁵ is 6 or less.

8. The secondary battery according to claim 1,
wherein the electrolyte comprises one or more selected from a 1-butyl-3-propylimidazolium cation, a 1-ethyl-3-methylimidazolium cation, a 1-butyl-3-methylimidazolium cation, a 1-hexyl-3-methylimidazolium cation, and a 1-methyl-3-(2-propoxyethyl)imidazolium cation.

9. The secondary battery according to claim 1,
wherein the electrolyte comprises a 1-ethyl-3-methylimidazolium cation.

10. An electronic device comprising the secondary battery according to claim 1, a display portion, and a sensor.

11. A vehicle comprising the secondary battery according to claim 1, an electric motor, and a control device, and
wherein the control device is configured to supply electric power from the secondary battery to the electric motor.
